(19) United States
(12) Patent Application Publication
(10) Pub. No.: US 2024/0345498 A1
(43) Pub. Date: Oct. 17, 2024

(54) ELECTROPHOTOGRAPHIC PHOTORECEPTOR, ELECTROPHOTOGRAPHIC PHOTORECEPTOR CARTRIDGE, IMAGE FORMATION DEVICE, COATING LIQUID FOR FORMING ELECTROPHOTOGRAPHIC PHOTORECEPTOR PROTECTIVE LAYER, AND COMPOUND

(71) Applicant: MITSUBISHI CHEMICAL CORPORATION, Tokyo (JP)

(72) Inventors: Tsukasa HASEGAWA, Tokyo (JP); Manuel Emilio OTERO RAMIREZ, Tokyo (JP); Akira ANDO, Tokyo (JP); Hideki GOROUMARU, Tokyo (JP)

(73) Assignee: MITSUBISHI CHEMICAL CORPORATION, Tokyo (JP)

(21) Appl. No.: 18/754,100

(22) Filed: Jun. 25, 2024

Related U.S. Application Data
(63) Continuation of application No. PCT/JP2022/047851, filed on Dec. 26, 2022.

(30) Foreign Application Priority Data
Dec. 28, 2021 (JP) 2021-214074
Dec. 28, 2021 (JP) 2021-214075

(51) Int. Cl. G03G 5/147

(52) U.S. Cl. CPC G03G 5/14708; G03G 2215/00957; G03G 2215/00962; G03G 2221/183

(57) ABSTRACT

A novel compound having electron-transporting property which dissolves sufficiently in an organic solvent is provided.

### TECHNICAL FIELD

The present invention relates to an electrophotographic photoreceptor used for a copier, a printer or the like, an electrophotographic photoreceptor cartridge and an image formation device using the same and a coating liquid for forming an electrophotographic photoreceptor protective layer. The present invention also relates to a compound having electron-transporting property, such as a compound which is useful as an electron-transporting compound or the like that is a raw material of an electrophotographic photoreceptor used for a copier, a printer or the like.

### BACKGROUND ART

In a printer, a copier and the like, when light is applied to a charged organic photoconductor (OPC) drum, an electrostatic latent image is formed because the charge is eliminated from the part, and an image can be obtained because the toner adheres to the electrostatic latent image. Thus, in a device using electrophotographic technique, the photoreceptor is the key member.

Because there is considerable room for material selection for such a kind of organic photoconductor and because the characteristics of the photoreceptor are easily regulated, a “function-separated photoreceptor” in which the functions of generating and transporting the charge are assigned to different compounds has become the mainstream. For example, a single-layer electrophotographic photoreceptor (called a single-layer photoreceptor below) having a charge generation material (CGM) and a charge transport material (CTM) in the same layer and a laminate-type electrophotographic photoreceptor (called a laminate-type photoreceptor below) obtained by laminating a charge generation layer containing a charge generation material (CGM) and a charge transport layer containing a charge transport material (CTM) are known. Moreover, the charging methods of a photoreceptor include a negatively charging method for negatively charging the surface of a photoreceptor and a positively charging method for positively charging the surface of a photoreceptor.

Combinations of the layer configuration of a photoreceptor and the charging method which are currently used are “a negatively charged laminate-type photoreceptor” and “a positively charged single-layer photoreceptor”.

A general “negatively charged laminate-type photoreceptor” has a configuration obtained by providing an undercoat layer (UCL) composed of a resin or the like on a conductive base such as an aluminum tube, providing a charge generation layer (CGL) composed of a charge generation material (CGM), a resin and the like thereon and further providing a charge transport layer (CTL) composed of a hole transport material (HTM), a resin and the like thereon.

On the other hand, a general “positively charged single-layer photoreceptor” has a configuration obtained by providing an undercoat layer (UCL) composed of a resin or the like on a conductive base such as an aluminum tube and providing a single-layer photosensitive layer composed of a charge generation material (CGM), a hole transport material (HTM), an electron transport material (ETM), a resin and the like thereon (for example, see PTL 1).

In both photoreceptors, by charging the surface of the photoreceptor by a corona discharging method or a contact method and then exposing the photoreceptor to neutralize the charge on the surface, an electrostatic latent image is formed due to the potential difference from the surrounding surface. Then, a toner is brought into contact with the photoreceptor surface to form a toner image corresponding to the electrostatic latent image, and print is finished by transferring/heat-melt fixing the image on paper or the like.

As described above, a photosensitive layer is formed on a conductive support in the basic configuration of an electrophotographic photoreceptor, but a protective layer is sometimes provided on the photosensitive layer for the purpose of improving the abrasion resistance or the like.

As a technique for improving the mechanical strength or the abrasion resistance of the surface of a photoreceptor, a photoreceptor obtained by forming a layer containing a compound having a chain-polymerizable functional group as a binder resin on the outermost layer of the photoreceptor and applying energy such as heat, light and radiation to the layer to polymerize the compound to form a cured resin layer is disclosed (for example, see PTLs 1 and 2).

### CITATION LIST

### Patent Literature

PTL 1: US9,417,538B
PTL 2: WO2010/035683 (JP5263296B)

### SUMMARY OF INVENTION

### Technical Problem

As described above, a protective layer is provided to improve the abrasion resistance of the photoreceptor. In particular, a protective layer using a curable compound has particularly excellent mechanical strength.

Electron-transporting property is required for such a protective layer to improve the electrical properties of the photoreceptor. As means therefor, it is believed to be effective to add a compound having an electron-transporting structure to a protective layer using a curable compound. Specifically, a protective layer is generally formed by dissolving a curable composition containing a compound having electron-transporting property in an organic solvent to produce a coating liquid for forming a protective layer and coating the surface of the photoreceptor with the coating liquid for forming a protective layer.

It has been found, however, that compounds having an electron-transporting structure include those which do not dissolve in an organic solvent sufficiently when contained in a protective layer and those having insufficient electrical properties, in particular insufficient residual potential property and insufficient potential retention rate.

Thus, an object of the present invention is to provide a novel electrophotographic photoreceptor which has a photosensitive layer and a protective layer in this order on a conductive support and which can achieve electrical properties, in particular both residual potential property and potential retention rate, even when a compound having an electron-transporting structure is contained in the protective layer.

Another object of the present invention is to provide a novel compound having electron-transporting property which dissolves sufficiently in an organic solvent.

### Solution to Problem

As a result of examination by the present inventors, the electrophotographic photoreceptor, the electrophotographic photoreceptor cartridge, the image formation device, the coating liquid for forming an electrophotographic photoreceptor protective layer and the compound below are proposed to achieve the objects.

[1] An electrophotographic photoreceptor having at least a photosensitive layer and a protective layer in this order on a conductive support
in which the protective layer contains an electron-transporting compound represented by the following formula (1).

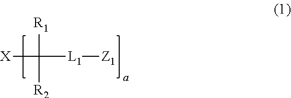

In the formula (1), X represents an electron-transporting skeleton. R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents. L₁ represents a divalent group. Z₁ represents an amide group (—NHCO—R′), an acrylamide group, a methacrylamide group, an acryloyl group or a methacryloyl group. Z₁ represents an amide group, an acrylamide group or a methacrylamide group when a is 1, and at least one thereof represents an amide group, an acrylamide group or a methacrylamide group when a is an integer of 2 or more. R′ represents a hydrogen atom, an alkyl group which may have one or more substituents, an aralkyl group which may have one or more substituents or an aromatic group which may have one or more substituents. a represents an integer of 1 or more. When a is an integer of 2 or more, R₁, R₂, L₁ and Z₁ in each of the repeating structures may be the same or different from each other.

[2] The electrophotographic photoreceptor described in [1] above in which the structure of X in the formula (1) in which the binding site has been replaced with a hydrogen atom is a structure selected from the group consisting of the formulae (A-1) to (A-13) shown in the following.

In the formulae (A-1) to (A-13) shown in the following, P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom. m1 to m10 each independently represent an integer of 0 or more. When m1 to m10 are each an integer of 2 or more, P₆ to P₁₅ in each of the repeating structures may be the same or different from each other. Q₁ to Q₂₄ each independently represent any of an oxygen atom, a sulfur atom, C(CN)₂, CR″CN, CA₂, C(COOR″)₂, CR″COOR″, NR″ and NCR″. A represents a halogen atom, and R″ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents. Ar₁ to Ar₁₉ each independently represent an aromatic group which may have one or more substituents or a heteroaromatic group which may have one or more substituents.

[3] The electrophotographic photoreceptor described in [1] or [2] above in which the structure of X in the formula (1) in which the binding site has been replaced with a hydrogen atom is a structure selected from the group consisting of the formulae (B-1) to (B-38) shown in the following.

In the formulae (B-1) to (B-38) shown in the following P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom. m1 to m10 each independently represent an integer of 0 or more. When m1 to m10 are each an integer of 2 or more, P₆ to P₁₅ in each of the repeating structures may be the same or different from each other.

[4] The electrophotographic photoreceptor described in any one of [1] to [3] above in which at least one of R₁ and R₂ in the formula (1) is an alkyl group which has two or more carbon atoms and which may have one or more substituents.

[5] The electrophotographic photoreceptor described in any one of [1] to [4] above in which the solubility of the electron-transporting compound represented by the formula (1) in methanol at 25° C. is three parts by mass or more.

[6] The electrophotographic photoreceptor described in any one of [1] to [5] above in which at least one or more of Z₁s in the formula (1) are an acrylamide group or a methacrylamide group.

[7] An electrophotographic photoreceptor cartridge having the electrophotographic photoreceptor described in any one of [1] to [6] above.

[8] An image formation device having the electrophotographic photoreceptor described in any one of [1] to [6] above.

[9] A coating liquid for forming an electrophotographic photoreceptor protective layer containing an electron-transporting compound represented by the formula (1) and a solvent.

[10] The coating liquid for forming an electrophotographic photoreceptor protective layer described in [9] above in which the solubility of the electron-transporting compound represented by the formula (1) in methanol at 25° C. is three parts by mass or more.

[11] The coating liquid for forming an electrophotographic photoreceptor protective layer described in [9] or [10] above which further contains a curable compound and in which the curable compound content is 10 parts by mass or less based on 100 parts by mass of the solvent.

[12] A compound represented by the formula (1).

[13] The compound described in [12] above in which the structure of X in the formula (1) in which the binding site has been replaced with a hydrogen atom is a structure selected from the group consisting of the formulae (A-1) to (A-13) shown in the following.

[14] The compound described in [12] or [13] above in which the structure of X in the formula (1) in which the binding site has been replaced with a hydrogen atom is a structure selected from the group consisting of the formulae (B-1) to (B-38) shown in the following.

[15] The compound described in any one of [12] to [14] above in which at least one or more of Z₁s in the formula (1) are an acrylamide group or a methacrylamide group.

### Advantageous Effects of Invention

The electrophotographic photoreceptor proposed in the present invention contains a certain compound having an electron-transporting structure and an amide bond structure in the protective layer and can improve the electron-transporting property in the protective layer and achieve electrical properties, in particular both residual potential property and potential retention rate. Moreover, the novel compound proposed in the present invention has an electron-transporting structure and an amide bond structure and thus has electron-transporting property and dissolves sufficiently in an organic solvent.

### BRIEF DESCRIPTION OF DRAWINGS

FIG. 1 is a figure schematically illustrating an example configuration of an image formation device which can be configured using the electrophotographic photoreceptor according to an example of the present invention.

FIG. 2 is a graph showing a general relation between the indentation depth of the indenter and the load curve in measurement of the Martens hardness and the elastic deformation ratio of a photoreceptor.

### DESCRIPTION OF EMBODIMENTS

Embodiments for carrying out the present invention (embodiments of the invention below) will be explained in detail below. Here, the present invention is not limited to the embodiments below and can be carried out with various modifications in the scope of the gist thereof.

### <<Compound of Present Invention>>

The compound according to an example of the embodiment of the present invention (referred to as “the compound of the present invention”) is preferably a compound represented by the following formula (1).

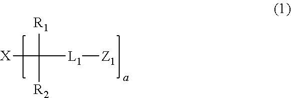

### [Electron-Transporting Skeleton: X]

In the formula (1), X is a structure having electron-transporting property, namely an electron-transporting skeleton. As the electron-transporting skeleton, a known electron-transporting skeleton can be appropriately used.

The electron-transporting skeleton can be an anthraquinone skeleton, a dinaphthoquinone skeleton, a benzene diimide skeleton, a naphthalene diimide skeleton, a perylene diimide skeleton, an isoindigo skeleton, a diketopyrrolopyrrole skeleton, a thiadiazole skeleton, a pyrazine skeleton or the like. Of these, in view of the electron-transporting property and the solubility in an organic solvent, a dinaphthoquinone skeleton, a benzene diimide skeleton, a naphthalene diimide skeleton or a perylene diimide skeleton is preferable, and a benzene diimide skeleton, a naphthalene diimide skeleton or a perylene diimide skeleton is more preferable. A benzene diimide skeleton or a naphthalene diimide skeleton is further preferable.

The structure of X in the formula (1) in which the binding site has been replaced with a hydrogen atom is preferably a structure selected from the group consisting of the following formulae (A-1) to (A-13).

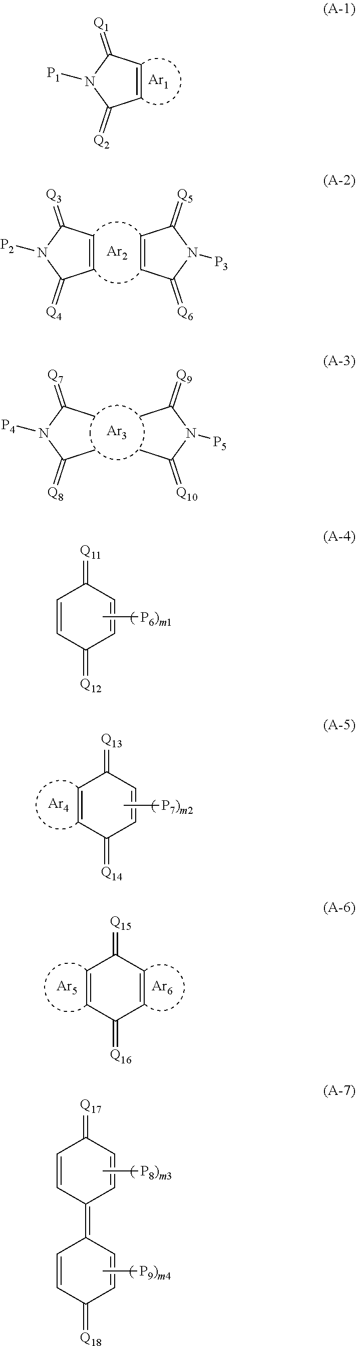

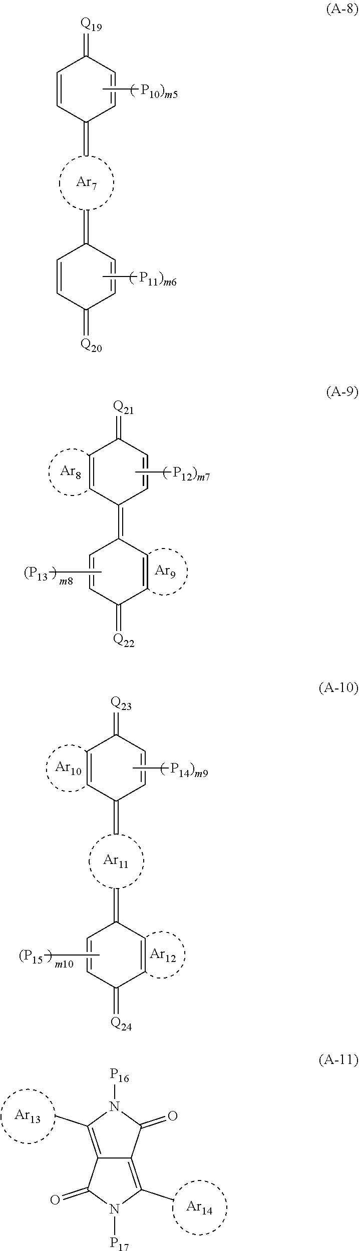

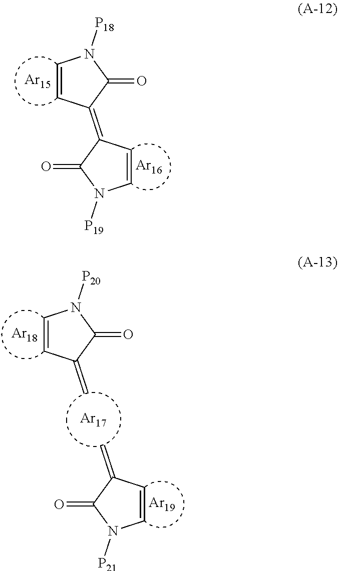

In the formulae (A-1) to (A-13), P₁ to P₂₁ are preferably each independently a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom.

Of these, in view of the solubility and the curability, P₁ to P₂₁ are more preferably a hydrogen atom or an alkyl group which may have one or more substituents, further preferably a hydrogen atom.

Here, “which may have one or more substituents” in the present invention means that the group can have one or more substituents and has the meanings including both having one or more substituents and having no substituent.

In the compound of the present invention, the substituents of the alkyl group which may have one or more substituents and the like include an alkyl group, an alkoxy group, an aryloxy group, a heteroaryloxy group, an alkoxycarbonyl group, a dialkylamino group, a diarylamino group, an arylalkylamino group, an acyl group, a haloalkyl group, an alkylthio group, an arylthio group, a silyl group, a siloxy group, an aromatic hydrocarbon group, an aromatic heterocyclic group and the like. In view of the solubility, the substituents are preferably alkyl groups when the groups have substituents, and the groups more preferably have no substituent.

In the formulae (A-1) to (A-13), m1 to m10 are each independently an integer of 0 or more. Of these, in view of the solubility and the curability, m1 to m10 are preferably each independently an integer of 1 or more.

Here, when m1 to m10 are each an integer of 2 or more, P₆ to P₁₅ in each of the repeating structures may be the same or different from each other.

In the formulae (A-1) to (A-13), Q₁ to Q₂₄ are preferably each independently any of an oxygen atom, a sulfur atom, C(CN)₂, CR″CN, CA₂, C(COOR″)₂, CR″COOR″, NR″ and NCR″. Of these, in view of the electron-transporting property, an oxygen atom, C(CN)₂ or CR″CN is preferable, and an oxygen atom or C(CN)₂ is more preferable.

Here, A represents a halogen atom, and R″ is preferably a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents. Of these, in view of the solubility, R″ is preferably an alkyl group, an alkoxy group or an aromatic hydrocarbon group, more preferably an alkyl group.

In the formulae (A-1) to (A-13), Ar₁ to Ar₁₉ are preferably each independently an aromatic group which may have one or more substituents or a heteroaromatic group which may have one or more substituents. Of these, in view of the solubility, Ar₁ to Ar₁₉ are more preferably an aromatic group which may have one or more substituents.

Of the formulae (A-1) to (A-13), in view of the electron-transporting property, (A-1), (A-2), (A-3), (A-6) or (A-9) is preferable, and (A-2), (A-3) or (A-9) is more preferable. (A-2) or (A-3) is further preferable.

Of these, the structure of X in the formula (1) in which the binding site has been replaced with a hydrogen atom is preferably a structure selected from the group consisting of the following formulae (B-1) to (B-38).

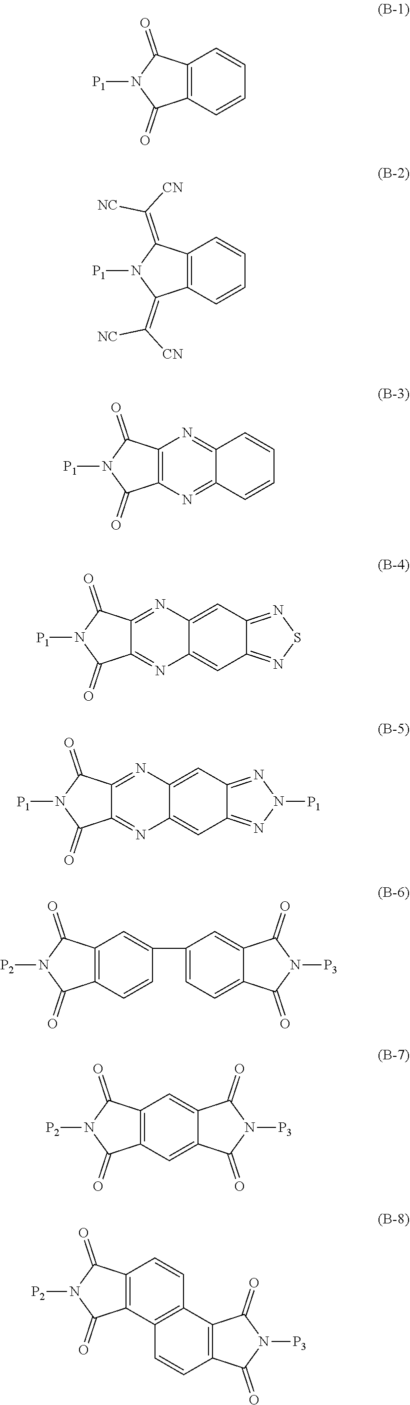

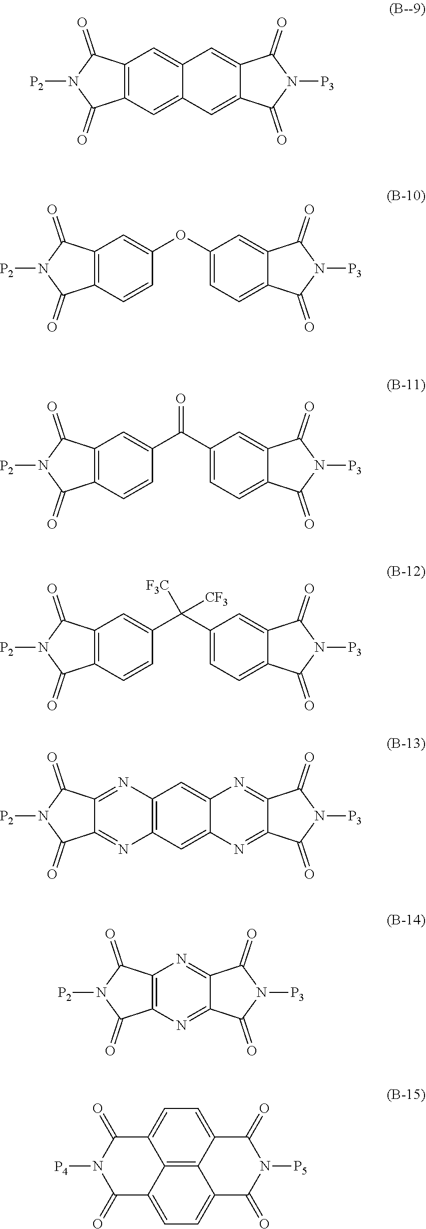

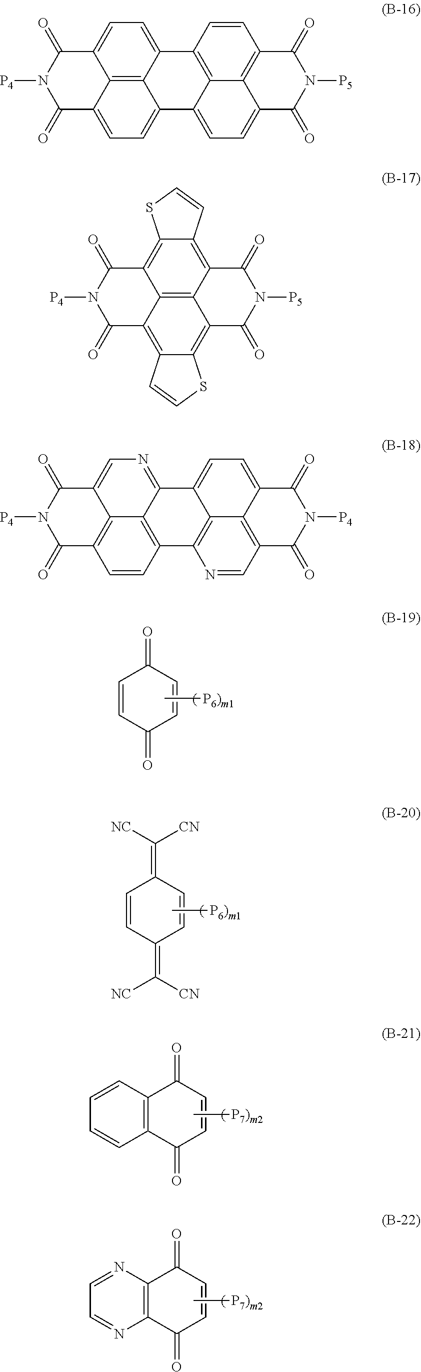

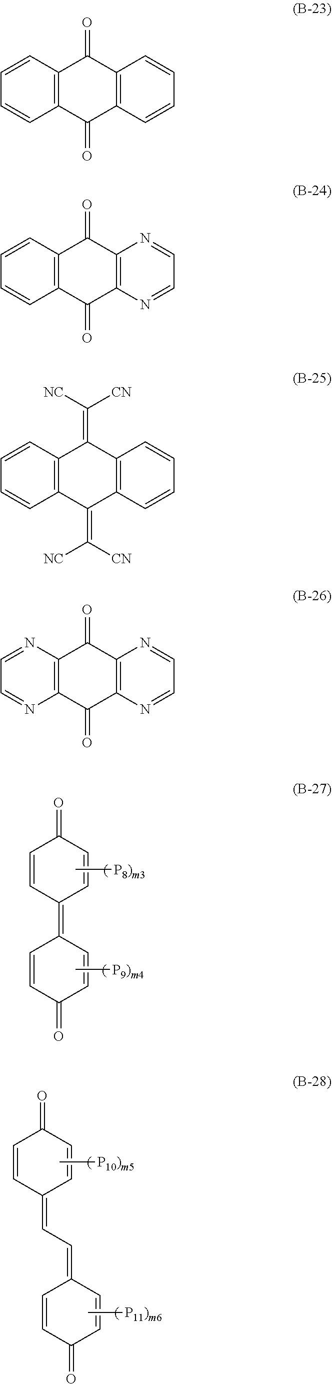

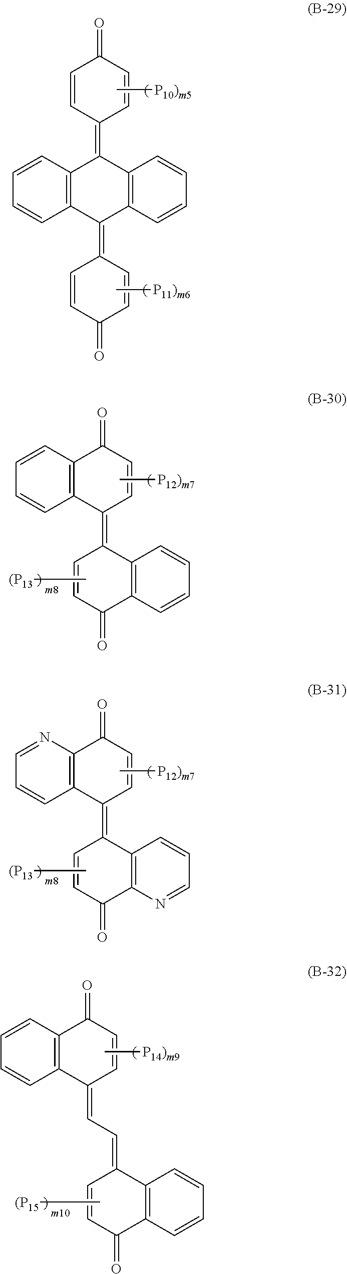

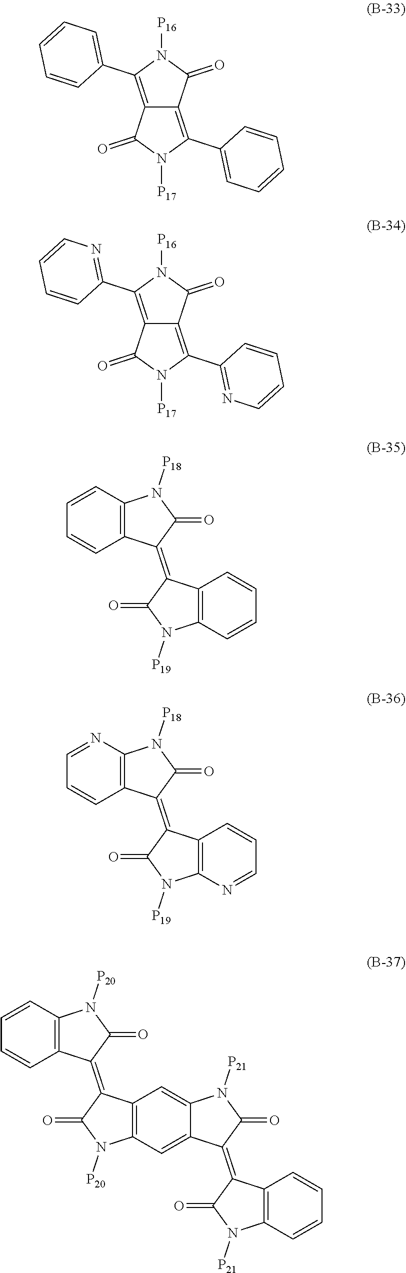

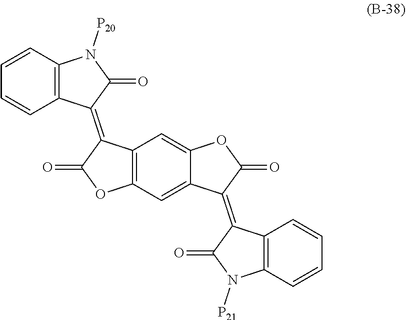

In the formulae (B-1) to (B-38), P₁ to P₂₁ are preferably each independently a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom.

Of these, in view of the solubility and the curability, P₁ to P₂₁ are more preferably a hydrogen atom or an alkyl group which may have one or more substituents, further preferably a hydrogen atom.

In the formulae (B-1) to (B-38), m1 to m10 are each independently an integer of 0 or more. Of these, in view of the solubility and the curability, m1 to m10 are preferably each independently an integer of 1 or more.

Here, when m1 to m10 are each an integer of 2 or more, P₆ to P₁₅ in each of the repeating structures may be the same or different from each other.

Of the formulae (B-1) to (B-38), in view of the solubility and the electron-transporting property, (B-1), (B-2), (B-7), (B-12), (B-14), (B-15), (B-16), (B-24) or (B-30) is preferable, and (B-7), (B-12), (B-14), (B-15), (B-16) or (B-30) is more preferable. (B-7), (B-12) or (B-15) is further preferable, and (B-7) or (B-12) is particularly preferable. (B-7) is most preferable.

### [Z₁]

In the formula (1), Z₁ represents an amide group (—NHCO—R′), an acrylamide group, a methacrylamide group, an acryloyl group or a methacryloyl group. When a is 1, Z₁ is preferably an amide group, an acrylamide group or a methacrylamide group. When a is an integer of 2 or more, at least one Z₁ is preferably an amide group, an acrylamide group or a methacrylamide group.

Here, R′ in the amide group (—NHCO—R′) represents a hydrogen atom, an alkyl group which may have one or more substituents, an aralkyl group which may have one or more substituents or an aromatic group which may have one or more substituents. Of these, in view of the solubility, R′ is preferably an alkyl group which may have one or more substituents.

As described above, Z₁ is preferably an amide group, an acrylamide group or a methacrylamide group when a is 1. When a is an integer of 2 or more, at least one Z₁ is preferably an amide group, an acrylamide group or a methacrylamide group. That is, the compound of the present invention preferably has at least any one of an amide group, an acrylamide group and a methacrylamide group.

The compound of the present invention has an electron-transporting structure and further has an amide bond, particularly a moiety of NH. Thus, when the compound is contained in a coating liquid for forming a protective layer of an electrophotographic photoreceptor, the affinity for the organic solvent used for the coating liquid, in particular an alcoholic organic solvent, becomes excellent, and the solubility becomes excellent. Therefore, the coatability of the coating liquid becomes excellent, and a uniform protective layer without unevenness can be formed. It is believed that, as a result, the electron-transporting property in the protective layer becomes excellent and that the electrical properties of the photoreceptor, in particular the residual potential property and the potential retention rate, become excellent.

In the formula (1), Z₁ is more preferably an amide group (—NHCO—R′), an acrylamide group, a methacrylamide group, an acryloyl group or a methacryloyl group, further preferably an acrylamide group or a methacrylamide group to improve the mechanical strength of the protective layer. In other words, in the formula (1), at least one or more of Z₁s are further preferably an acrylamide group or a methacrylamide group.

In this manner, when the amide bond is contained in the structure of the compound as an acrylamide group or a methacrylamide group, the amide bond can also play the role of a chain-polymerizable functional group and can be cross-linked with the curable compound in the protective layer. Thus, the mechanical strength of the protective layer, such as the hardness and the elastic deformation ratio, becomes further excellent.

[R₁ and R₂]

In the formula (1), R₁ and R₂ are preferably each independently a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents.

Of these, in view of the solubility in an organic solvent, a hydrogen atom or an alkyl group which may have one or more substituents is further preferable.

In the formula (1), at least one of R₁ and R₂ is particularly preferably an alkyl group which has two or more carbon atoms and which may have one or more substituents because a further excellent effect in terms of the solubility and the dark decay can be obtained.

### [L₁]

In the formula (1), L₁ is a divalent group. Examples thereof include an alkylene group, a divalent group having a ketone group, a divalent group having an ether bond, a divalent group having an ester bond, a group in which these groups are linked and the like. L₁, however, is not limited to these examples. Of these, in view of the solubility in an organic solvent, structures represented by the following formulae (L-1) to (L-5) are preferable, and of these, the formula (L-3), the formula (L-4) or the formula (L-5) is more preferable.

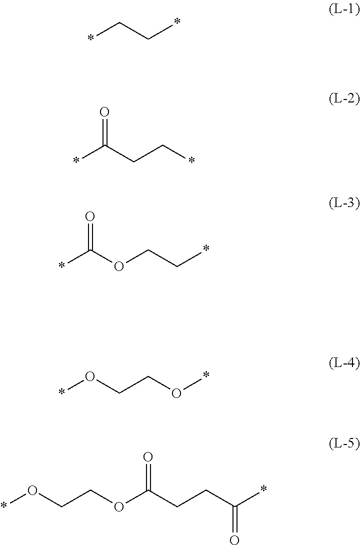

In the formulae (L-1) to (L-5), * represents the binding site to the formula (1).

In the formula (1), L₁ is a linker linking the electron-transporting skeleton and Z₁. It is believed that Z₁ is not easily affected by the interaction with the electron-transporting skeleton when Z₁ and the electron-transporting skeleton are apart and that, especially when Z₁ contains an amide bond, the effect of the amide bond, namely the effect of improving the affinity for the solvent, becomes strong. From this point, the number of the atoms of the main chain of L₁ is believed to be preferably four or more.

[a]

In the formula (1), the part other than X, which is an electron-transporting skeleton, may be repeating structures, and a in the formula (1) shows the number of the repeating structures.

That is, in the formula (1), a is an integer of 1 or more, in particular, preferably 2 or more in view of the solubility and the curability.

Here, when a is an integer of 2 or more, R₁, R₂, L₁ and Z₁ in each of the repeating structures in the formula (1) may be the same or different from each other.

### <Specific Examples of Compound of Present Invention>

Specific examples of the compound of the present invention are shown below. The compound, however, is not limited to the examples.

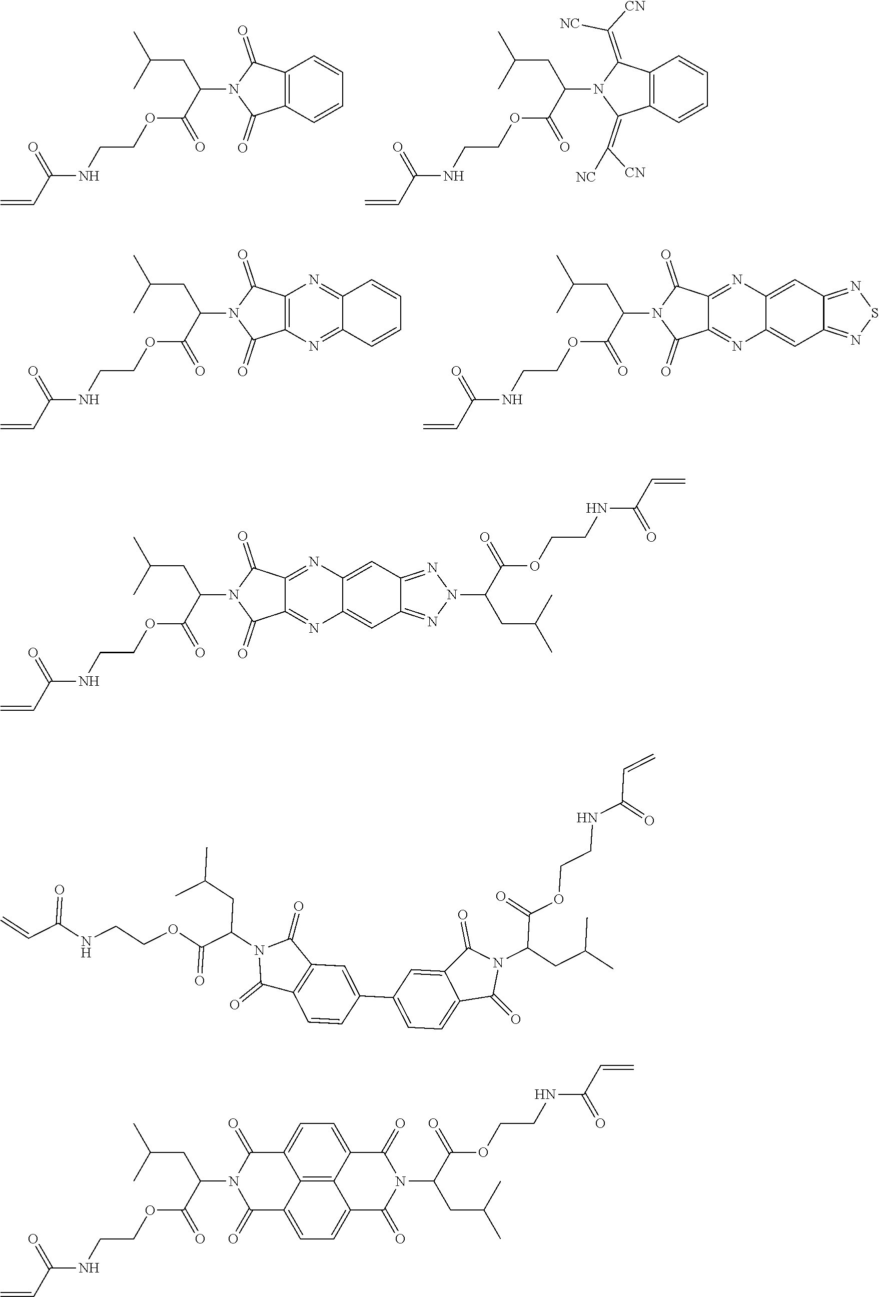

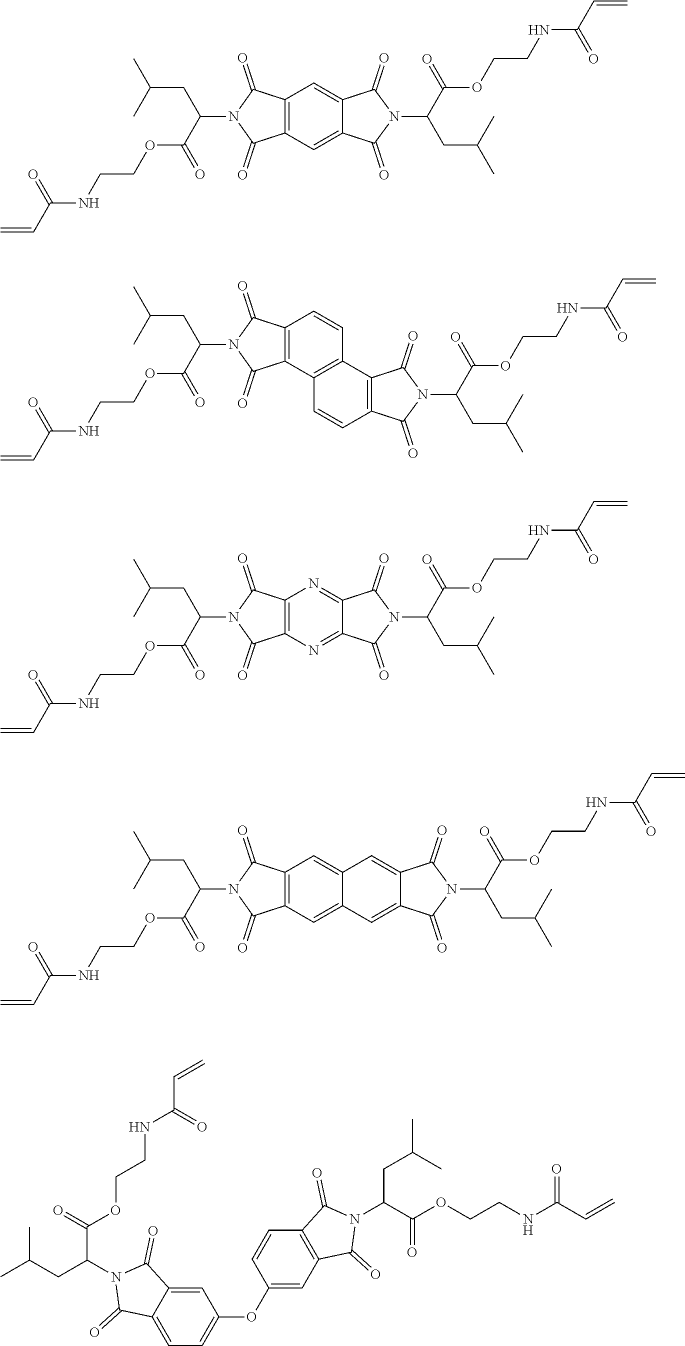

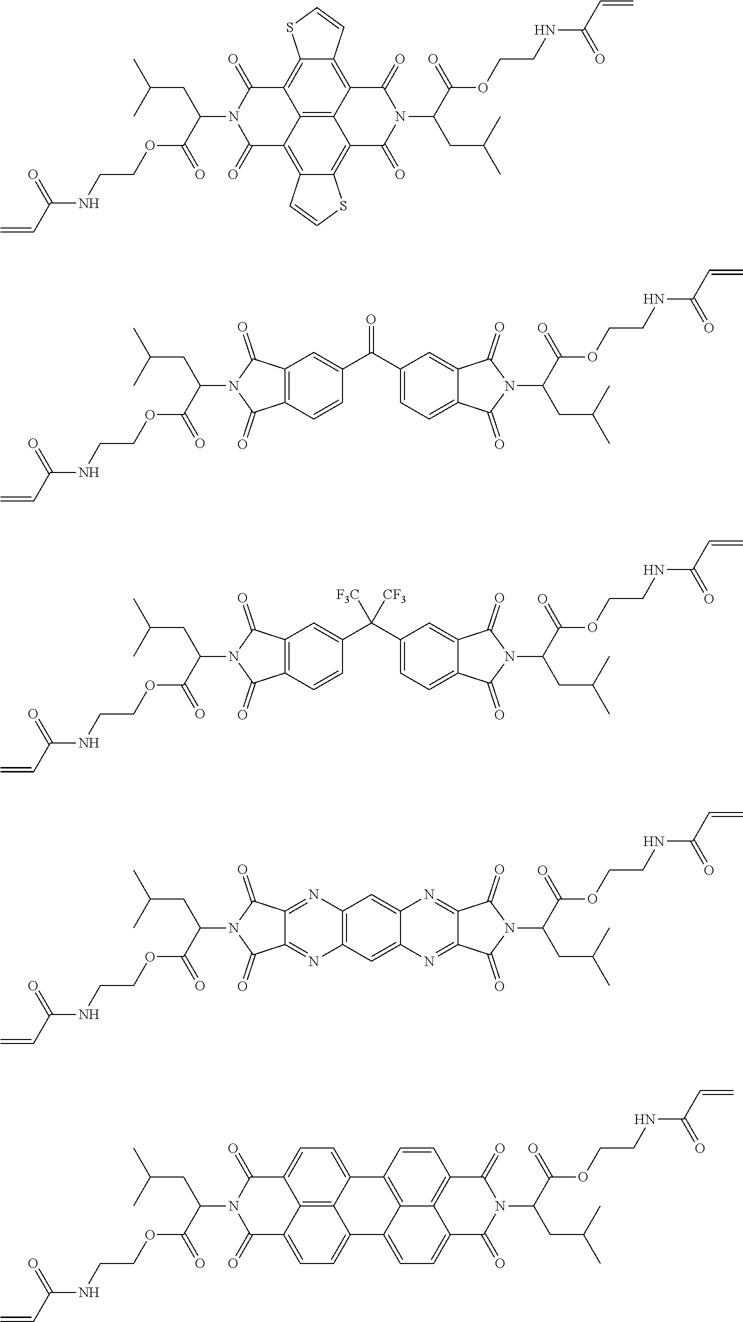

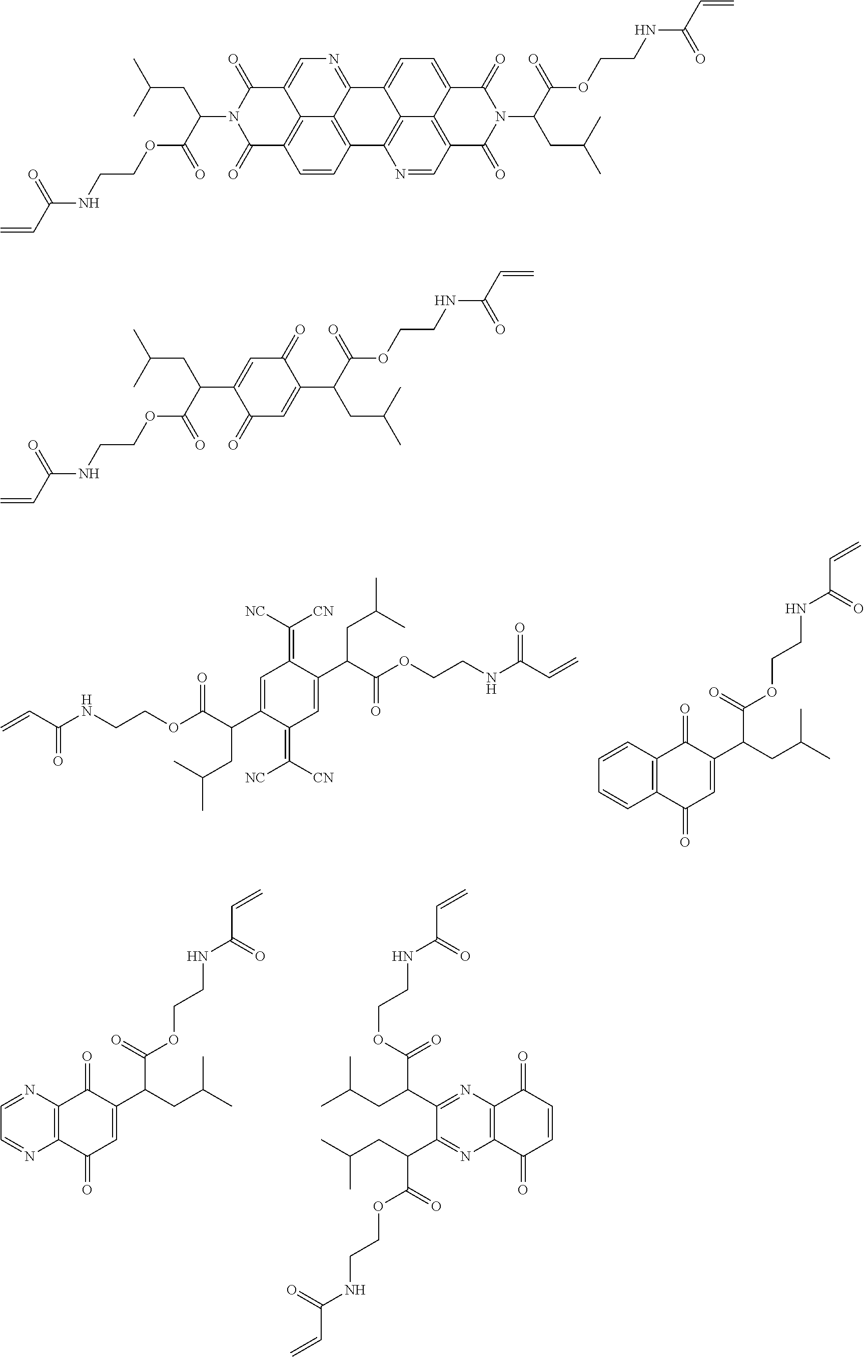

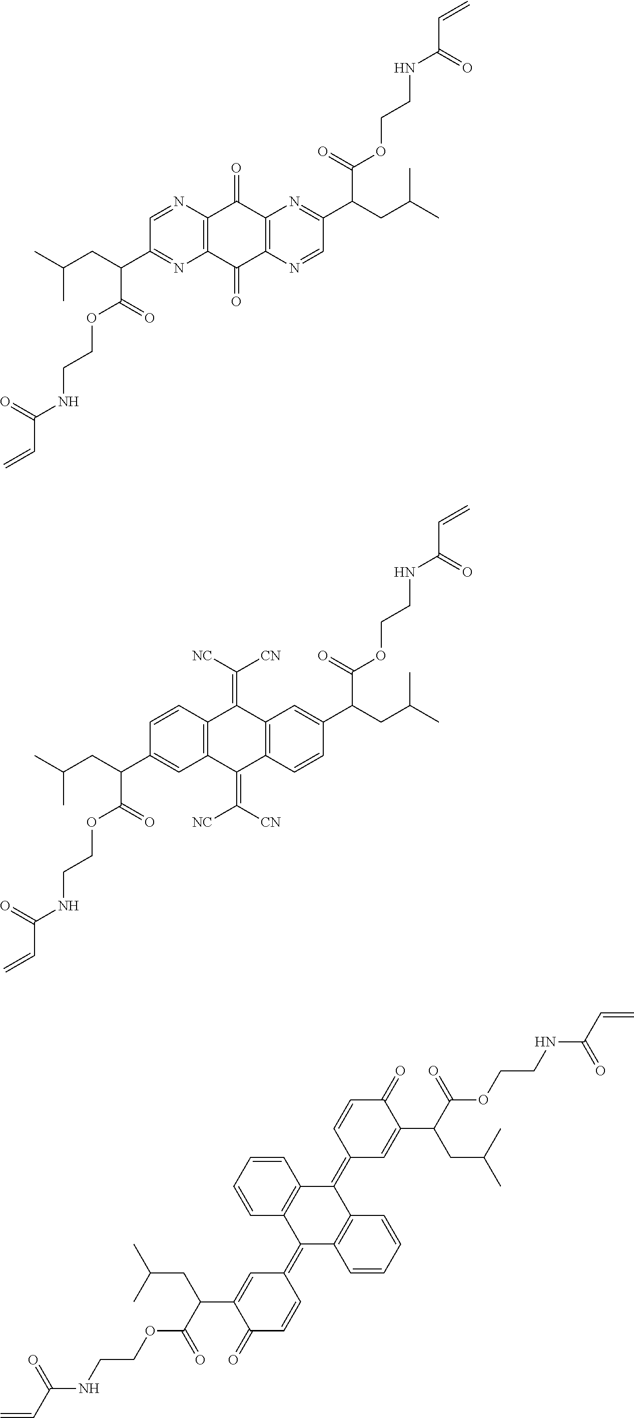

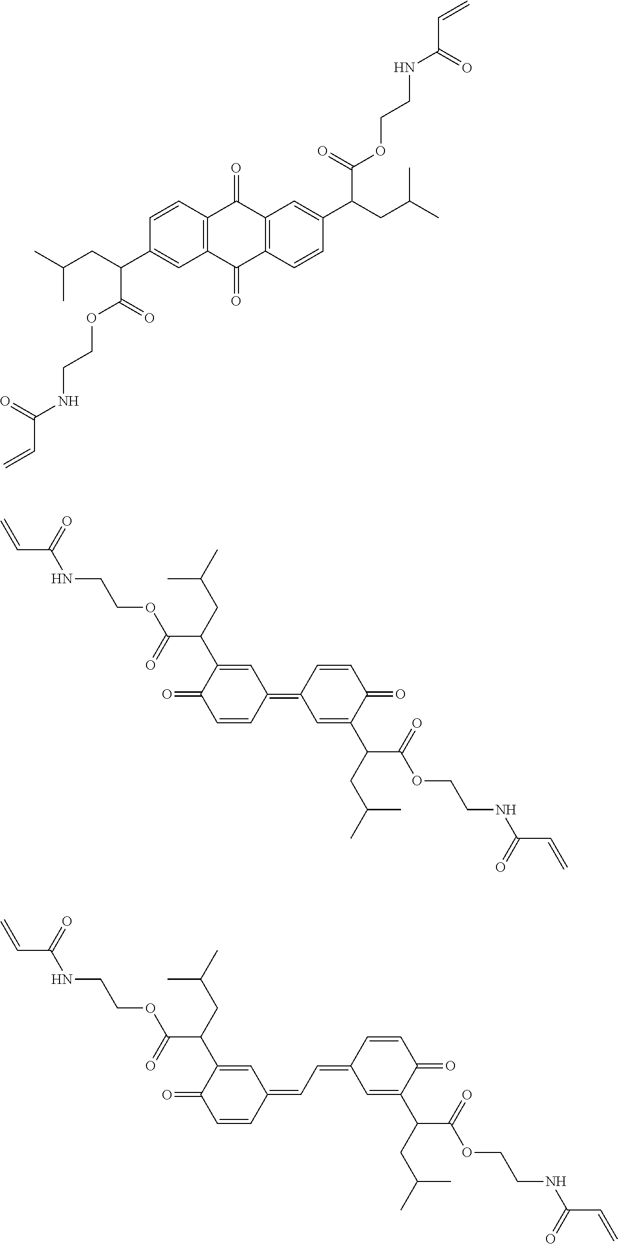

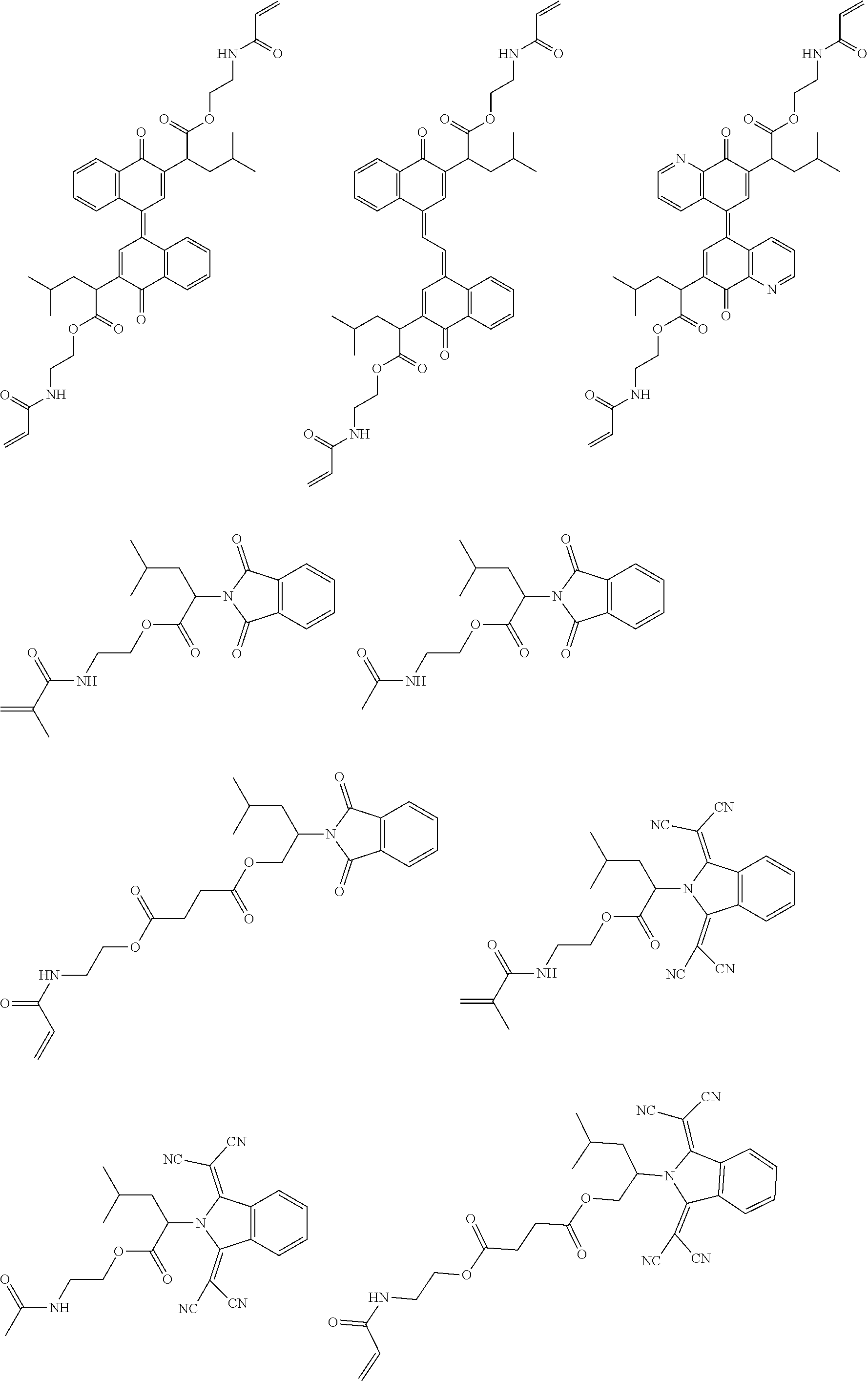

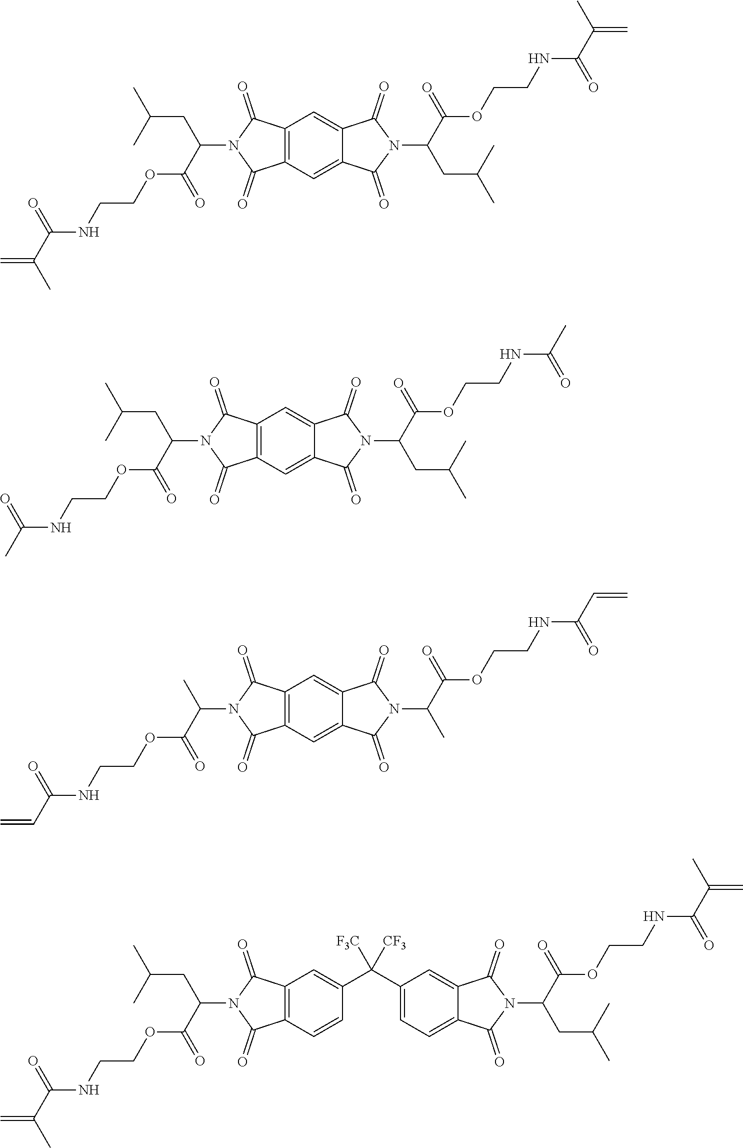

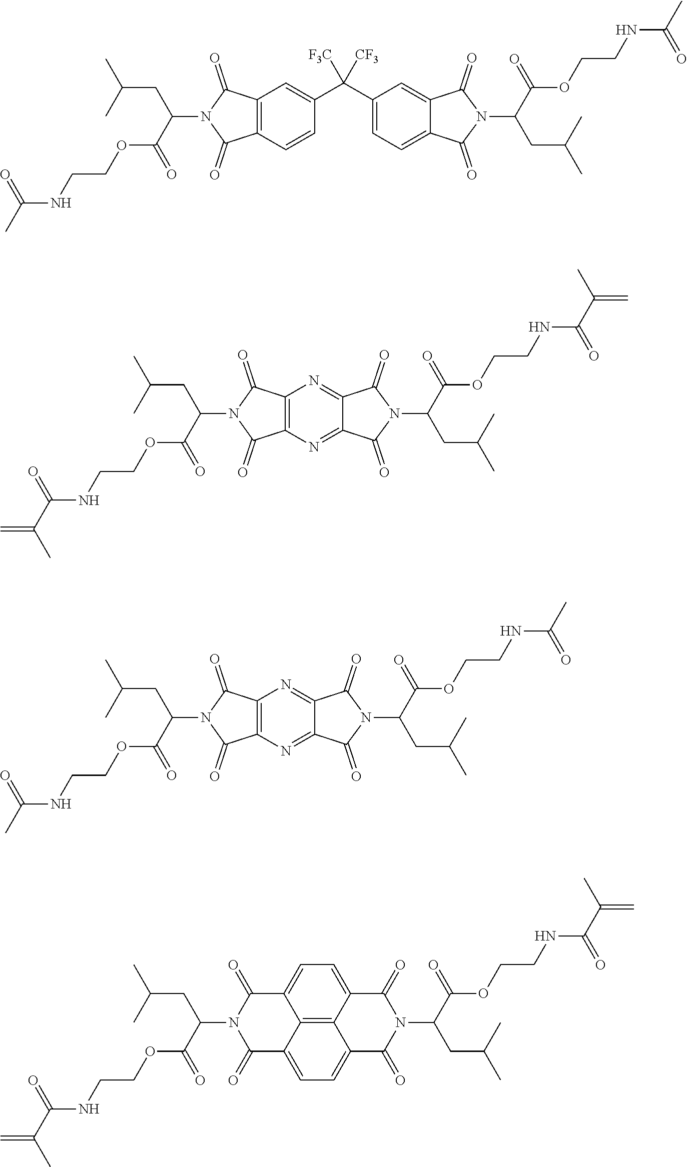

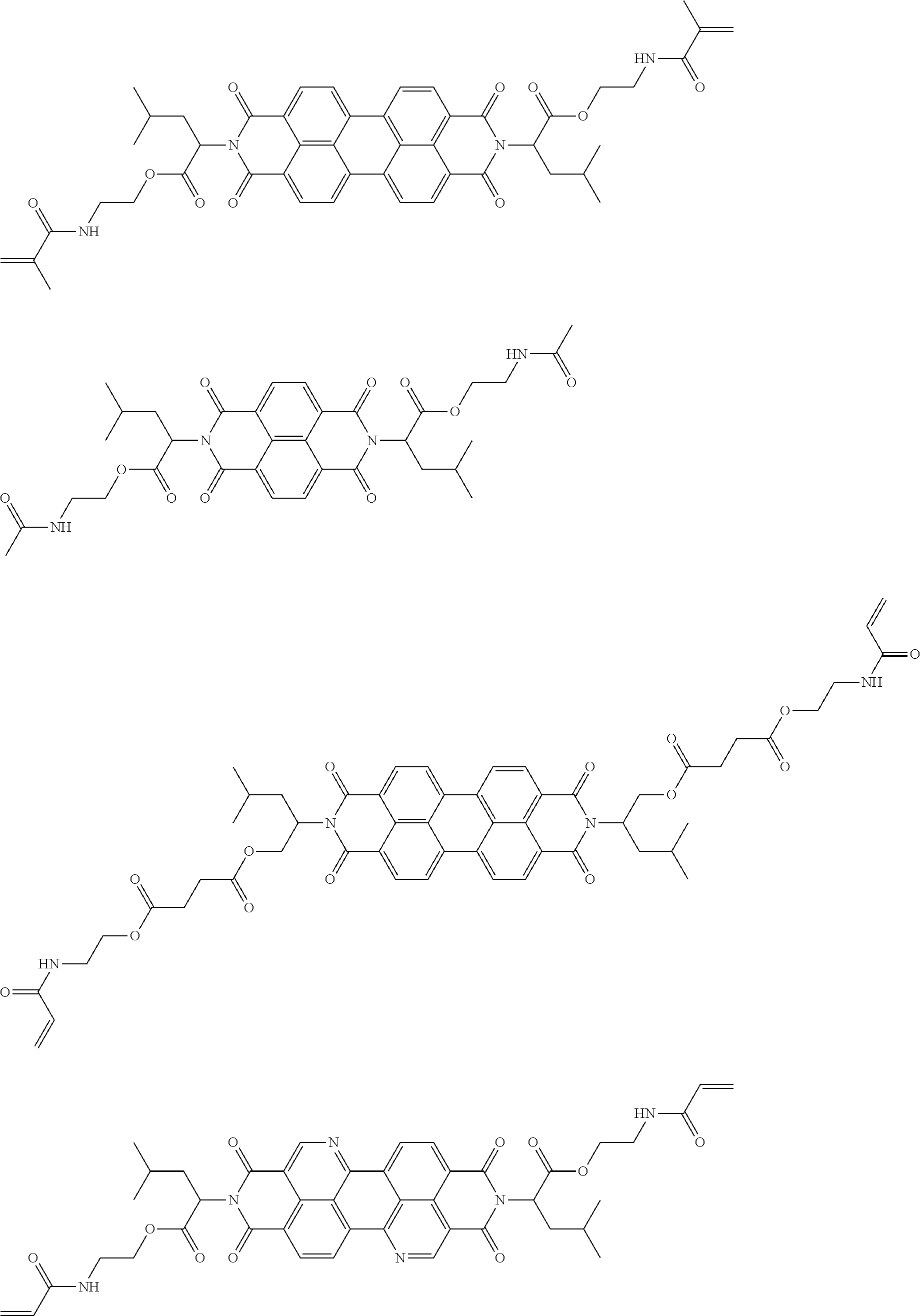

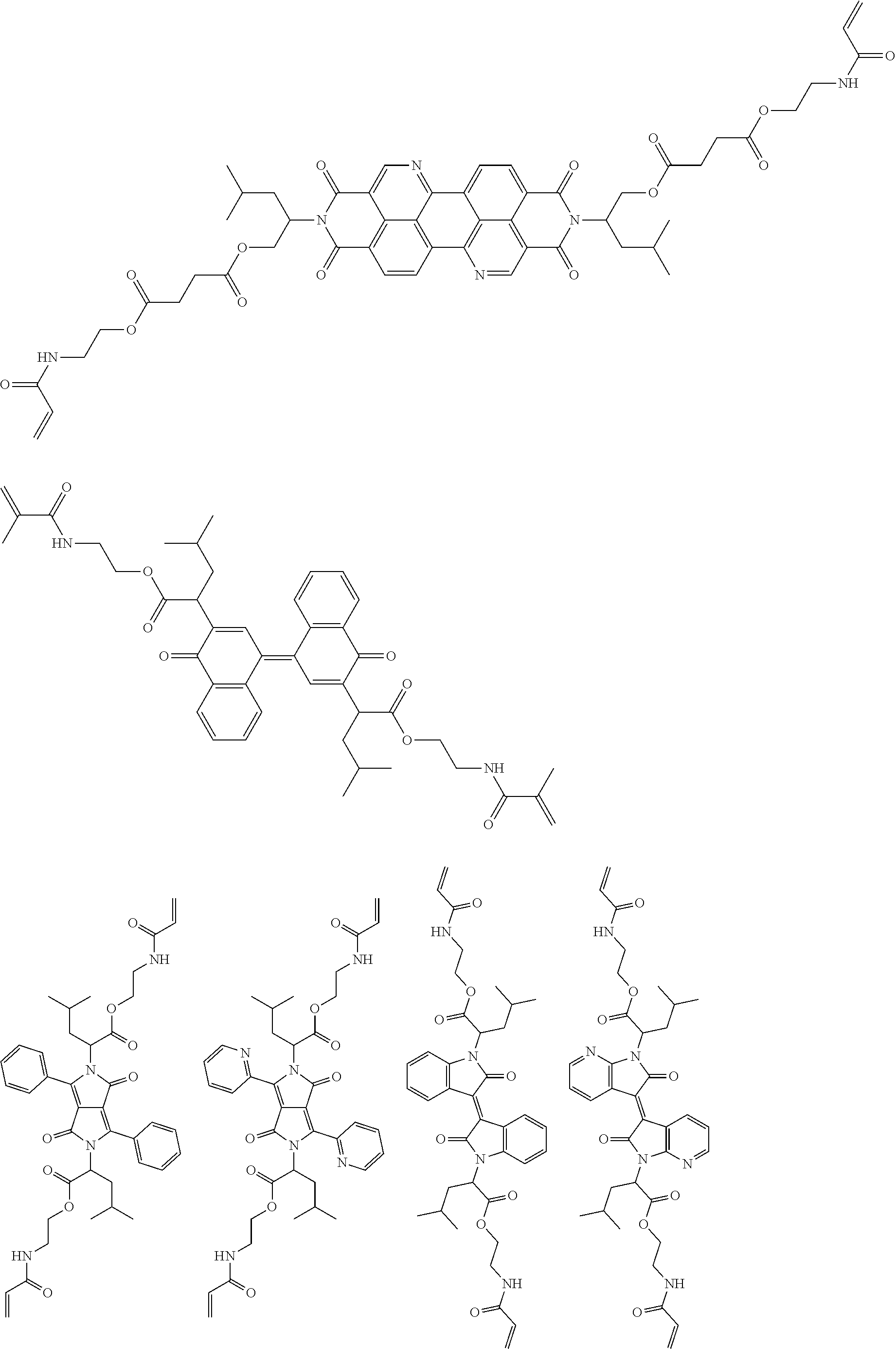

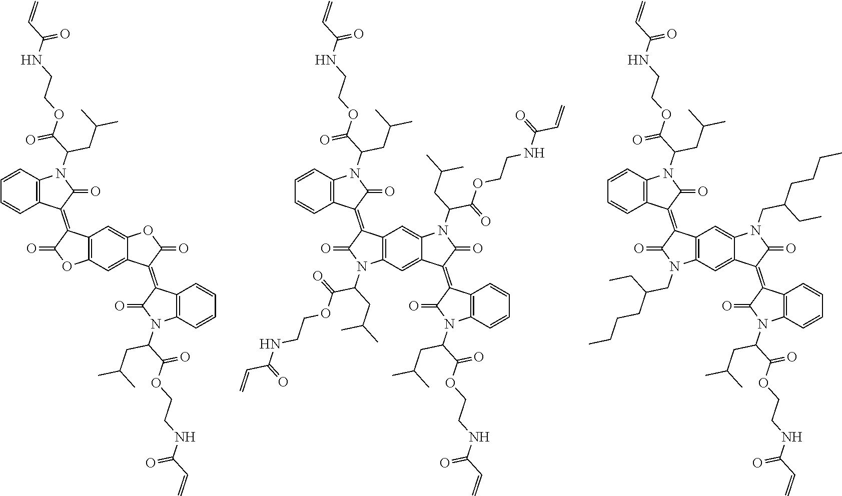 [SMILES: C=CC(=O)NCCOC(=O)C(CC(C)C)N1C(=O)/C(=C2/C(=O)Oc3cc4c(cc32)OC(=O)/C4=C2/C(=O)N(C(CC(C)C)C(=O)OCCNC(=O)C=C)c3ccccc32)c2ccccc21 ; C=CC(=O)NCCOC(=O)C(CC(C)C)N1C(=O)/C(=C2/C(=O)N(C(CC(C)C)C(=O)OCCNC(=O)C=C)c3cc4c(cc32)N(C(CC(C)C)C(=O)OCCNC(=O)C=C)C(=O)/C4=C2/C(=O)N(C(CC(C)C)C(=O)OCCNC(=O)C=C)c3ccccc32)c2ccccc21 ; C=CC(=O)NCCOC(=O)C(CC(C)C)N1C(=O)/C(=C2/C(=O)N(CC(CC)CCCC)c3cc4c(cc32)N(CC(CC)CCCC)C(=O)/C4=C2/C(=O)N(C(CC(C)C)C(=O)OCCNC(=O)C=C)c3ccccc32)c2ccccc21]

### <Solubility>

The solubility of the compound of the present invention in methanol at 25° C. is preferably three parts by mass or more.

In the present invention, “the solubility in methanol at 25° C.” indicates the maximum amount (parts by mass) of the compound which can be dissolved in 100 parts by mass of methanol under the condition at 25° C.

The solubility of the compound of the present invention in methanol at 25° C. is preferably three parts by mass or more, more preferably five parts by mass or more, further preferably six parts by mass or more, particularly preferably eight parts by mass or more.

### <<Electrophotographic Photoreceptor of Present Invention>>

The electrophotographic photoreceptor according to an example of the embodiment of the present invention (also referred to as “the electrophotographic photoreceptor of the present invention”) is an electrophotographic photoreceptor having at least a photosensitive layer and a protective layer (also referred to as “the protective layer of the present invention”) in this order on a conductive support.

The electrophotographic photoreceptor of the present invention can optionally have a layer other than the photosensitive layer and the protective layer.

Moreover, the charging method of the electrophotographic photoreceptor of the present invention may be either a negatively charging method for negatively charging the surface of the photoreceptor or a positively charging method for positively charging the surface of the photoreceptor. Of these, considering that electron-transporting property is required for the protective layer, the positively charging method is preferable because the effect of the present invention is believed to be exhibited more by the positively charging method.

In the electrophotographic photoreceptor of the present invention, the side opposite to the conductive support is the upper side or the surface side, and the conductive support side is the lower side or the back surface side.

### <Protective Layer of Present Invention>

The protective layer of the present invention is preferably a layer containing an electron-transporting compound, preferably a layer further containing a cured material obtained by curing a curable compound.

Here, the “electron-transporting compound” means a compound having electron-transporting property, in other words, a compound having an electron-transporting skeleton.

When the compound of the present invention has a polymerizable functional group (an acrylamide group, a methacrylamide group, an acryloyl group or a methacryloyl group) in the formula (1), the compound may be in the form of a polymer in the protective layer after curing. For example, the compound of the present invention may be a polymer in which the molecules of the compound are polymerized or a polymer polymerized with a curable compound when the protective layer contains a curable compound.

The protective layer of the present invention can be formed, for example, from a composition containing an electron-transporting compound and containing, according to the need, a curable compound, a polymerization initiator, inorganic particles and another material. However, the protective layer of the present invention is not limited to the layer formed from such a composition.

### (Electron-Transporting Compound)

The electron-transporting compound used for the protective layer of the present invention is preferably the compound of the present invention described above, namely the compound represented by the formula (1).

In view of the electron-transporting property, the electron-transporting compound content of the protective layer of the present invention, based on the total mass of 100 parts by mass of the protective layer of the present invention, is preferably 40 parts by mass or more, more preferably 60 parts by mass or more, further preferably 80 parts by mass or more.

### (Curable Compound)

The curable compound is a compound having a chain-polymerizable functional group. In particular, a monomer, an oligomer or a polymer having a radically polymerizable functional group is preferable. Of these, a curable compound having cross-linking property, in particular, a photocurable compound, is preferable. An example thereof is a curable compound having two or more radically polymerizable functional groups. A compound having one radically polymerizable functional group can also be used in combination.

The radically polymerizable functional group can be one of an acryloyl group (including an acryloyloxy group) and a methacryloyl group (including a methacryloyloxy group) or both groups.

Examples of the compounds which are preferable as the curable compound having a radically polymerizable functional group are shown below.

Examples of the monomer having an acryloyl group or a methacryloyl group include trimethylolpropane triacrylate (TMPTA), trimethylolpropane trimethacrylate, pentaerythritol triacrylate, pentaerythritol tetraacrylate, glycerol triacrylate, tris(acryloxyethyl)isocyanurate, dipentaerythritol hexaacrylate, dimethylolpropane tetraacrylate, pentaerythritol ethoxytetraacrylate, EO-modified phosphoric acid triacrylate, 2,2,5,5-tetrahydroxymethylcyclopentanone tetraacrylate, 2-hydroxy-3-acryloyloxy propylmethacrylate, polyethylene glycol diacrylate, polypropylene glycol diacrylate, polytetramethyleneglycol diacrylate, EO-modified bisphenol A diacrylate, PO-modified bisphenol A diacrylate, 9,9-bis[4-(2-acryloyloxyethoxy)phenyl]fluorene, tricyclodecane dimethanol diacrylate, decanediol diacrylate, hexanediol diacrylate, ethylene glycol dimethacrylate, polyethylene glycol dimethacrylate, EO-modified bisphenol A dimethacrylate, PO-modified bisphenol A dimethacrylate, tricyclodecane dimethanol dimethacrylate, decanediol dimethacrylate, hexanediol dimethacrylate and the like.

Moreover, examples of the oligomer and the polymer having an acryloyl group or a methacryloyl group include urethane acrylate, ester acrylate, acrylic acrylate, epoxy acrylate and the like. Of these, urethane acrylate and ester acrylate are preferable, and of these, ester acrylate is more preferable.

A kind of the compounds above can be used alone, or two or more kinds thereof can be used in combination.

In view of the electron-transporting property, the ratio (mass ratio) of the curable compound to the electron-transporting compound in the protective layer of the present invention is preferably 1.0 or less, more preferably 0.5 or less, further preferably 0.1 or less.

### (Polymerization Initiator)

The polymerization initiator can be a thermal polymerization initiator, a photopolymerization initiator or the like.

Examples of the thermal polymerization initiator include peroxide-based compounds such as 2,5-dimethylhexane-2,5-dihydroperoxide and azo-based compounds such as 2,2′-azobis(isobutyronitrile).

Photopolymerization initiators can be classified into a direct cleavage type and a hydrogen extraction type based on the difference in the radical generation mechanism.

A photopolymerization initiator of the direct cleavage type generates a radical because a part of the covalent bond in the molecule is cleaved when light energy is absorbed. On the other hand, a photopolymerization initiator of the hydrogen extraction type generates a radical because the molecule in the excited state after absorbing light energy extracts hydrogen from a hydrogen donor.

Examples of the photopolymerization initiator of the direct cleavage type include acetophenone-based or ketal-based compounds such as acetophenone, 2-benzoyl-2-propanol, 1-benzoylcyclohexanol, 2,2-diethoxyacetophenone, benzyldimethylketal and 2-methyl-4′-(methylthio)-2-morpholino propiophenone, benzoin ether-based compounds such as benzoin, benzoin methylether, benzoin ethylether, benzoin isobutylether, benzoin isopropylether and O-tosylbenzoin and acylphosphine oxide-based compounds such as diphenyl(2,4,6-trimethylbenzoyl)phosphine oxide, phenylbis(2,4,6-trimethylbenzoyl)phosphine oxide and lithium phenyl(2,4,6-trimethylbenzoyl)phosphonate.

Examples of the photopolymerization initiator of the hydrogen extraction type include benzophenone-based compounds such as benzophenone, 4-benzoylbenzoic acid, 2-benzoylbenzoic acid, methyl 2-benzoylbenzoate, methyl benzoylformate, benzyl, p-anisyl, 2-benzoylnaphthalene, 4,4′-bis(dimethylamino)benzophenone, 4,4′-dichlorobenzophenone and 1,4-dibenzoylbenzene, anthraquinone-based or thioxanthone-based compounds such as 2-ethylanthraquinone, 2-isopropylthioxanthone, 2-chlorothioxanthone, 2,4-dimethylthioxanthone, 2,4-diethylthioxanthone and 2,4-dichlorothioxanthone and the like.

Examples of the other photopolymerization initiators include camphor quinone, 1-phenyl-1,2-propanedione-2-(o-ethoxycarbonyl)oxime, acridine-based compounds, triazine-based compounds and imidazole-based compounds.

To efficiently absorb light energy and generate a radical, the photopolymerization initiator preferably has an absorption wavelength in the wavelength range of the light source used for the light application. In particular, an acylphosphine oxide-based compound having an absorption wavelength at a relatively long wavelength side is preferably contained.

To compensate for the curability of the outermost layer surface, an acylphosphine oxide-based compound and an initiator of the hydrogen extraction type are further preferably used in combination. Here, the ratio of the initiator of the hydrogen extraction type to the acylphosphine oxide-based compound is not particularly limited. The ratio to one part by mass of the acylphosphine oxide-based compound is preferably 0.1 parts by mass or more to compensate for the surface curability, and the ratio is preferably five parts by mass or less to maintain the internal curability.

Moreover, one having the effect of promoting photopolymerization can be used alone or in combination with the photopolymerization initiator. Examples of the one having the effect of promoting photopolymerization include triethanolamine, methyldiethanolamine, ethyl 4-dimethylaminobenzoate, isoamyl 4-dimethylaminobenzoate, (2-dimethylamino)ethyl benzoate, 4,4′-dimethylaminobenzophenone and the like.

One kind of polymerization initiator or a mixture of two or more kinds thereof may be used. The polymerization initiator content, based on 100 parts by mass of the total material having radically polymerizable property, is preferably 0.5 to 40 parts by mass, in particular, further preferably one part by mass or more or 20 parts by mass or less.

### (Inorganic Particles)

The protective layer of the present invention may contain inorganic particles to improve the strong exposure property or the mechanical strength or to add electron-transporting capability. However, inorganic particles do not have to be contained.

In the present invention, that inorganic particles do not have to be contained because the protective layer contains a specific electron-transporting compound is one of the characteristics of the present invention.

Examples of the inorganic particles include a metal powder, a metal oxide, a metal fluoride, potassium titanate, boron nitride and the like, and any inorganic particles which can be generally used for an electrophotographic photoreceptor can be used.

As the inorganic particles, particles of one kind may be used alone, or particles of two or more kinds may also be mixed and used.

### (Other Materials)

The protective layer of the present invention may contain another material according to the need. Examples of the other material include a stabilizer (a thermal stabilizer, an ultraviolet absorber, a light stabilizer, an antioxidant or the like), a dispersant, an antistatic agent, a colorant, a lubricant and the like. One kind thereof alone or two or more kinds thereof at any ratio in any combination can be appropriately used.

### (Formation Method of Protective Layer of Present Invention)

### [Coating Liquid for Forming Protective Layer]

The protective layer of the present invention can be formed, for example, by applying a coating liquid (referred to as “the coating liquid for forming the protective layer of the present invention”) which is obtained by dissolving or dispersing a curable composition containing a curable compound, a polymerization initiator and an electron-transporting compound and containing inorganic particles and another material according to the need in a solvent or in a dispersant, on the photosensitive layer of the present invention and curing the coating liquid. However, the method is not limited to the method.

Here, when the electron-transporting compound has a chain-polymerizable functional group such as an acrylamide group, a methacrylamide group, an acryloyl group or a methacryloyl group, the electron-transporting compound can also play the role of the curable compound. In this case, another curable compound does not have to be contained in addition to the electron-transporting compound having a chain-polymerizable functional group. Even when no curable compound is contained or when the curable compound content is low, because the electron-transporting compound having a chain-polymerizable functional group is used, the mechanical strength of the protective layer is sufficiently obtained, and the deterioration in the residual potential due to the addition of the curable compound can be suppressed. This, however, does not exclude the combination use of the electron-transporting compound having a chain-polymerizable functional group and the curable compound.

The electron-transporting compound used for the coating liquid for forming the protective layer of the present invention is preferably the compound represented by the formula (1).

Preferable aspects of the curable compound, the polymerization initiator, the inorganic particles and the other material used for the coating liquid for forming the protective layer of the present invention are the same as those of the respective materials used for the protective layer of the present invention.

The ratio of the curable compound to the electron-transporting compound (curable compound/electron-transporting compound) in the coating liquid for forming the protective layer of the present invention is the same as the ratio of the curable compound to the electron-transporting compound (curable compound/electron-transporting compound) in the protective layer of the present invention described above.

In view of the membrane uniformity of the protective layer, the electron-transporting compound content of the coating liquid for forming the protective layer of the present invention, based on 100 parts by mass of the solvent, is preferably four parts by mass or more, more preferably six parts by mass or more, further preferably eight parts by mass or more. On the other hand, in view of the solubility, the content based on 100 parts by mass of the solvent is preferably 14 parts by mass or less, more preferably 12 parts by mass or less, further preferably 10 parts by mass or less.

In view of the membrane uniformity of the protective layer, the curable compound content of the coating liquid for forming the protective layer of the present invention, based on 100 parts by mass of the solvent, is preferably one part by mass or more, more preferably two parts by mass or more, further preferably four parts by mass or more. On the other hand, in view of the solubility, the content based on 100 parts by mass of the solvent is preferably 10 parts by mass or less, more preferably eight parts by mass or less, further preferably six parts by mass or less.

In particular, when the electron-transporting compound contained in the coating liquid for forming the protective layer of the present invention has a chain-polymerizable functional group, in view of the residual potential, the curable compound content of the coating liquid for forming the protective layer of the present invention, based on 100 parts by mass of the solvent, is preferably 10 parts by mass or less, more preferably five parts by mass or less, further preferably 0 part by mass.

As the solvent used for the coating liquid for forming the protective layer of the present invention, for example, an organic solvent can be used. Examples of the organic solvent include: alcohols such as methanol, ethanol, propanol and 2-methoxyethanol; ethers such as tetrahydrofuran, 1,4-dioxane and dimethoxyethane; esters such as methyl formate and ethyl acetate; ketones such as acetone, methylethylketone and cyclohexanone; aromatic hydrocarbons such as benzene, toluene, xylene and anisole; chlorinated hydrocarbons such as dichloromethane, chloroform, 1,2-dichloroethane, 1,1,2-trichloroethane, 1,1,1-trichloroethane, tetrachloroethane, 1,2-dichloropropane and trichloroethylene; nitrogen-containing compounds such as n-butylamine, isopropanolamine, diethylamine, triethanolamine, ethylene diamine and triethylene diamine; aprotic polar solvents such as acetonitrile, N-methylpyrrolidone, N,N-dimethylformamide and dimethylsulfoxide; and the like. A mixed solvent thereof in any combination at any ratio can also be used. Of these, in view of the solubility and the coatability, the solvent is preferably an alcohol, an ether, an aromatic hydrocarbon or an aprotic polar solvent, more preferably an alcohol, an ether or an aromatic hydrocarbon, further preferably an alcohol or an ether, most preferably an alcohol.

Moreover, even when the materials for the protective layer of the electrophotographic photoreceptor of the present invention do not dissolve in an organic solvent itself, the organic solvent can be used, for example, when the materials can dissolve in a mixed solvent with the above organic solvent. In general, coating unevenness can be reduced when a mixed solvent is used. When a dip coating method is used in the coating method described below, a solvent in which the under layer does not dissolve is preferably selected. From this point, an alcohol is particularly preferably contained.

The ratio of the organic solvent used for the coating liquid for forming the protective layer of the present invention and the solid content depends on the coating method of the coating liquid for forming the protective layer and can be appropriately changed and used in such a manner that a uniform coating film is formed by the coating method applied.

### [Coating Method]

The method for coating the coating liquid for forming the protective layer of the present invention is not particularly limited, and examples thereof include a spray coating method, a spiral coating method, a ring coating method, a dip coating method and the like.

After forming a coating film by the coating method, the coating film is dried. At this point, when required sufficient drying can be achieved, the temperature and the period of drying are not limited. When the protective layer is applied after air drying alone after applying the photosensitive layer, however, sufficient drying is preferably conducted by the method described in the method for forming the photosensitive layer described below.

### [Curing Method of Protective Layer of Present Invention]

The protective layer of the present invention can be formed by applying the coating liquid for forming the protective layer of the present invention and then curing by applying energy from outside. The external energy used here may be heat, light or radiation.

The method for applying heat energy may be a heating method using air, gas such as nitrogen, steam, various heating media, infrared ray or electromagnetic waves. Moreover, the heating can be conducted from the coated surface side or from the support side. The heating temperature is preferably 100° C. or higher and 170° C. or lower.

As the light energy, a UV light source such as a high-pressure mercury lamp, a metal halide lamp, an electrodeless lamp bulb and a light-emitting diode, which have emission wavelengths mainly of ultraviolet light (UV), can be used. Moreover, a visible light source can also be selected corresponding to the absorption wavelength of the chain-polymerizable compound or the photopolymerization initiator.

In view of the curability, the light irradiation amount is preferably 10 J/cm² or more, further preferably 30 J/cm² or more, particularly preferably 100 J/cm² or more. In view of the electrical property, the light irradiation amount is preferably 500 J/cm² or less, further preferably 300 J/cm² or less, particularly preferably 200 J/cm² or less.

The radiation energy may be one using electron beam (EB).

Of the energy types, in view of the easiness of the regulation of the reaction speed, the simpleness of the device and the length of the pot life, light energy is preferably used.

After curing the protective layer, to reduce the residual stress, reduce the residual radical or improve the electrical property, a heating step may be added. The heating temperature is preferably 60° C. or higher, more preferably 100° C. or higher, and is preferably 200° C. or lower, more preferably 150° C. or lower.

### (Layer Thickness)

In view of the abrasion resistance, the thickness of the protective layer of the present invention is preferably 0.5 μm or more, in particular, further preferably 1 μm or more. On the other hand, in view of the electrical property, the thickness is preferably 5 μm or less, in particular, further preferably 3 μm or less.

In view of the same points, the thickness of the protective layer of the present invention is preferably 1/50 or more of the thickness of the photosensitive layer of the present invention, in particular, more preferably 1/40 or more, in particular, further preferably 1/30 or more. On the other hand, the thickness is preferably ⅕ or less, in particular, more preferably 1/10 or less, in particular, further preferably 1/20 or less.

### <Photosensitive Layer of Present Invention>

The photosensitive layer (also referred to as “the photosensitive layer of the present invention”) in the electrophotographic photoreceptor of the present invention is a layer containing at least a charge generation material (CGM) and a charge transport material.

The photosensitive layer of the present invention may be a single-layer photosensitive layer containing both a charge generation material and a charge transport material in the same layer or may be a laminate-type photosensitive layer which is separated into a charge generation layer and a charge transport layer.

### <Single-Layer Photosensitive Layer>

When the photosensitive layer of the present invention is a single-layer photosensitive layer, at least a charge generation material (CGM), a hole transport material (HTM) and an electron transport material (ETM) and a binder resin are preferably contained in the same layer.

### (Charge Generation Material)

As the charge generation material used for the photosensitive layer of the present invention, for example, various photoconductive materials such as inorganic photoconductive materials and organic pigments can be used. Of these, in particular, an organic pigment is preferable, and a phthalocyanine pigment and an azo pigment are more preferable.

In particular, when a phthalocyanine pigment is used as the charge generation material, specifically, a non-metal phthalocyanine, a phthalocyanine in which a metal such as copper, indium, gallium, tin, titanium, zinc, vanadium, silicon and germanium, an oxide thereof, a halogenated material thereof or the like is coordinated or the like is used. Of these, X-form or τ-form non-metal phthalocyanine, A-form, B-form, D-form or another form titanyl phthalocyanine, vanadyl phthalocyanine, chloroindium phthalocyanine, chlorogallium phthalocyanine, hydroxygallium phthalocyanine and the like, which have high sensitivity, are suitable.

Moreover, when an azo pigment is used, various known bisazo pigments and trisazo pigments are suitably used.

One kind of charge generation material may be used alone, or two or more kinds thereof in any combination at any ratio may be used in combination. When two or more kinds of charge generation material are used in combination, as the method for mixing the charge generation materials used in combination, the charge generation materials may be mixed later and used or mixed in the production/processing steps of the charge generation materials such as synthesis, pigment formation and crystallization and used.

In view of the electrical property, the particle size of the charge generation material is desirably small. Specifically, the particle size of the charge generation material is preferably 1 μm or less, more preferably 0.5 μm or less. The lower limit is 0.01 μm or more. Here, the particle size of the charge generation material means the particle size in the state of being contained in the photosensitive layer.

Furthermore, in view of the sensitivity, the amount of the charge generation material in the single-layer photosensitive layer is preferably 0.1 mass % or more, more preferably 0.5 mass % or more. Moreover, in view of the sensitivity and the electrostatic property, the amount is preferably 50 mass % or less, more preferably 20 mass % or less.

### (Charge Transport Material)

Charge transport materials are classified into hole transport materials mainly having hole transport capability and electron transport materials mainly having electron transport capability. However, when the photosensitive layer of the present invention is a single-layer photosensitive layer, at least a hole transport material and an electron transport material are preferably contained in the same layer.

### [Hole Transport Material]

The hole transport material (HTM) can be selected from known materials and used. Examples thereof include electron-donating materials, such as heterocyclic compounds including a carbazole derivative, an indole derivative, an imidazole derivative, an oxazole derivative, a pyrazole derivative, a thiadiazole derivative, a benzofuran derivative and the like, an aniline derivative, a hydrazone derivative, an arylamine derivative, a stilbene derivative, a butadiene derivative, an enamine derivative, a material in which two or more kinds of the compounds are bound and a polymer having a group derived from such a compound in the main chain or a side chain, and the like.

Of these, a carbazole derivative, an arylamine derivative, a stilbene derivative, a butadiene derivative, an enamine derivative and a material in which two or more kinds of the compounds are bound are preferable, and an arylamine derivative and an enamine derivative are more preferable.

One kind of hole transport material may be used alone, or two or more kinds thereof may be used at any ratio in any combination.

The structures of preferable hole transport materials are exemplified below.

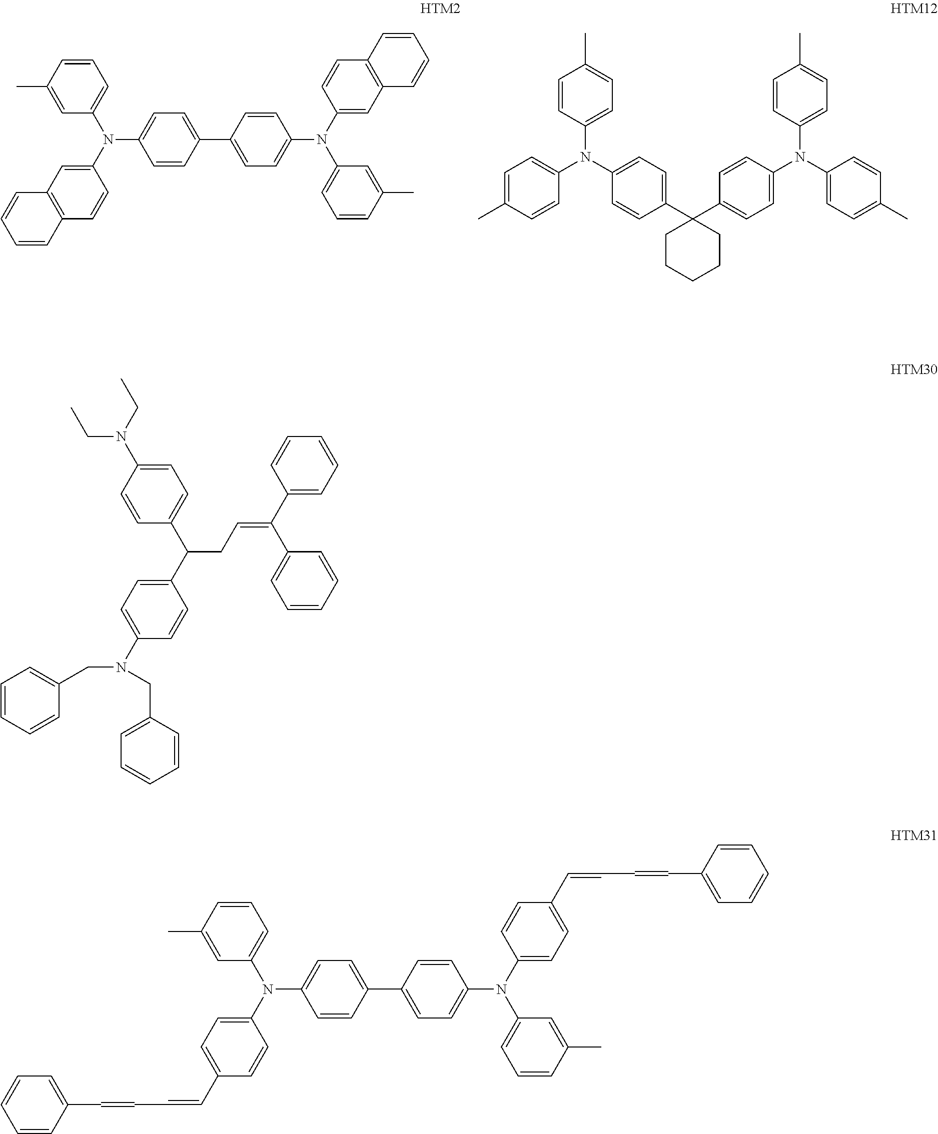

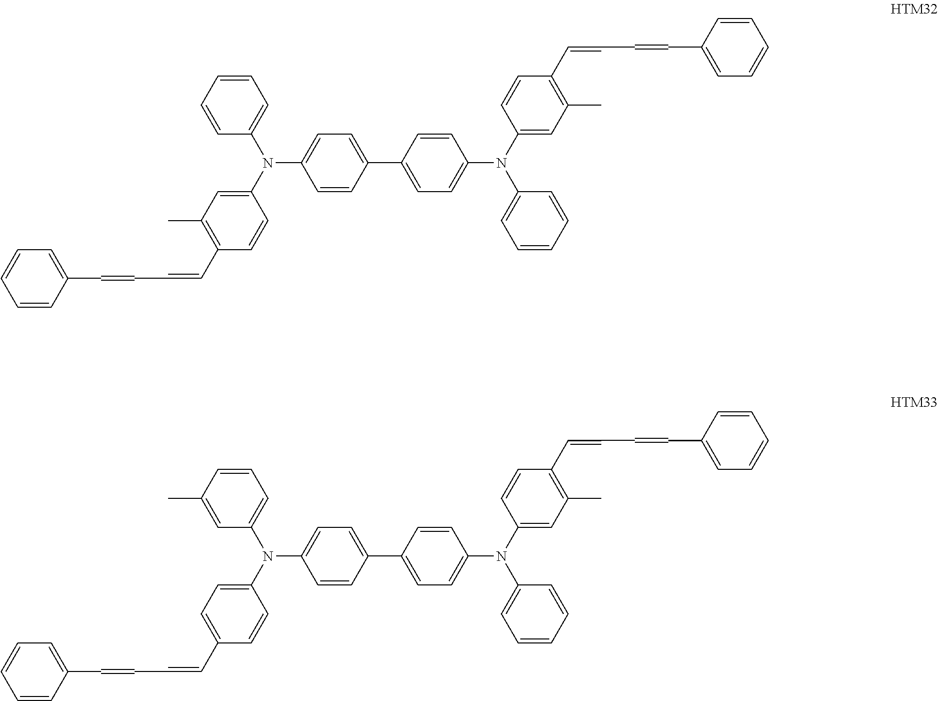 [SMILES: Cc1cc(N(c2ccccc2)c2ccc(-c3ccc(N(c4ccccc4)c4ccc(C=CC=Cc5ccccc5)c(C)c4)cc3)cc2)ccc1C=CC=Cc1ccccc1 ; Cc1cccc(N(c2ccc(C=CC=Cc3ccccc3)cc2)c2ccc(-c3ccc(N(c4ccccc4)c4ccc(C=CC=Cc5ccccc5)c(C)c4)cc3)cc2)c1]

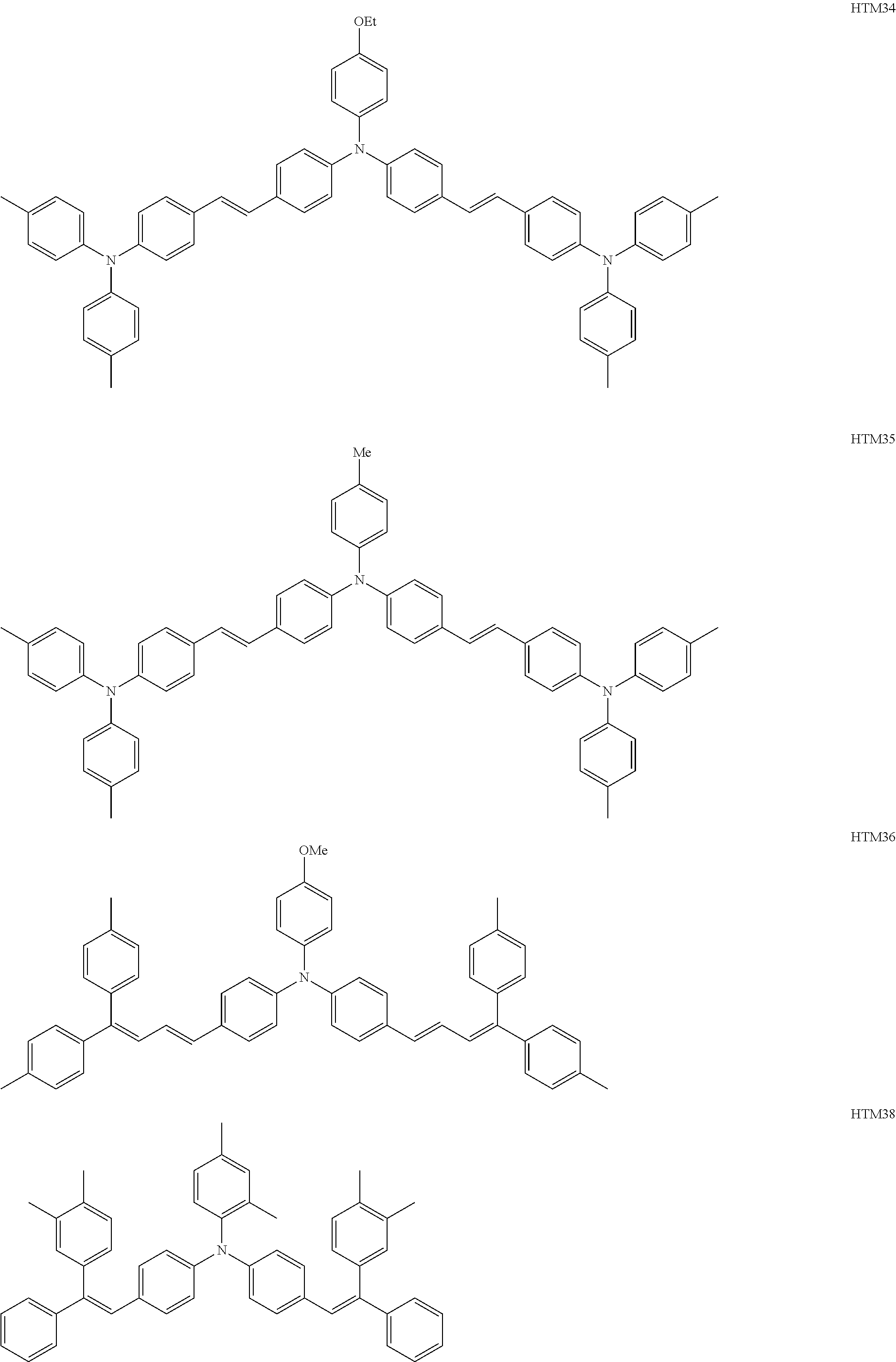

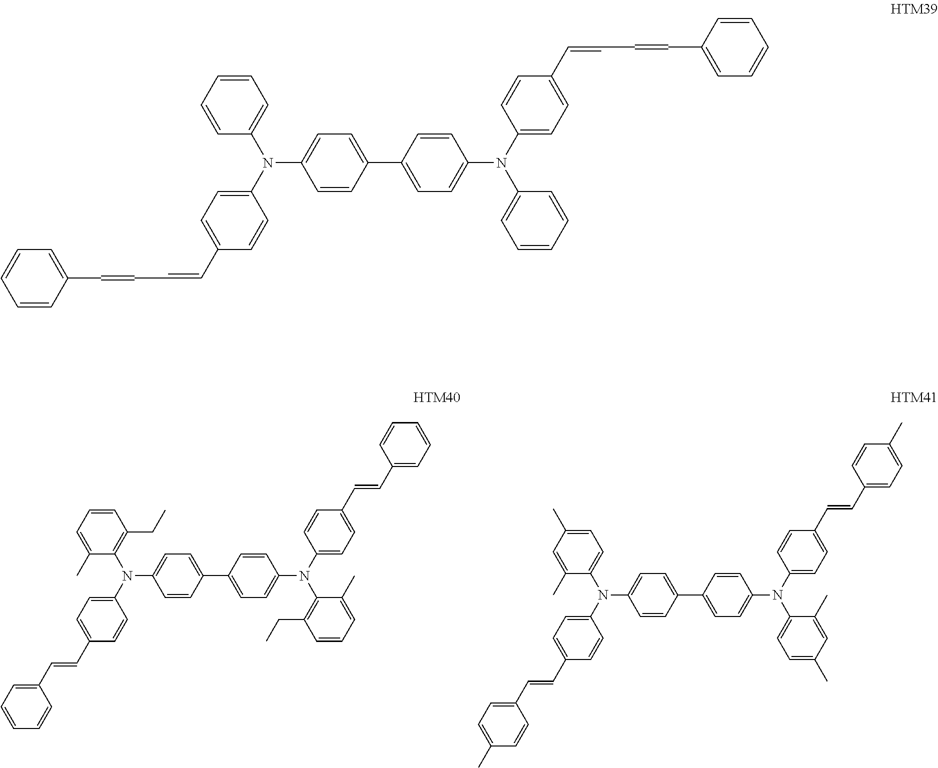 [SMILES: C(C=Cc1ccc(N(c2ccccc2)c2ccc(-c3ccc(N(c4ccccc4)c4ccc(C=CC=Cc5ccccc5)cc4)cc3)cc2)cc1)=Cc1ccccc1 ; CCc1cccc(C)c1N(c1ccc(/C=C/c2ccccc2)cc1)c1ccc(-c2ccc(N(c3ccc(/C=C/c4ccccc4)cc3)c3c(C)cccc3CC)cc2)cc1 ; Cc1ccc(/C=C/c2ccc(N(c3ccc(-c4ccc(N(c5ccc(/C=C/c6ccc(C)cc6)cc5)c5ccc(C)cc5C)cc4)cc3)c3ccc(C)cc3C)cc2)cc1]

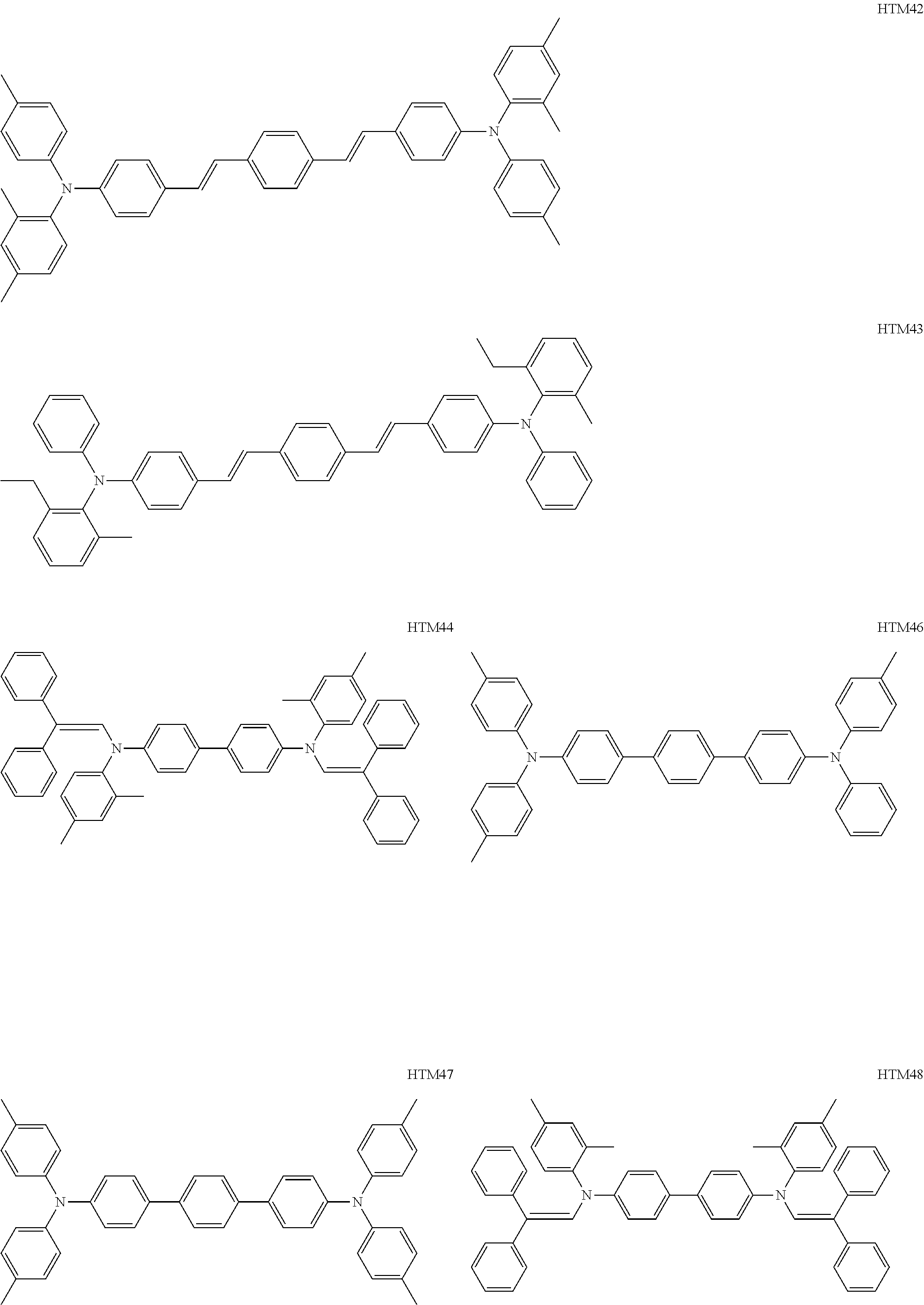

Of the hole transport materials, in terms of the electrical property, HTM31, HTM32, HTM33, HTM34, HTM35, HTM39, HTM40, HTM41, HTM42, HTM43 and HTM48 are preferable, and HTM39, HTM40, HTM41, HTM42, HTM43 and HTM48 are further preferable.

### [Electron Transport Material]

The electron transport material (ETM) can be selected from known materials and used. Examples thereof include electron-withdrawing materials including aromatic nitro compounds such as 2,4,7-trinitrofluorenone, cyano compounds such as tetracyanoquinodimethane, quinone compounds such as diphenoquinone and the like, known cyclic ketone compounds, perylene pigments (perylene derivatives) and the like. Of these, in view of the electrical property, quinone compounds and perylene pigments (perylene derivatives) are preferable, and quinone compounds are more preferable.

Of the quinone compounds, in view of the electrical property, diphenoquinone or dinaphthylquinone is preferable. Of these, dinaphthylquinone is more preferable.

One kind of electron transport material may be used alone, or two or more kinds thereof may be used at any ratio in any combination.

The structures of preferable electron transport materials are exemplified below.

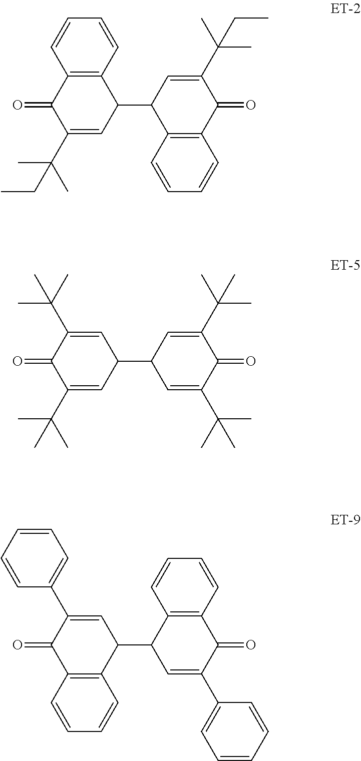 [SMILES: CCC(C)(C)C1=CC(C2C=C(C(C)(C)CC)C(=O)c3ccccc32)c2ccccc2C1=O ; CC(C)(C)C1=CC(C2C=C(C(C)(C)C)C(=O)C(C(C)(C)C)=C2)C=C(C(C)(C)C)C1=O ; O=C1C(c2ccccc2)=CC(C2C=C(c3ccccc3)C(=O)c3ccccc32)c2ccccc21]

Of the electron transport materials, in terms of the electrical property, ET-2 and ET-5 are preferable, and ET-2 is further preferable.

### (Binder Resin)

Next, the binder resin used for the photosensitive layer of the present invention will be explained.

Examples of the binder resin used for the photosensitive layer of the present invention include: vinyl polymers such as polymethylmethacrylate, polystyrene and polyvinyl chloride or copolymers thereof; vinyl alcohol resins; polyvinyl butyral resins; polyvinyl formal resins; partially modified polyvinyl acetal resins; polyarylate resins; polyamide resins; polyurethane resins; polycarbonate resins; polyester resins; polyester carbonate resins; polyimide resins; phenoxy resins; epoxy resins; silicone resins; and partially cross-linked cured materials thereof. The resins may be modified with a silicon reagent or the like. One kind thereof may be used alone, or two or more kinds thereof can be used at any ratio in any combination.

Moreover, the binder resin used for the photosensitive layer of the present invention preferably contains one kind or two or more kinds of polymer obtained by interfacial polymerization.

The binder resin obtained by interfacial polymerization is preferably a polycarbonate resin or a polyester resin, in particular, preferably a polycarbonate resin or a polyarylate resin. Moreover, in particular, a polymer obtained from an aromatic diol as a raw material is preferable.

### (Other Substances)

In addition to the above materials, the photosensitive layer of the present invention may contain a known additive such as an antioxidant, a plasticizer, an ultraviolet absorber, an electron-withdrawing compound, a leveling agent and a visible light-shielding agent to improve the film forming property, the flexibility, the coatability, the contamination resistance, the gas resistance, the light resistance or the like. Moreover, the photosensitive layer of the present invention may contain various additives such as a sensitizer, a dye, a pigment (excluding those which are the charge generation material, the hole transport material and the electron transport material listed above) and a surfactant according to the need. Examples of the surfactant include silicone oil, a fluorine-based compound and the like. In the present invention, one kind thereof alone or two or more kinds thereof at any ratio in any combination can be appropriately used.

Moreover, for the purpose of reducing the friction resistance on the surface of the photosensitive layer, the photosensitive layer may contain a fluorine-based resin, a silicone resin or the like or may contain particles of such a resin or particles of an inorganic compound such as aluminum oxide.

### (Layer Thickness)

When the photosensitive layer of the present invention is a single-layer photosensitive layer, in view of the dielectric breakdown resistance, the thickness of the photosensitive layer of the present invention is preferably 20 μm or more, in particular, further preferably 25 μm or more. On the other hand, in view of the electrical property, the thickness is preferably 50 μm or less, in particular, further preferably 40 μm or less.

### <Laminate-Type Photosensitive Layer>

When the electrophotographic photoreceptor of the present invention is a laminate-type photosensitive layer, the electrophotographic photoreceptor can have a configuration obtained, for example, by laminating a charge transport layer (CTL) containing a charge transport material on a charge generation layer (CGL) containing a charge generation material (CGM). At this point, a layer other than the charge generation layer (CGL) and the charge transport layer (CTL) can also be included.

### <Charge Generation Layer (CGL)>

The charge generation layer generally contains a charge generation material (CGM) and a binder resin.

The charge generation material (CGM) and the binder resin are the same as those explained for the single-layer photosensitive layer.

### (Other Components)

The charge generation layer can contain another component according to the need in addition to the charge generation material and the binder resin. For example, for the purpose of improving the film forming property, the flexibility, the coatability, the contamination resistance, the gas resistance, the light resistance or the like, a known additive such as an antioxidant, a plasticizer, an ultraviolet absorber, an electron-withdrawing compound, a leveling agent, a visible light-shielding agent and a filler may be contained.

### (Mixing Ratio)

When the proportion of the charge generation material is too high in the charge generation layer, the stability of the coating liquid may decrease due to the cohesion of the charge generation material or the like, while the sensitivity as the photoreceptor may decrease when the proportion of the charge generation material is too low. Thus, regarding the mixing ratio (mass) of the binder resin and the charge generation material, based on 100 parts by mass of the binder resin, the charge generation material is contained preferably at 10 parts by mass or more, in particular, more preferably at 30 parts by mass or more, while the charge generation material is contained preferably at a ratio of 1000 parts by mass or less, in particular, further preferably at a ratio of 500 parts by mass or less, more preferably at a ratio of 300 parts by mass or less in view of the membrane strength, further preferably at a ratio of 200 parts by mass or less.

### (Layer Thickness)

The thickness of the charge generation layer is preferably 0.1 μm or more, in particular, further preferably 0.15 μm or more. On the other hand, the thickness is preferably 10 μm or less, in particular, further preferably 0.6 μm or less.

### <Charge Transport Layer (CTL)>

The charge transport layer (CTL) generally contains a charge transport material and a binder resin.

The charge transport material and the binder resin are the same as those explained for the single-layer photosensitive layer.

In the charge transport layer (CTL), regarding the mixing ratio of the binder resin and the hole transport material (HTM), based on 100 parts by mass of the binder resin, the hole transport material (HTM) is preferably blended at a ratio of 20 parts by mass or more, in particular, more preferably at a ratio of 30 parts by mass or more to reduce the residual potential, further preferably at a ratio of 40 parts by mass or more in view of the stability during repeated use and the charge transfer degree. On the other hand, in view of the thermal stability of the photosensitive layer, based on 100 parts by mass of the binder resin, the hole transport material (HTM) is preferably blended at a ratio of 200 parts by mass or less, more preferably at a ratio of 150 parts by mass or less in view of the compatibility of the hole transport material (HTM) and the binder resin, particularly preferably at a ratio of 120 parts by mass or less in view of the glass transition temperature.

### (Other Components)

The charge transport layer can contain another component according to the need in addition to the electron transport material (ETM), the hole transport material (HTM) and the binder resin. For example, for the purpose of improving the film forming property, the flexibility, the coatability, the contamination resistance, the gas resistance, the light resistance or the like, a known additive such as an antioxidant, a plasticizer, an ultraviolet absorber, an electron-withdrawing compound, a leveling agent, a visible light-shielding agent and a filler may be contained.

### (Layer Thickness)

The layer thickness of the charge transport layer is not particularly restricted. In view of the electrical property and the image stability and in view of the high resolution, the thickness is preferably 5 μm or more and 50 μm or less, in particular, more preferably 10 μm or more or 35 μm or less, in particular, further preferably 15 μm or more or 25 μm or less.

### <Formation Method of Photosensitive Layer>

Both for the laminate-type and the single layer, each layer can be formed as follows.

The layers can be formed one by one by repeating coating/drying steps of coating liquids obtained by dissolving or dispersing the materials to be contained in a solvent on a conductive support by a known method such as dip coating, spray coating, nozzle coating, bar coating, roll coating and blade coating.

The forming method, however, is not limited to such a method.

The solvent or the dispersant used for producing the coating liquids is not particularly restricted. Specific examples thereof include an alcohol, an ether, an aromatic hydrocarbon, a chlorinated hydrocarbon and the like. One kind thereof may be used alone, or two or more kinds thereof in any combination of any kinds may be used in combination.

The amount of the solvent or the dispersant used is not particularly restricted. Considering the purposes of the layers and the properties of the solvent/dispersant selected, the amount is preferably appropriately adjusted in such a manner that the solid concentrations of the coating liquids and the physical properties such as the viscosity are in the desired ranges.

The coating films are preferably dried by drying to the touch at room temperature and then heat drying generally in a temperature range of 30° C. or higher and 200° C. or lower for one minute to two hours in still state or with ventilation. The heating temperature may be constant, or heating may be conducted while changing the temperature during drying.

### <Conductive Support of Present Invention>

The conductive support (also referred to as “the conductive support of the present invention”) of the electrophotographic photoreceptor of the present invention is not particularly limited as long as the conductive support supports the layers formed thereon and exhibits conductivity.

As the conductive support of the present invention, for example, a metal material such as aluminum, an aluminum alloy, stainless steel, copper and nickel, a resin material to which conductivity is added by causing conductive powder such as a metal, carbon and tin oxide to coexist, a resin material, glass material or paper material having a surface on which a conductive material such as aluminum, nickel and ITO (indium oxide-tin oxide alloy) is deposited or applied or the like can be mainly used.

Regarding the form of the conductive support of the present invention, a drum, a cylinder, a sheet, a belt or the like is used.

The conductive support of the present invention may be obtained by applying a conductive material having appropriate resistance on a conductive support made of a metal material to regulate the conductivity, the surface property or the like or to cover the defect.

When a metal material such as an aluminum alloy is used as the conductive support of the present invention, the metal material used may be coated with an anodized film.

The average thickness of the anodized film is preferably 20 μm or less, in particular, further preferably 7 μm or less.

When the metal material is coated with an anodized film, sealing treatment is preferably conducted. The sealing treatment can be conducted by a known method.

The surface of the conductive support of the present invention may be smooth or may be roughened using a special cutting method or by subjecting to grinding. Moreover, the surface may be roughened by mixing particles having an appropriate particle size in the material composing the support.

Here, the undercoat layer explained below may be provided between the conductive support of the present invention and the photosensitive layer to improve the adhesion, the blocking property or the like.

### <Undercoat Layer of Present Invention>

The electrophotographic photoreceptor of the present invention may have an undercoat layer (also referred to as “the undercoat layer of the present invention”) between the photosensitive layer of the present invention and the conductive support of the present invention.

As the undercoat layer of the present invention, for example, a resin, a material obtained by dispersing particles of an organic pigment, a metal oxide or the like in a resin or the like can be used.

Examples of the organic pigment used for the undercoat layer of the present invention include a phthalocyanine pigment, an azo pigment, a perylene pigment and the like. Of these, a phthalocyanine pigment or an azo pigment, specifically, the phthalocyanine pigment and the azo pigment used as the charge generation material described above, can be used.

Examples of the metal oxide particles used for the undercoat layer of the present invention include metal oxide particles containing one kind of metal element such as titanium oxide, aluminum oxide, silicon oxide, zirconium oxide, zinc oxide and iron oxide and metal oxide particles containing two or more metal elements such as calcium titanate, strontium titanate and barium titanate. One kind of particles may be used alone for the undercoat layer, or more than one kind of particles at any ratio in any combination may be mixed and used.

Of the metal oxide particles, titanium oxide and aluminum oxide are preferable, and in particular, titanium oxide is preferable.

The particle size of the metal oxide particles used for the undercoat layer of the present invention is not particularly limited. Due to the characteristics of the undercoat layer and the stability of the solution for forming the undercoat layer, the average primary particle size is preferably 10 nm or more and is 100 nm or less, more preferably 50 nm or less.

The binder resin used for the undercoat layer of the present invention can be selected, for example, from the following materials and used: a polyvinyl acetal-based resin such as a polyvinyl butyral resin; an insulating resin such as a polyarylate resin, a polycarbonate resin, a polyester resin, a phenoxy resin, an acrylic resin, a methacrylic resin, a polyamide resin, a polyurethane resin, an epoxy resin, a silicone resin, a polyvinyl alcohol resin and a styrene-alkyd resin; and the like. In this regard, however, the binder resin is not limited to these polymers. One kind of the binder resins may be used alone, or two or more kinds thereof may be mixed and used. The binder resin may be used in the form cured with a curing agent.

Of these, a polyvinyl acetal-based resin, an alcohol-soluble copolymerized polyamide, a modified polyamide and the like exhibit excellent dispersibility and coatability and thus are preferable. Of these, an alcohol-soluble copolymerized polyamide is particularly preferable.

The mixing ratio of the particles to the binder resin can be selected freely. It is preferable to use in the range of 10 mass % to 500 mass % in terms of the stability and the coatability of the dispersion.

The thickness of the undercoat layer of the present invention can be selected freely. From the characteristics of the electrophotographic photoreceptor and the coatability of the dispersion, the thickness is preferably 0.1 μm or more and is further preferably 20 μm or less. Moreover, the undercoat layer may contain a known antioxidant or the like.

### <Other Layers>

Moreover, the electrophotographic photoreceptor of the present invention may appropriately have another layer according to the need in addition to the conductive support of the present invention, the photosensitive layer of the present invention, the protective layer of the present invention and the undercoat layer of the present invention described above.

### <Physical Properties of Electrophotographic Photoreceptor of Present Invention>

The electrophotographic photoreceptor of the present invention can have the following physical properties.

### (Martens Hardness)

To achieve practically sufficient abrasion resistance, the Martens hardness of the electrophotographic photoreceptor of the present invention is preferably 75 N/mm² or more, more preferably 150 N/mm² or more, further preferably 200 N/mm² or more, in particular, 215 N/mm² or more, in particular, particularly preferably 220 N/mm² or more.

In the present invention, the Martens hardness of the photoreceptor means the Martens hardness measured from the surface side of the photoreceptor.

The Martens hardness can be measured by the method described in the Examples below.

### (Elastic Deformation Ratio)

To achieve practically sufficient abrasion resistance, the elastic deformation ratio of the electrophotographic photoreceptor of the present invention is preferably 14% or more, more preferably 25% or more, further preferably 30% or more, particularly preferably 32% or more.

In the present invention, the elastic deformation ratio of the photoreceptor means the elastic deformation ratio measured from the surface side of the photoreceptor.

The elastic deformation ratio can be measured by the method described in the Examples below.

### <<Image Formation Device of Present Invention>>

Using the electrophotographic photoreceptor of the present invention, an image formation device (“the image formation device of the present invention”) can be configured.

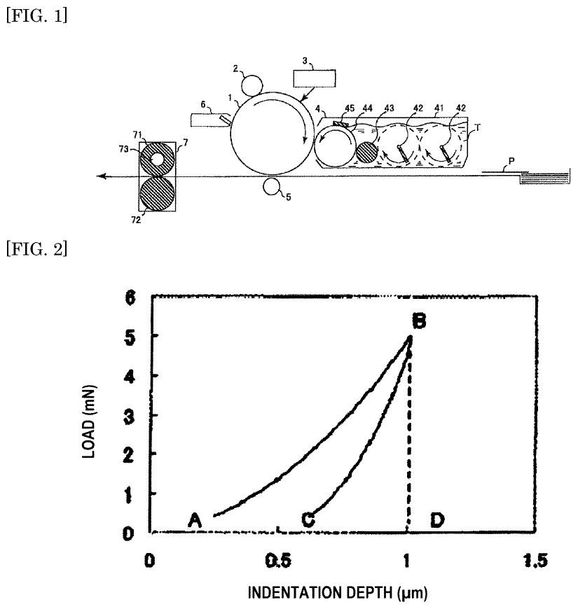

As illustrated in FIG. 1, the image formation device of the present invention is configured with an electrophotographic photoreceptor 1 of the present invention, a charging device 2, an exposure device 3 and a developing device 4, and according to the need, a transfer device 5, a cleaning device 6 and a fixing device 7 are further provided.

The electrophotographic photoreceptor 1 of the present invention is not particularly restricted as long as it is the electrophotographic photoreceptor of the present invention described above. As an example thereof, FIG. 1 illustrates a drum-type photoreceptor in which the photosensitive layer described above is formed on the surface of a cylindrical conductive support. The charging device 2, the exposure device 3, the developing device 4, the transfer device 5 and the cleaning device 6 are arranged along the peripheral surface of the electrophotographic photoreceptor 1 of the present invention.

The charging device 2 can be a non-contact corona charging device such as a corotron and a scorotron or a contact-type charging device (a direct charging device) for charging by bringing a charged material to which voltage is applied into contact with the photoreceptor surface. Examples of the contact charging device include a charging roller, a charging brush and the like. In FIG. 1, a roller-type charging device (charging roller) is illustrated as an example of the charging device 2.

The type of the exposure device 3 is not particularly restricted as long as it can expose the electrophotographic photoreceptor 1 of the present invention and form an electrostatic latent image on the photosensitive surface of the electrophotographic photoreceptor 1 of the present invention.

Moreover, exposure may be conducted by a photoreceptor internal exposure method. The light for the exposure may be any light.

The type of a toner T may be any type, and in addition to a powdery toner, a polymerized toner using a suspension polymerization method, an emulsion polymerization method or the like and the like can be used.

The configuration of the developing device 4 may also be any configuration. The developing device 4 illustrated in FIG. 1 has a configuration in which the toner T is made into a thin layer with a regulating member (a developing blade) 45, charged by friction to predetermined polarity, transferred while supported on a developing roller 44 and brought into contact with the surface of the photoreceptor 1. The configuration, however, is not limited to the configuration.

The type of the transfer device 5 is not particularly restricted, and a device using any type such as an electrostatic image transfer method including corona transfer, roller transfer, belt transfer and the like, a pressure transfer method and an adhesion transfer method can be used.

The cleaning device 6 is not particularly restricted. For example, any cleaning device such as a brush cleaner, a magnetic roller cleaner and a blade cleaner can be used. When a small amount of the toner or almost no toner remains on the surface of the photoreceptor, the cleaning device 6 does not have to be provided.

The fixing device 7 may have any configuration.

Here, the image formation device may have a configuration which can conduct, for example, a charge elimination step in addition to the configuration described above.

Moreover, the image formation device may be configured with further modification and may have, for example, a configuration which can conduct a step such as a pre-exposure step and a supplemental charging step, a configuration for conducting offset printing or a configuration of the full-color tandem type using more than one kind of toner.

### <<Electrophotographic Cartridge of Present Invention>>

The electrophotographic photoreceptor 1 of the present invention can be configured as an integrated cartridge (referred to as “the electrophotographic cartridge of the present invention”) by combining with one or two or more of the charging device 2, the exposure device 3, the developing device 4, the transfer device 5, the cleaning device 6 and the fixing device 7.

The electrophotographic cartridge of the present invention can have a configuration which can be attached to and detached from the main body of an electrophotographic device such as a copier and a laser beam printer. In this case, for example, when the electrophotographic photoreceptor 1 of the present invention or another member is deteriorated, by removing the electrophotographic photoreceptor cartridge from the main body of the image formation device and installing a new electrophotographic photoreceptor cartridge into the main body of the image formation device, maintenance and the management of the image formation device become easy.

### <<Explanation of Terms>>

In the present invention, the expression “X to Y” (X and Y are numbers) includes the meaning of “X or more and Y or less” and the meaning of “preferably larger than X” or “preferably smaller than Y” unless otherwise specified.

Moreover, the expression “X or more” (X is a number) or “Y or less” (Y is a number) also includes the meaning of “preferably larger than X” or “preferably less than Y”.

### EXAMPLES

Embodiments of the present invention will be explained further specifically with Examples below. However, the following Examples are for explaining the present invention in detail, and the present invention is not limited to the Examples shown below and can be carried out with any modification unless departing from the gist thereof. Moreover, the “parts” in the Examples and the Comparative Examples below are “parts by mass” unless otherwise specified.

In the present specification, DMF means N,N-dimethylformamide, and MEHQ means 4-methoxyphenol.

### <Synthesis of Electron-Transporting Compound>

Next, synthetic methods of a compound 1 to a compound 5 and comparative compounds 1 and 2 as electron-transporting compounds will be explained.

### [Synthesis of Compound 1]

The synthesis scheme of a compound 1 is shown below.

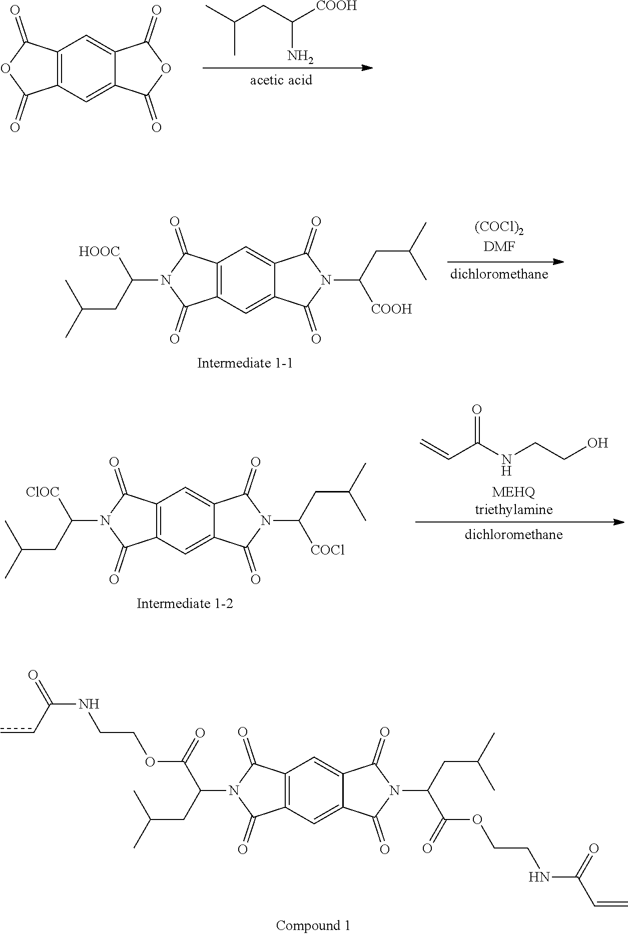

The synthesis procedures of the compound 1 are shown below.

### (Synthesis of Intermediate 1-1)

In nitrogen atmosphere, 250 mL of acetic acid was added to a mixture of benzene-1,2,4,5-tetracarboxylic anhydride (19.3 g, 88.4 mmol) and L-leucine (23.2 g, 176.9 mmol), and the mixture was stirred at room temperature for 12 hours and stirred at 130° C. for 12 hours. After cooling to room temperature, the mixture was poured into 400 mL of ice water, and the solid was filtered and washed with water. After drying, an intermediate 1-1 (an amount of 38.7 g, a yield of 98%) was obtained.

### (Synthesis of Intermediate 1-2)

In nitrogen atmosphere, 200 mL of dehydrated dichloromethane and 1 mL of dehydrated dimethylformamide were added to the intermediate 1-1 (23.0 g, 51.8 mmol), and the mixture was ice-cooled. Oxalyl chloride (26.6 mL, 310.5 mmol) was added dropwise, and the mixture was stirred under ice-cooling for two hours and stirred at room temperature for 12 hours. After evaporating the solvent at reduced pressure, the solid was filtered/washed with hexane. After drying, an intermediate 1-2 (an amount of 23.5 g, a yield of 94%) was obtained.

### (Synthesis of Compound 1)

In nitrogen atmosphere, after adding 4-methoxyphenol (0.01 g) to N-(2-hydroxyethyl)acrylamide (17.9 g, 51.7 mmol), 200 mL of dehydrated dichloromethane and triethylamine (43 mL, 310.2 mmol) were further added, and the mixture was ice-cooled. The intermediate 1-2 (23.5 g, 48.8 mmol) which was dissolved in 100 mL of dehydrated dichloromethane was added dropwise, and the mixture was stirred under ice-cooling for two hours and stirred at room temperature for two hours. After evaporating the solvent at reduced pressure, the residue was subjected to silica gel column chromatography, and the compound 1 (an amount of 14.2 g, a yield of 46%) was obtained.

[Synthesis of Compound 2]

The synthesis scheme of a compound 2 is shown below. Here, the compound 2 is a mixture of a compound 2-a, a compound 2-b and a compound 2-c.

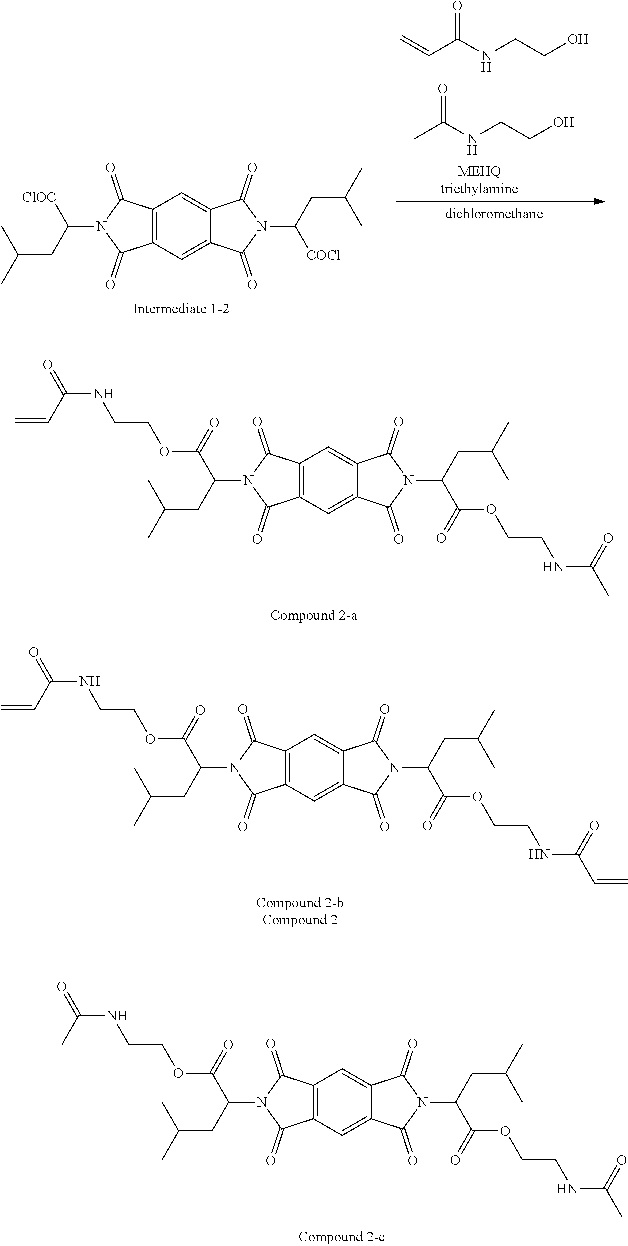

The synthesis procedures of the compound 2 are shown below.

### (Synthesis of Compound 2)

In nitrogen atmosphere, 200 mL of dehydrated dichloromethane and triethylamine (29 mL, 208.2 mmol) were added to a mixture of N-(2-hydroxyethyl)acrylamide (5.99 g, 52.0 mmol), 2-acetamidoethanol (5.37 g, 52.0 mmol) and 4-methoxyphenol (0.03 g), and the mixture was ice-cooled. A solution obtained by dissolving the intermediate 1-2 (16.7 g, 34.7 mmol) obtained by the procedures described above in 100 mL of dehydrated dichloromethane was added dropwise, and the mixture was stirred under ice-cooling for two hours and stirred at room temperature for two hours. After evaporating the solvent at reduced pressure, the residue was subjected to silica gel column chromatography, and the compound 2 (an amount of 3.4 g, a yield of 16%) was obtained.

### [Synthesis of Compound 3]

The synthesis scheme of a compound 3 is shown below.

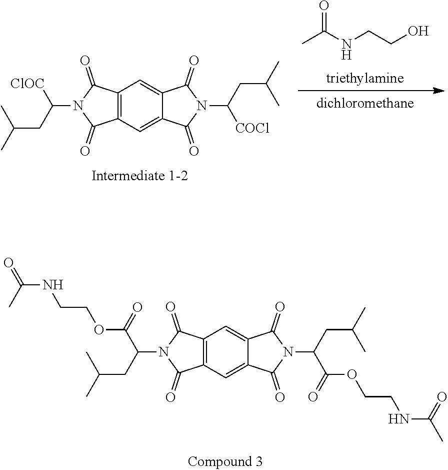

The synthesis procedures of the compound 3 are shown below.

### (Synthesis of Compound 3)

In nitrogen atmosphere, 150 mL of dehydrated dichloromethane and triethylamine (10.3 mL, 74.4 mmol) were added to 2-acetamidoethanol (3.9 g, 37.4 mmol), and the mixture was ice-cooled. A solution obtained by dissolving the intermediate 1-2 (6.0 g, 12.4 mmol) obtained by the procedures described above in 50 mL of dehydrated dichloromethane was added dropwise, and the mixture was stirred under ice-cooling for two hours and stirred at room temperature for two hours. After evaporating the solvent at reduced pressure, the residue was subjected to silica gel column chromatography, and a compound 3 (an amount of 3.6 g, a yield of 47%) was obtained.

### [Synthesis of Compound 4]

The synthesis scheme of a compound 4 is shown below.

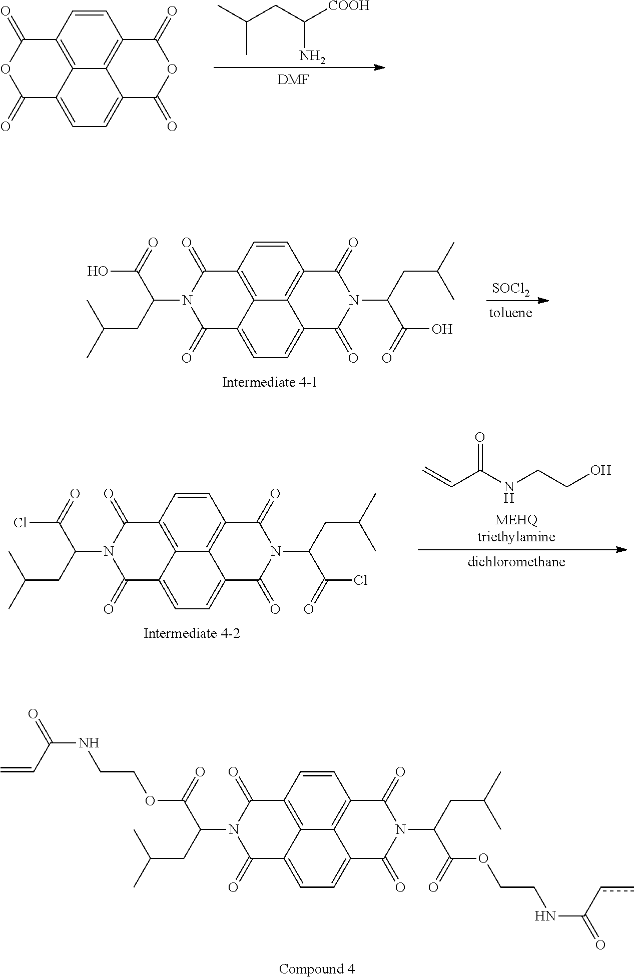

The synthesis procedures of the compound 4 are shown below.

### (Synthesis of Intermediate 4-1)

In nitrogen atmosphere, 100 mL of N,N-dimethylformamide was added to a mixture of naphthalene-1,4,5,8-tetracarboxylic dianhydride (4.13 g, 15.4 mmol) and L-leucine (6.06 g, 46.2 mmol), and the mixture was stirred at 85° C. for 8.5 hours. After cooling to room temperature, the mixture was poured into 200 mL of ice water, and the reaction solution was made acidic by adding 1 N hydrochloric acid. Extraction with ethyl acetate was conducted, and the organic layer was washed with water and then dried over magnesium sulfate. The solid was filtered. The solvent of the filtrate was evaporated at reduced pressure, and after drying the residue, an intermediate 4-1 (an amount of 6.9 g, a yield of 91%) was obtained.

### (Synthesis of Intermediate 4-2)

In nitrogen atmosphere, 80 mL of toluene was added to the intermediate 4-1 (4.0 g, 8.09 mmol), and the mixture was ice-cooled. Thionyl chloride (7.0 mL, 97.1 mmol) was added dropwise, and the mixture was stirred at 85° C. for four hours. After cooling to room temperature, the solvent was evaporated at reduced pressure, and then the solid was filtered/washed with hexane. After drying, an intermediate 4-2 (an amount of 4.3 g, a yield of 99%) was obtained.

### (Synthesis of Compound 4)

In nitrogen atmosphere, 30 mL of dehydrated dichloromethane and triethylamine (6.7 mL, 48.5 mmol) were added to a mixture of N-(2-hydroxyethyl)acrylamide (2.79 g, 24.3 mmol) and 4-methoxyphenol (0.02 g), and the mixture was ice-cooled. The intermediate 4-2 (4.3 g, 8.09 mmol) which was dissolved in 50 mL of dehydrated dichloromethane was added dropwise, and the mixture was stirred under ice-cooling for an hour and stirred at room temperature for two hours. After evaporating the solvent at reduced pressure, the residue was subjected to silica gel column chromatography, and the compound 4 (an amount of 1.6 g, a yield of 29%) was obtained.

### [Synthesis of Compound 5]

The synthesis scheme of a compound 5 is shown below.

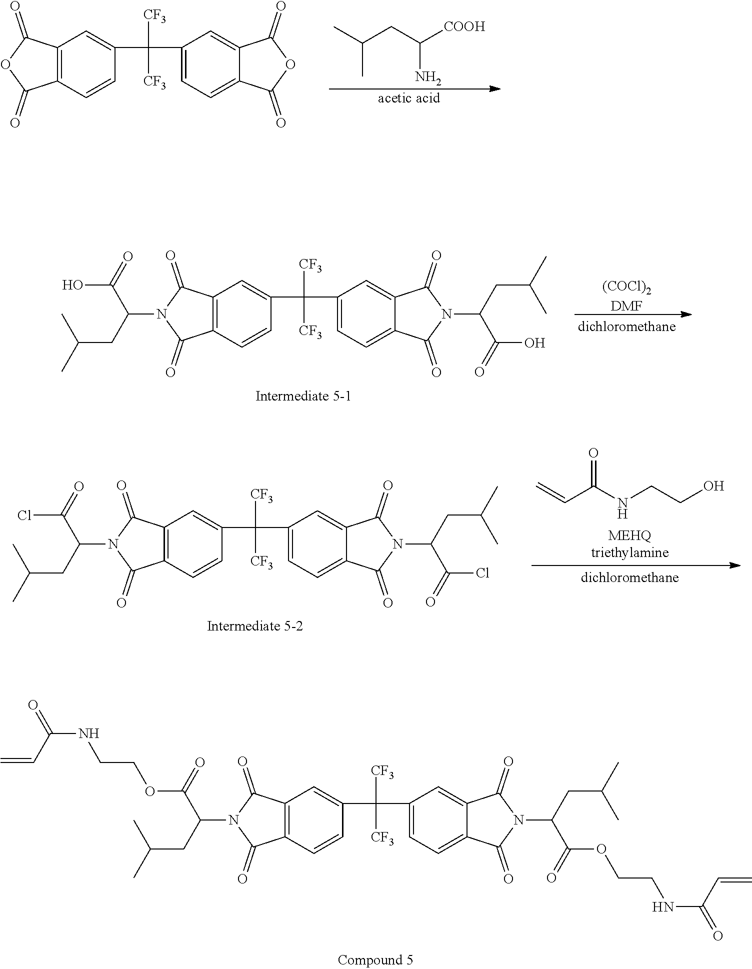

The synthesis procedures of the compound 5 are shown below.

### (Synthesis of Intermediate 5-1)

In nitrogen atmosphere, 250 mL of acetic acid was added to a mixture of 4,4′-(hexafluoroisopropylidene)diphthalic anhydride (20.0 g, 45.0 mmol) and L-leucine (11.8 g, 90.0 mmol), and the mixture was stirred at room temperature for 12 hours and stirred at 130° C. for nine hours. After cooling to room temperature, the mixture was poured into 400 mL of ice water, and the solid was filtered and washed with water. After drying, an intermediate 5-1 (an amount of 30.1 g, a yield of 99%) was obtained.

### (Synthesis of Intermediate 5-2)

In nitrogen atmosphere, 100 mL of dehydrated dichloromethane and 1 mL of dehydrated dimethylformamide were added to the intermediate 5-1 (12.6 g, 18.8 mmol), and the mixture was ice-cooled. Oxalyl chloride (6.4 mL, 75.2 mmol) was added dropwise, and the mixture was stirred under ice-cooling for two hours and stirred at room temperature for 12 hours. After evaporating the solvent at reduced pressure, the solid was filtered/washed with hexane. After drying, an intermediate 5-2 (an amount of 13.0 g, a yield of 98%) was obtained.

### (Synthesis of Compound 5)

In nitrogen atmosphere, 100 mL of dehydrated dichloromethane and triethylamine (15.3 mL, 110.4 mmol) were added to a mixture of N-(2-hydroxyethyl)acrylamide (6.35 g, 55.1 mmol) and 4-methoxyphenol (0.03 g), and the mixture was ice-cooled. The intermediate 5-2 (13.0 g, 18.4 mmol) which was dissolved in 30 mL of dehydrated dichloromethane was added dropwise, and the mixture was stirred under ice-cooling for an hour and stirred at room temperature for two hours. After evaporating the solvent at reduced pressure, the residue was subjected to silica gel column chromatography, and the compound 5 (an amount of 6.9 g, a yield of 43%) was obtained.

### [Synthesis of Compound 6]

The synthesis scheme of a compound 6 is shown below.

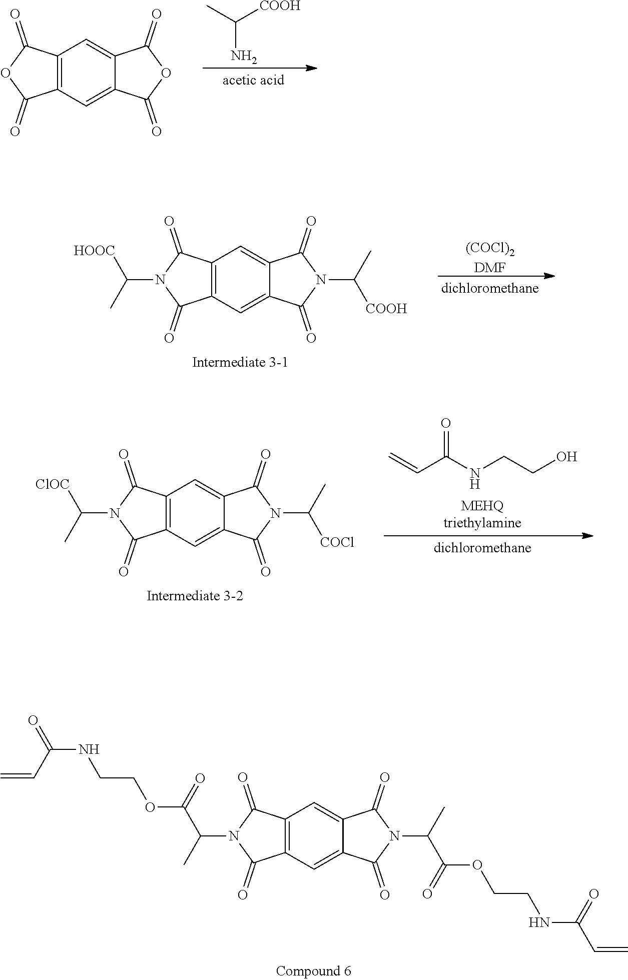

The synthesis procedures of the compound 6 are shown below.

### (Synthesis of Intermediate 3-1)

In nitrogen atmosphere, 100 mL of acetic acid was added to a mixture of benzene-1,2,4,5-tetracarboxylic anhydride (6.29 g, 28.8 mmol) and L-alanine (5.65 g, 63.4 mmol), and the mixture was stirred at room temperature for 12 hours and stirred at 130° C. for 12 hours. After cooling to room temperature, the mixture was poured into 400 mL of ice water, and the solid was filtered and washed with water. After drying, an intermediate 3-1 (an amount of 6.8 g, a yield of 65%) was obtained.

### (Synthesis of Intermediate 3-2)

In nitrogen atmosphere, 150 mL of dehydrated dichloromethane and 1 mL of dehydrated dimethylformamide were added to the intermediate 3-1 (6.6 g, 18.3 mmol), and the mixture was ice-cooled. Oxalyl chloride (9.4 mL, 109.9 mmol) was added dropwise, and the mixture was stirred under ice-cooling for two hours and stirred at room temperature for 12 hours. After evaporating the solvent at reduced pressure, the solid was filtered/washed with hexane. After drying, an intermediate 3-2 (an amount of 7.1 g, a yield of 98%) was obtained.

### (Synthesis of Compound 6)

In nitrogen atmosphere, 100 mL of dehydrated dichloromethane and triethylamine (15 mL, 108.6 mmol) were added to a mixture of N-(2-hydroxyethyl)acrylamide (6.3 g, 54.4 mmol) and 4-methoxyphenol (0.03 g), and the mixture was ice-cooled. The intermediate 3-2 (7.2 g, 18.1 mmol) which was dissolved in 30 mL of dehydrated dichloromethane was added dropwise, and the mixture was stirred under ice-cooling for two hours and stirred at room temperature for two hours. After evaporating the solvent at reduced pressure, the residue was subjected to silica gel column chromatography, and the compound 6 (an amount of 3.8 g, a yield of 38%) was obtained.

### [Synthesis of Comparative Compound 1]

The synthesis scheme of a comparative compound 1 is shown below.

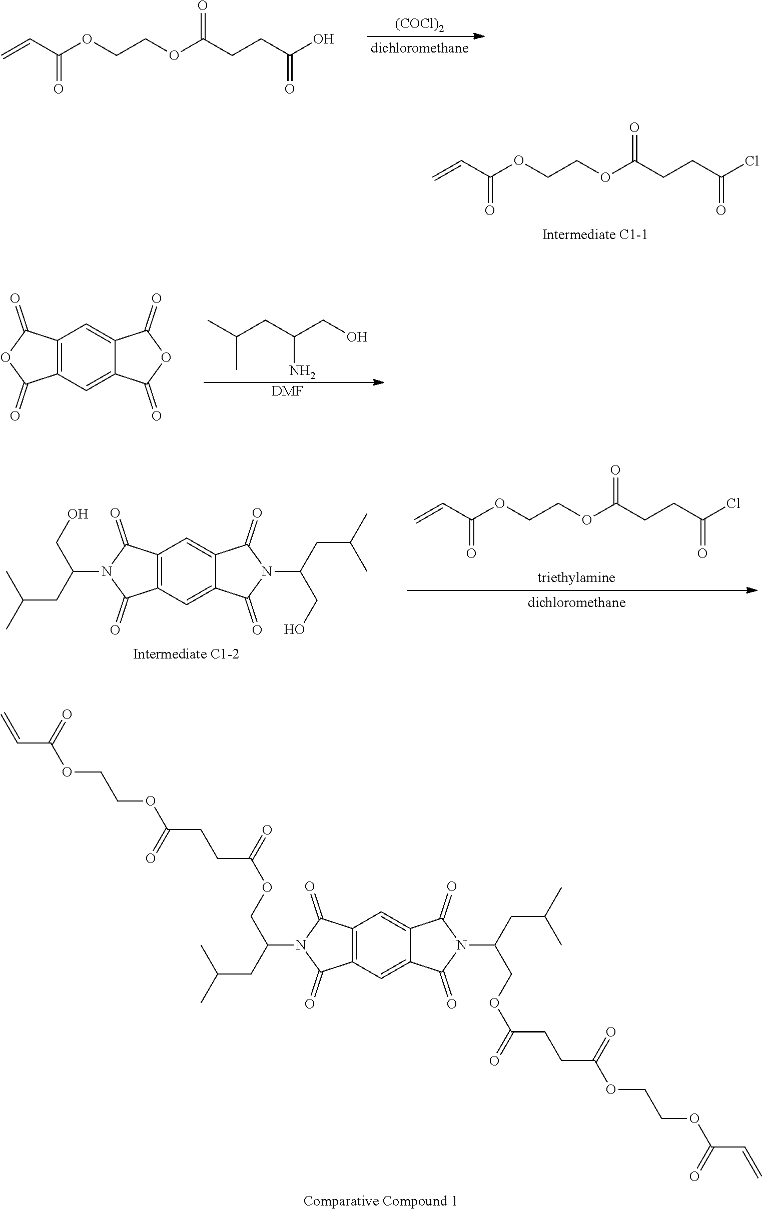

The synthesis procedures of the comparative compound 1 are shown below.

### (Synthesis of Intermediate C1-1)

In nitrogen atmosphere, 200 mL of dehydrated dichloromethane was added to mono(2-acryloyloxyethyl) succinate (21.7 g, 100.4 mmol), and the mixture was ice-cooled. Oxalyl chloride (11.2 mL, 130.5 mmol) was added dropwise, and the mixture was stirred under ice-cooling for an hour and stirred at room temperature for 12 hours. The solvent in the reaction solution was evaporated at reduced pressure, and the residue was dried. Thus, an intermediate C1-1 (an amount of 23 g, a yield of 97%) was obtained.

### (Synthesis of Intermediate C1-2)

In nitrogen atmosphere, 100 mL of N,N-dimethylformamide was added to a mixture of benzene-1,2,4,5-tetracarboxylic anhydride (5.0 g, 22.9 mmol) and L-(+)-leucinol (5.9 mL, 45.8 mmol), and the mixture was stirred at 120° C. for nine hours. After cooling to room temperature, the mixture was poured into 200 mL of ice water, and the solution was made acidic by adding 1 N hydrochloric acid. The solid was filtered and washed with water. After drying, an intermediate C1-2 (an amount of 8.2 g, a yield of 86%) was obtained.

### (Synthesis of Comparative Compound 1)

In nitrogen atmosphere, 100 mL of dehydrated dichloromethane and triethylamine (7.0 mL, 50.4 mmol) were added to the intermediate C1-2 (5.2 g, 12.6 mmol), and the mixture was ice-cooled. The intermediate C1-1 (6.5 g, 27.7 mmol) which was dissolved in 100 mL of dehydrated dichloromethane was added dropwise, and the mixture was stirred under ice-cooling for an hour and stirred at room temperature for an hour. The reaction solution was poured into 100 mL of water, and extraction with dichloromethane was conducted. The organic layer was washed with water and then dried over magnesium sulfate. The solid was filtered, and the solvent of the filtrate was evaporated at reduced pressure. The residue was subjected to silica gel column chromatography, and the comparative compound 1 (an amount of 7.6 g, a yield of 75%) was obtained.

### [Synthesis of Comparative Compound 2]

The synthesis scheme of a comparative compound 2 is shown below.

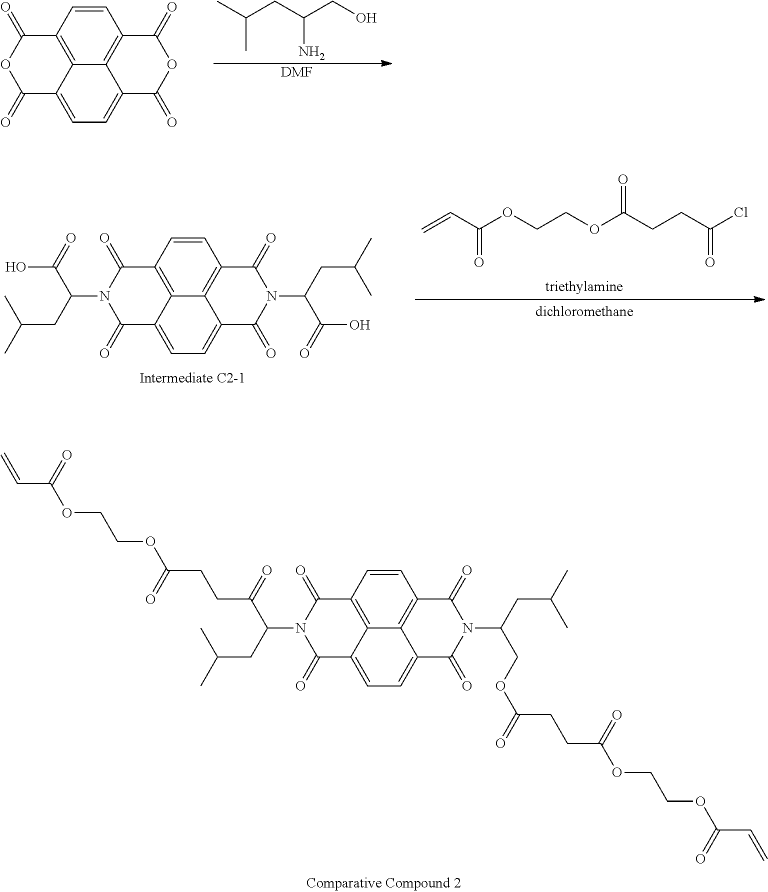

The synthesis procedures of the comparative compound 2 are shown below.

### (Synthesis of Intermediate C2-1)

In nitrogen atmosphere, 150 mL of N,N-dimethylformamide was added to a mixture of naphthalene-1,4,5,8-tetracarboxylic dianhydride (4.5 g, 16.9 mmol) and L-(+)-leucinol (4.4 mL, 33.7 mmol), and the mixture was stirred at 150° C. for six hours. After cooling to room temperature, the mixture was poured into 200 mL of ice water, and the solution was made acidic by adding 1 N hydrochloric acid. Extraction with ethyl acetate was conducted, and the organic layer was washed with water and then dried over magnesium sulfate. The solid was filtered. The solvent of the filtrate was evaporated at reduced pressure, and after drying the residue, an intermediate C2-1 (an amount of 7.8 g, a yield of 99%) was obtained.

### (Synthesis of Comparative Compound 2)

In nitrogen atmosphere, 50 mL of dehydrated dichloromethane and triethylamine (5.1 mL, 36.8 mmol) were added to the intermediate C2-1 (4.3 g, 9.21 mmol), and the mixture was ice-cooled. The intermediate C1-1 (4.8 g, 20.3 mmol) which was dissolved in 50 mL of dehydrated dichloromethane was added dropwise, and the mixture was stirred under ice-cooling for an hour and stirred at room temperature for an hour. The reaction solution was poured into 100 mL of water, and extraction with dichloromethane was conducted. The organic layer was washed with water and then dried over magnesium sulfate. The solid was filtered, and the solvent of the filtrate was evaporated at reduced pressure. The residue was subjected to silica gel column chromatography, and the comparative compound 2 (an amount of 3.5 g, a yield of 45%) was obtained.

### <<Evaluation of Solubility in Organic Solvent>>

### Examples 1-1 to 1-5 and Comparative Examples 1-1 and 1-2

To 100 parts by mass of methanol at room temperature (25° C.), eight parts by mass of the compounds 1 to 5 or the comparative compounds 1 and 2 were added, and without heating or cooling, the mixtures were stirred with a rotor for 10 minutes. Then, the dissolution states were observed visually, and the solubility was evaluated by the following criteria.

When undissolved compound was observed, the dissolution state was observed visually again after heating in a water bath at a constant temperature (40° C.) for 10 minutes as described below, and the solubility was evaluated. The evaluation results are shown in Table 1.

A (very good): The compound dissolved completely at room temperature.

B (good): Undissolved compound was observed at room temperature, and the compound dissolved completely after heating at 40° C. for 10 minutes.

C (poor): Undissolved compound was observed at room temperature and after heating at 40° C. for 10 minutes.

TABLE 1

| | Electron-Transporting | |
|---|---|---|
| | Compound | Solubility |
| Example 1-1 | Compound 1 | B |
| Example 1-2 | Compound 2 | A |
| Example 1-3 | Compound 3 | A |
| Example 1-4 | Compound 4 | B |
| Example 1-5 | Compound 5 | A |
| Comparative Example 1-1 | Comparative Compound 1 | C |
| Comparative Example 1-2 | Comparative Compound 2 | C |

### <Discussion>

From the evaluation of the solubility, the results of the tests which were conducted so far by the present inventors and the like, it was found that the novel compound represented by the following formula (1) has an electron-transporting structure and an amide bond structure and dissolves sufficiently in an organic solvent. Moreover, it was found that the novel compound represented by the following formula (1) has electron-transporting property and is useful as an electron-transporting compound of an electrophotographic photoreceptor.

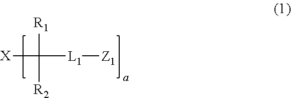

In the formula (1), X represents an electron-transporting skeleton. R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents, and at least one of R₁ and R₂ is an alkyl group which has two or more carbon atoms and which may have one or more substituents. L₁ represents a divalent group. Z₁ represents an amide group (—NHCO—R′), an acrylamide group, a methacrylamide group, an acryloyl group or a methacryloyl group. Z₁ represents an amide group, an acrylamide group or a methacrylamide group when a is 1, and at least one thereof represents an amide group, an acrylamide group or a methacrylamide group when a is an integer of 2 or more. R′ represents a hydrogen atom, an alkyl group which may have one or more substituents, an aralkyl group which may have one or more substituents or an aromatic group which may have one or more substituents. a represents an integer of 1 or more. When a is an integer of 2 or more, R₁, R₂, L₁ and Z₁ in each of the repeating structures may be the same or different from each other.

### <<Production of Electrophotographic Photoreceptor>>

### (Production of Coating Liquid P1 for Forming Undercoat Layer)

Twenty parts of D-form titanyl phthalocyanine, which exhibits a clear peak at a diffraction angle of 2θ=27.3°±0.2° in powder X-ray diffraction using CuKα rays, and 280 parts of 1,2-dimethoxyethane were mixed, and the mixture was ground with a sand grind mill for two hours to conduct atomization dispersion treatment. Furthermore, 400 parts of 1,2-dimethoxyethane solution containing 2.5 mass % polyvinyl butyral (manufactured by Denki Kagaku Kogyo Kabushiki Kaisha, product name “Denka Butyral” #6000C) and 170 parts of 1,2-dimethoxyethane were added thereto and mixed, and a coating liquid P1 for forming an undercoat layer having a solid concentration of 3.4 mass % was produced.

### (Production of Coating Liquid Q1 for Forming Single-Layer Photosensitive Layer)

To 793.35 parts of a mixed solvent of tetrahydrofuran (appropriately abbreviated to as THF below) and toluene (appropriately abbreviated to as TL below) (80 mass % THF and 20 mass % TL), 2.6 parts of D-form titanyl phthalocyanine, which exhibits a clear peak at a diffraction angle of 2θ=27.3°±0.2° in powder X-ray diffraction using CuKα rays, 1.3 parts of a perylene pigment 1 having the structure below, 0.5 parts of a polyvinyl butyral resin, 100 parts of the hole transport material below (HTM48, molecular weight of 748), 60 parts of the electron transport material below (ET-2, molecular weight of 424.2), 100 parts of a polycarbonate resin having a biphenyl structure and 0.05 parts of silicone oil (manufactured by Shin-Etsu Chemical Co., Ltd.: product name KF-96) as a leveling agent were added and mixed, and a coating liquid Q1 for forming a single-layer photosensitive layer having a solid concentration of 25 mass % was produced.

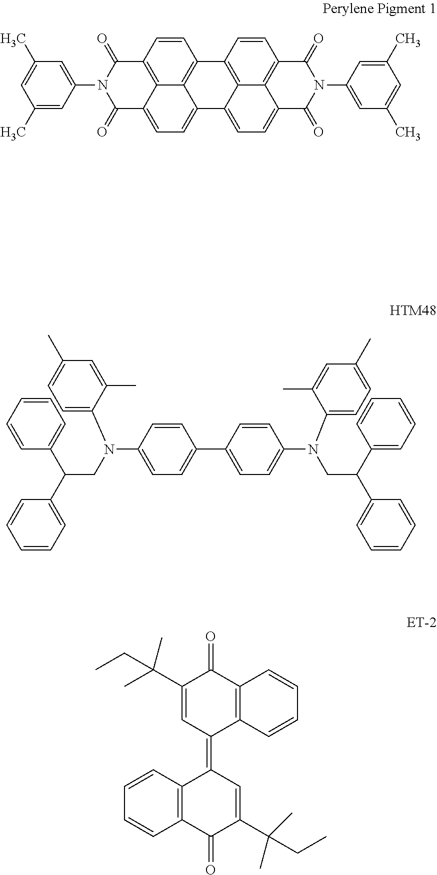

### (Production of Coating Liquid S1 for Forming Protective Layer)

A mixed solvent of methanol/1-propanol, benzophenone and Omnirad TPO H (2,4,6-trimethylbenzoyl-diphenylphosphine oxide) as polymerization initiators and the compound 1 as an electron-transporting compound were mixed, and thus a coating liquid S1 for forming a protective layer (solid concentration of 8.0 mass %) with electron-transporting compound/benzophenone/Omnirad TPO H=(mass ratio) 100/1/2 and a solvent composition of methanol/1-propanol=(mass ratio) 8/2 was obtained.

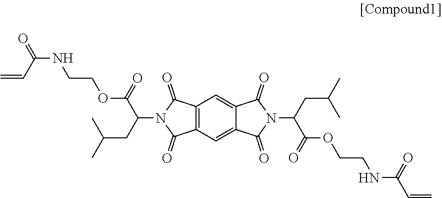 [SMILES: C=CC(=O)NCCOC(=O)C(CC(C)C)n1c(=O)c2cc3c(=O)n(C(CC(C)C)C(=O)OCCNC(=O)C=C)c(=O)c3cc2c1=O]

### (Production of Coating Liquid S2 for Forming Protective Layer)

A curable compound (polyester acrylate: manufactured by Toagosei Co., Ltd., product name “Aronix M-9050”) which was dissolved in a mixed solvent of methanol/1-propanol in advance, benzophenone and Omnirad TPO H (2,4,6-trimethylbenzoyl-diphenylphosphine oxide) as polymerization initiators and the compound 1 as an electron-transporting compound were mixed, and thus a coating liquid S2 for forming a protective layer (solid concentration of 8.0 mass %) with electron-transporting compound/M-9050/benzophenone/Omnirad TPO H=(mass ratio) 100/50/1/2 and a solvent composition of methanol/1-propanol=(mass ratio) 8/2 was obtained.

### (Production of Coating Liquids S3 to S15 for Forming Protective Layer)

Coating liquids S3 to S15 for forming a protective layer were obtained in the same manner as the coating liquid S1 for forming a protective layer except that the kind of the electron-transporting compound and the amount of the curable compound (M-9050) were changed as shown in Table 2.

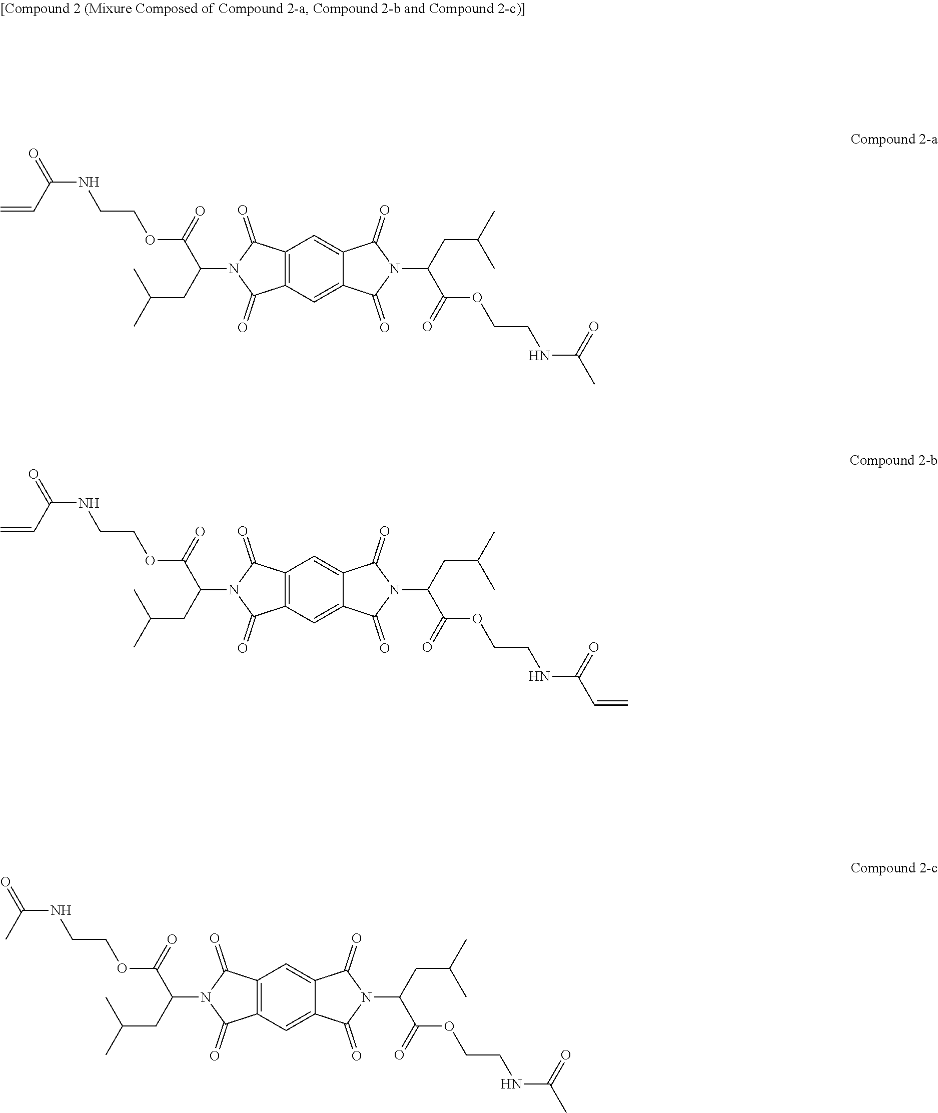 [SMILES: C=CC(=O)NCCOC(=O)C(CC(C)C)n1c(=O)c2cc3c(=O)n(C(CC(C)C)C(=O)OCCNC(C)=O)c(=O)c3cc2c1=O ; C=CC(=O)NCCOC(=O)C(CC(C)C)n1c(=O)c2cc3c(=O)n(C(CC(C)C)C(=O)OCCNC(=O)C=C)c(=O)c3cc2c1=O ; CC(=O)NCCOC(=O)C(CC(C)C)n1c(=O)c2cc3c(=O)n(C(CC(C)C)C(=O)OCCNC(C)=O)c(=O)c3cc2c1=O]

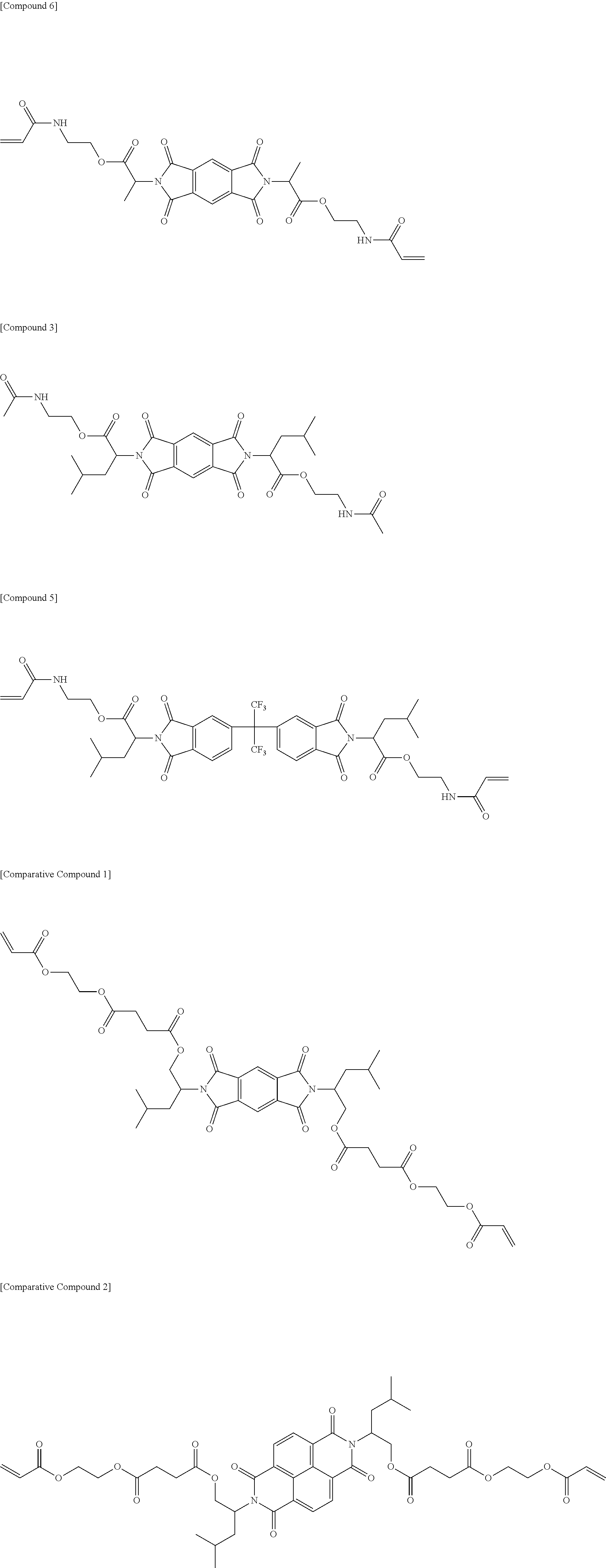

### <Production of Single-Layer Photoreceptor>

Single-layer photoreceptors were produced by the procedures below.

### Example 2-1

An aluminum cylinder of 30 mmφ and a length of 244 mm having a surface subjected to cutting treatment was coated with the coating liquid P1 for forming an undercoat layer by dip coating, and thus an undercoat layer was provided in such a manner that the thickness after drying became 0.3 μm. The coating liquid Q1 for forming a single-layer photosensitive layer was applied on the undercoat layer by dip coating and dried at 125° C. for 24 minutes, and thus a single-layer photosensitive layer was provided in such a manner that the thickness after drying became 32 μm. The coating liquid S1 for forming a protective layer was applied on the single-layer photosensitive layer by ring coating, and soon after the application, LED light of 365 nm was applied at an intensity of 0.9 W/cm² for two minutes while the photoreceptor was rotated at 60 rpm in nitrogen atmosphere to provide a protective layer in such a manner that the thickness after curing became 1 μm. Thus, a photoreceptor A1 was produced.

### Examples 2-2 to 2-13 and Comparative Examples 2-1 and 2-2

Photoreceptors A2 to A15 were produced in the same manner as the photoreceptor A1 except that the coating liquid S1 for forming a protective layer was changed to the coating liquids S2 to S15 for forming a protective layer.

### <Electrical Property: Evaluation of Residual Potential>

The photoreceptors A1 to A15 obtained in the Examples and the Comparative Examples were attached to a device for evaluating electrophotographic properties produced according to the measurement standards of the Society of Electrophotography (described in Sequel to Basics and Application of Electrophotographic Technology, edited by the Society of Electrophotography, Corona Publishing Co., Ltd., pages 404 to 405), and the electrical properties after charging, exposure, potential measurement and charge elimination cycles were measured as follows.

First, the grid voltage was adjusted, and the photoreceptor was charged to an initial surface potential (V0) of +700 V. Next, 1.3 μJ/cm² of exposure light was applied, and the residual potential (VL) 60 milliseconds after the application was measured. Here, as the exposure light, monochromatic light of 780 nm from the light of a halogen lamp through an interference filter was used. The measurement environment was at a temperature of 25° C. and at a relative humidity of 50% (N/N environment).

The obtained residual potential (VL) was evaluated by the following criteria based on the absolute value. The results are shown in Table 2. As the absolute value of the residual potential (VL) is smaller, the potential lowers because the charge is sufficiently transported, which is considered a good result.

In the present invention, the evaluation result of “C” or better was considered to be “acceptable”.

A (excellent): The absolute value of the residual potential (VL) is 149 V or less.

B (very good): The absolute value of the residual potential (VL) is 150 V or more and 199 V or less.

C (good): The absolute value of the residual potential (VL) is 200 V or more and 259 V or less.

D (poor): The absolute value of the residual potential (VL) is 260 V or more.

### <Electrical Property: Evaluation of Potential Retention Rate>

The photoreceptors A1 to A15 obtained in the Examples and the Comparative Examples were attached to a device for evaluating electrophotographic properties produced according to the measurement standards of the Society of Electrophotography (described in Sequel to Basics and Application of Electrophotographic Technology, edited by the Society of Electrophotography, Corona Publishing Co., Ltd., pages 404 to 405), and the electrical properties after charging, exposure, potential measurement and charge elimination cycles were measured as follows.

To evaluate the electrical property, the potential retention rate (DDR) after being charged to +700 V and leaving for five seconds was measured (%). The measurement environment was at a temperature of 25° C. and at a relative humidity of 50% (N/N environment).

The obtained potential retention rate (DDR) was evaluated by the following criteria based on the value. The results are shown in Table 2. The potential retention rate (DDR) indicates the retention rate (%) of the surface potential after the photoreceptor with a charged surface is left for a certain period. When the retention rate (%) of the surface potential is large, the potential is maintained after time passes, and the electrostatic property is excellent, which is considered a good result. In the present invention, the evaluation result of “C” or better was considered “acceptable”.

A (excellent): The potential retention rate (DDR) is 91% or more.

B (very good): The potential retention rate (DDR) is 86% or more and 90% or less.

C (good): The potential retention rate (DDR) is 81% or more and 85% or less.

D (poor): The potential retention rate (DDR) is 80% or less.

TABLE 2

| | | Protective Layer | | | Electrical Property | |
|---|---|---|---|---|---|---|
| | | Coating Liquid | Electron- | | | Potential |
| | | for Forming | Transporting | Curable | Residual | Retention |
| | | Protective | Compound | Compound | Potential | Rate |
| | Photoreceptor | Layer | (parts by mass) | (parts by mass) | VL | DDR |
| Example 2-1 | A1 | S1 | Compound 1 (100) | M-9050 (0) | A | A |
| Example 2-2 | A2 | S2 | Compound 1 (100) | M-9050 (50) | B | A |
| Example 2-3 | A3 | S3 | Compound 1 (100) | M-9050 (100) | C | A |
| Example 2-4 | A4 | S4 | Compound 2 (100) | M-9050 (0) | A | A |
| Example 2-5 | A5 | S5 | Compound 2 (100) | M-9050 (50) | A | A |
| Example 2-6 | A6 | S6 | Compound 2 (100) | M-9050 (100) | B | A |
| Example 2-7 | A7 | S7 | Compound 6 (100) | M-9050 (0) | A | B |
| Example 2-8 | A8 | S8 | Compound 6 (100) | M-9050 (50) | A | B |
| Example 2-9 | A9 | S9 | Compound 6 (100) | M-9050 (100) | A | B |
| Example 2-10 | A10 | S10 | Compound 3 (100) | M-9050 (100) | A | C |
| Example 2-11 | A11 | S11 | Compound 5 (100) | M-9050 (0) | C | A |
| Example 2-12 | A12 | S12 | Compound 5 (100) | M-9050 (50) | C | A |
| Example 2-13 | A13 | S13 | Compound 5 (100) | M-9050 (100) | C | A |
| Comparative | A14 | S14 | Comparative | M-9050 (100) | D | C |
| Example 2-1 | | | Compound 1 (100) | | | |
| Comparative | A15 | S15 | Comparative | M-9050 (100) | D | C |
| Example 2-2 | | | Compound 2 (100) | | | |

### <Measurement of Martens Hardness and Elastic Deformation Ratio>

The Martens hardnesses and the elastic deformation ratios of the photoreceptors A3, A6 and A10 obtained in the Examples were measured in an environment at a temperature of 25° C. at a relative humidity of 50% using a micro-hardness meter (manufactured by Fischer: FISCHERSCOPE HM2000) from the surface side of the photoreceptor under the following measurement conditions. The Martens hardnesses and the elastic deformation ratios of the samples are shown in Table 3.

### (Measurement Conditions of Martens Hardness and Elastic Deformation Ratio)

Indenter: Vickers pyramid diamond indenter having a facing angle of 136°
Maximum indentation load: 0.2 mN
Loading time: 10 seconds
Unloading time: 10 seconds

The Martens hardness was determined by the equation below.

Martens Hardness (N/mm²)=Maximum Indentation Load/Indent Area at Maximum Indentation Load

The elastic deformation ratio is a value defined by the equation below and is the proportion of the work performed by the film due to elasticity during unloading in the total work required for indentation.

$$Elastic Deformation Ratio ( \% ) = ( We / Wt ) \times 100$$

In the above equation, the total work Wt (nJ) indicates the area surrounded by A-B-D-A in FIG. 2, and the elastic deformation work We (nJ) indicates the area surrounded by C-B-D-C. As the elastic deformation ratio is larger, the deformation due to the load does not remain easily, and the elastic deformation ratio of 100 means that the deformation does not remain.

TABLE 3

| | | Protective Layer | | | | |
|---|---|---|---|---|---|---|
| | | Coating | | | | |
| | | Liquid for | Electron- | | | Elastic |
| | | Forming | Transporting | Curable | Martens | Deformation |
| | | Protective | Compound | Compound | Hardness | Ratio |
| | Photoreceptor | Layer | (parts by mass) | (parts by mass) | (N/mm²) | (%) |
| Example 2-3 | A3 | S3 | Compound 1 (100) | M-9050 (100) | 209.6 | 30.1 |
| Example 2-6 | A6 | S6 | Compound 2 (100) | M-9050 (100) | 233.3 | 29.1 |
| Example 2-10 | A10 | S10 | Compound 3 (100) | M-9050 (100) | 76.5 | 14.2 |

### DISCUSSION

From the results in Table 2, excellent effects for both the residual potential property and the potential retention rate were observed in all of Examples 2-1 to 2-13.

This shows that an electrophotographic photoreceptor having at least a photosensitive layer and a protective layer in this order on a conductive support in which the protective layer contains an electron-transporting compound represented by the following formula (1) can achieve electrical properties, in particular both residual potential property and potential retention rate, even when a compound having an electron-transporting structure is contained in the protective layer.

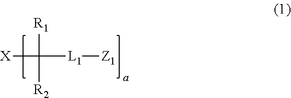

In the formula (1), X represents an electron-transporting skeleton. R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents. L₁ represents a divalent group. Z₁ represents an amide group (—NHCO—R′), an acrylamide group, a methacrylamide group, an acryloyl group or a methacryloyl group. Z₁ represents an amide group, an acrylamide group or a methacrylamide group when a is 1, and at least one thereof represents an amide group, an acrylamide group or a methacrylamide group when a is an integer of 2 or more. R′ represents a hydrogen atom, an alkyl group which may have one or more substituents, an aralkyl group which may have one or more substituents or an aromatic group which may have one or more substituents. a represents an integer of 1 or more. When a is an integer of 2 or more, R₁, R₂, L₁ and Z₁ in each of the repeating structures may be the same or different from each other.

Moreover, it was found that, when Example 2-1 and Example 2-7, Example 2-2 and Example 2-8 and Example 2-3 and Example 2-9 were each compared, the dark decoy properties of Examples 2-1 to 2-3 (the compound 1) were superior to those of Examples 2-7 to 2-9 (the compound 3). This shows that a further excellent effect in terms of dark decoy can be obtained when at least one of R₁ and R₂ in the formula (1) is an alkyl group which has two or more carbon atoms and which may have one or more substituents.

Moreover, from the results in Table 3, it was found that a superior effect in terms of the Martens hardness and the elastic deformation ratio could be obtained in Examples 2-3 and 2-6 compared to Example 2-10.

This shows that the Martens hardness and the elastic deformation ratio are further preferable when at least one or more of Z₁s in the formula (1) are an acrylamide group or a methacrylamide group.

This is believed to be because, when the amide bond is contained in the structure of the compound as an acrylamide group or a methacrylamide group, as in Examples 2-3 and 2-6, the amide bond can also play the role of a chain-polymerizable functional group and can be cross-linked with the curable compound in the protective layer.

### REFERENCE SIGNS LIST

1: Photoreceptor (electrophotographic photoreceptor)
2: Charging device (charging roller; charging member)
3: Exposure device (exposure member)
4: Developing device (developing member)
5: Transfer device
6: Cleaning device
7: Fixing device
41: Developer tank
42: Agitator
43: Supply roller
44: Developing roller
45: Regulating member
71: Upper fixing member (pressure roller)
72: Lower fixing member (fixing roller)
73: Heating device
T: Toner
P: Recording sheet (paper, medium)

1. An electrophotographic photoreceptor comprising a photosensitive layer and a protective layer in this order on a conductive support,
wherein the protective layer comprises an electron-transporting compound represented by the following formula (1): 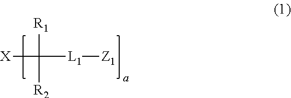
in the formula (1),
X represents an electron-transporting skeleton,
R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents,
L₁ represents a divalent group,
Z₁ represents an amide group (—NHCO—R′), an acrylamide group, a methacrylamide group, an acryloyl group or a methacryloyl group, wherein when a is 1, Z₁ represents an amide group, an acrylamide group or a methacrylamide group, when a is an integer of 2 or more, at least one Z₁ represents an amide group, an acrylamide group or a methacrylamide group, and R′ represents a hydrogen atom, an alkyl group which may have one or more substituents, an aralkyl group which may have one or more substituents or an aromatic group which may have one or more substituents, and
a represents an integer of 1 or more, wherein when a is an integer of 2 or more, R₁, R₂, L₁ and Z₁ in each of a plurality of structures may be the same or different from each other.

2. The electrophotographic photoreceptor according to claim 1, wherein X in formula (1) in which a binding site of X is replaced with a hydrogen atom has a structure selected from the group consisting of the following formulae (A-1) to (A-13), 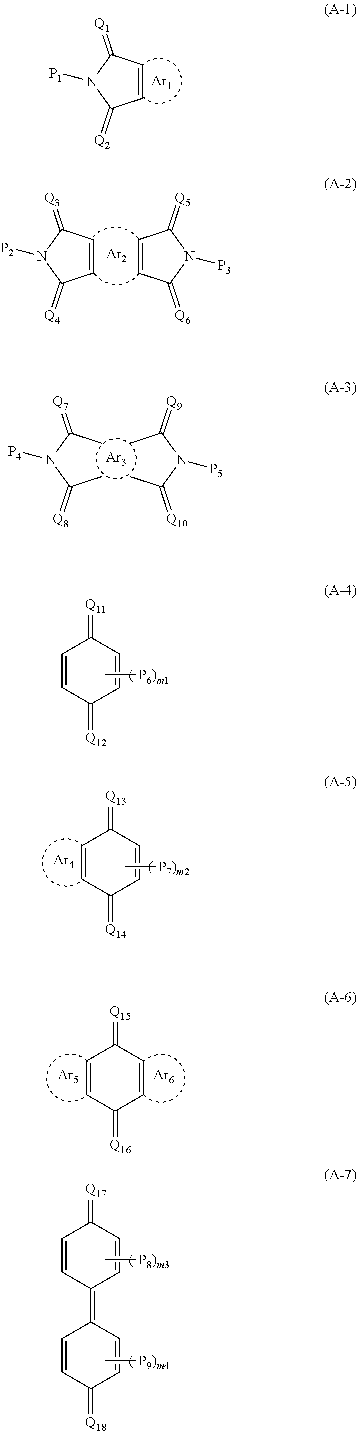 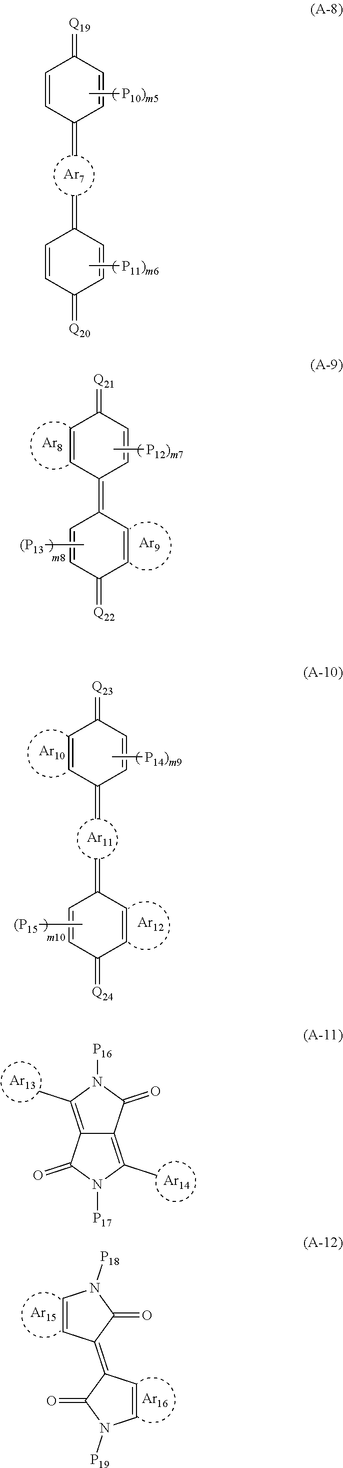 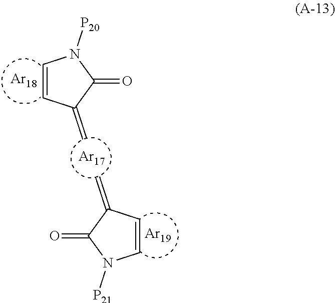
in formulae (A-1) to (A-13),
P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom,
m1 to m10 each independently represent an integer of 0 or more, wherein when m1 to m10 are each an integer of 2 or more, P₆ to P₁₅ in each of repeating structures may be the same or different from each other,
Q₁ to Q₂₄ each independently represent at least one selected from the group consisting of an oxygen atom, a sulfur atom, C(CN)₂, CR″CN, CA₂, C(COOR″)₂, CR″COOR″, NR″ and NCR″, wherein A represents a halogen atom, and R″ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthiol group which may have one or more substituents, a silyl group which may have one or more substituents, a siloxy group which may have one or more substituents, an aromatic hydrocarbon group which may have one or more substituents or an aromatic heterocyclic group which may have one or more substituents, and
Ar₁ to Ar₁₉ each independently represent an aromatic group which may have one or more substituents or a heteroaromatic group which may have one or more substituents.

3. The electrophotographic photoreceptor according to claim 1, wherein X in formula (1) in which a binding site of X is replaced with a hydrogen atom has a structure selected from the group consisting of the following formulae (B-1) to (B-38), 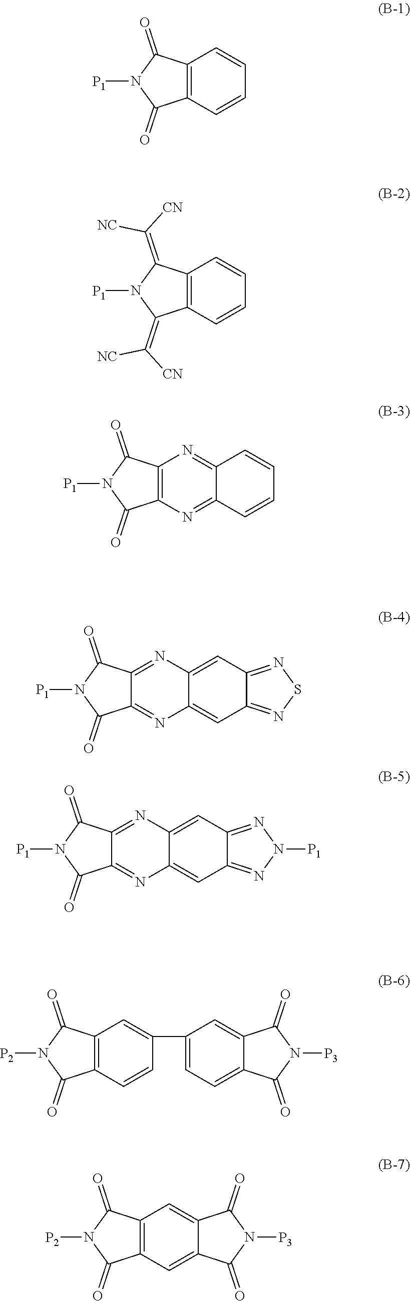 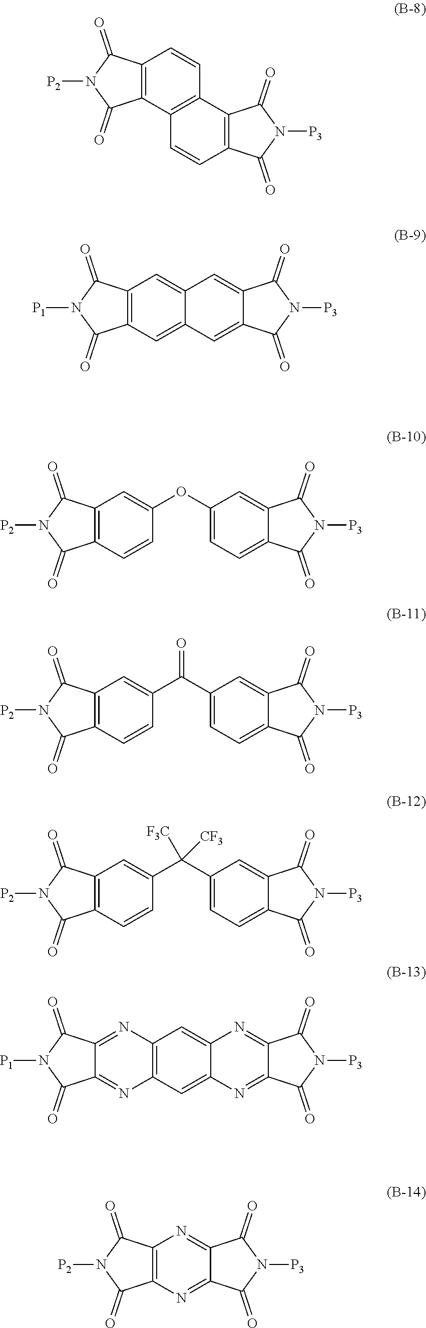 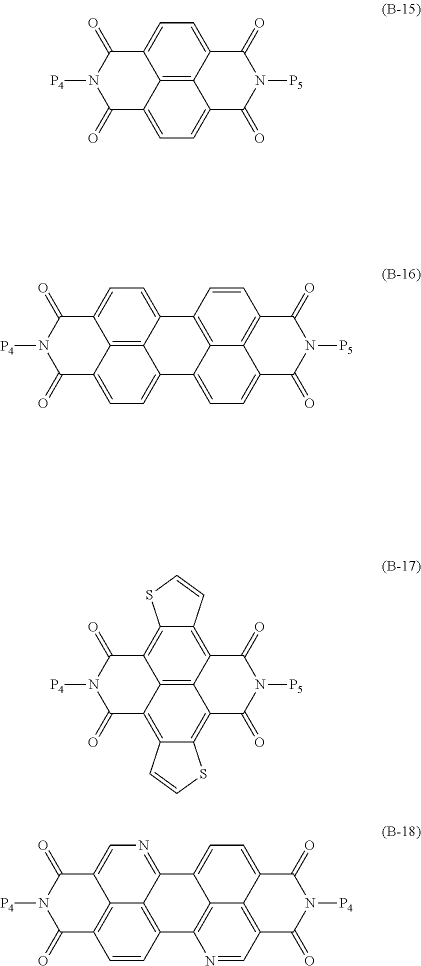 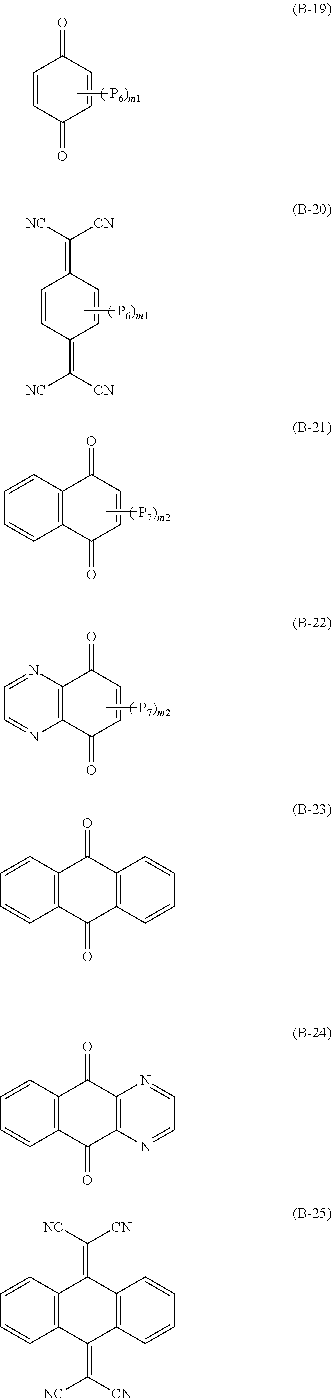 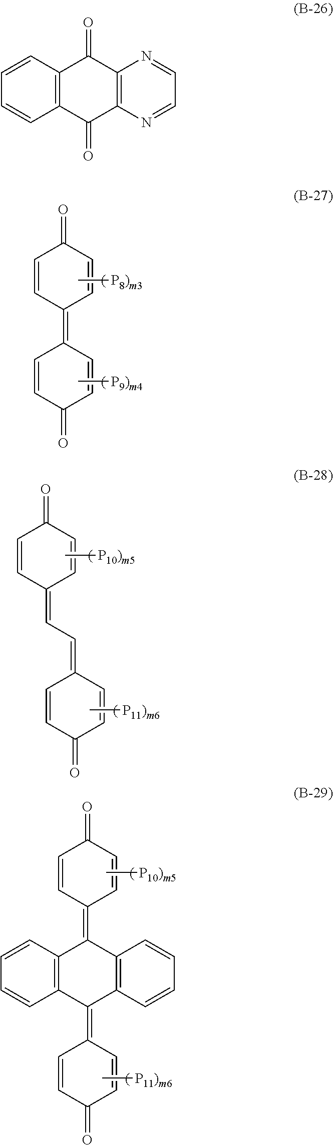 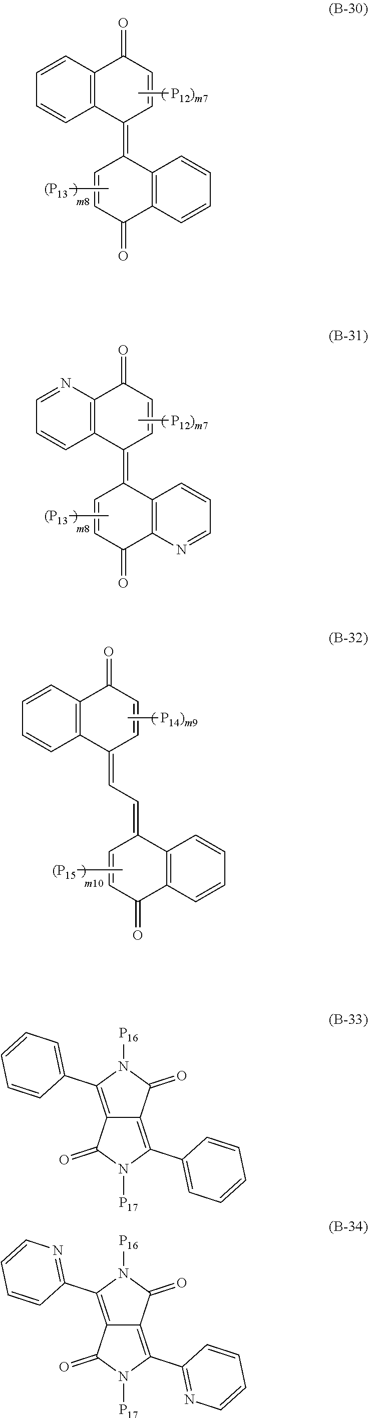 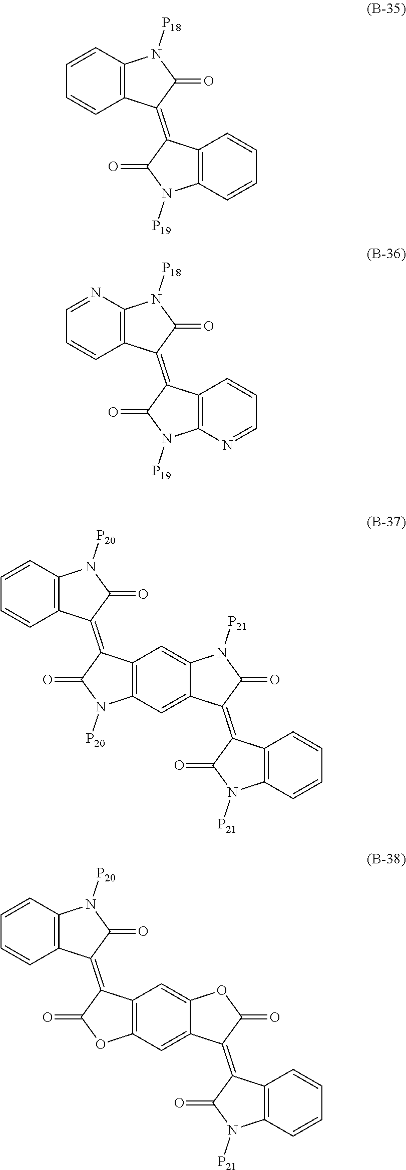
wherein in formulae (B-1) to (B-38),
P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom, and
m1 to m10 each independently represent an integer of 0 or more, wherein when m1 to m10 are each an integer of 2 or more, P₆ to P₁₅ in each of repeating structures may be the same or different from each other.

4. The electrophotographic photoreceptor according to claim 1, wherein at least one of R₁ or R₂ in formula (1) is an alkyl group which has two or more carbon atoms and which may have one or more substituents.

5. The electrophotographic photoreceptor according to claim 1, wherein a solubility of the electron-transporting compound represented by formula (1) in methanol at 25° C. is three parts by mass or more.

6. The electrophotographic photoreceptor according to claim 1, wherein at least one or more of Z₁s in formula (1) are the acrylamide group or the methacrylamide group.

7. An electrophotographic photoreceptor cartridge comprising the electrophotographic photoreceptor according to claim 1.

8. An image formation device comprising the electrophotographic photoreceptor according to claim 1.

9. A coating liquid for forming an electrophotographic photoreceptor protective layer comprising an electron-transporting compound represented by the following formula (1) and a solvent, 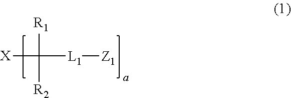
in formula (1),
X represents an electron-transporting skeleton,
R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthiol group which may have one or more substituents, a silyl group which may have one or more substituents, a siloxy group which may have one or more substituents, an aromatic hydrocarbon group which may have one or more substituents or an aromatic heterocyclic group which may have one or more substituents, L₁ represents a divalent group,
Z₁ represents an amide group (—NHCO—R′), an acrylamide group, a methacrylamide group, an acryloyl group or a methacryloyl group, wherein when a is 1, Z₁ represents an amide group, an acrylamide group or a methacrylamide group, when a is an integer of 2 or more, at least one Z₁ represents an amide group, an acrylamide group or a methacrylamide group, and R′ represents a hydrogen atom, an alkyl group which may have one or more substituents, an aralkyl group which may have one or more substituents or an aromatic group which may have one or more substituents, and
A represents an integer of 1 or more, wherein when a is an integer of 2 or more, R₁, R₂, L₁ and Z₁ in each of the repeating structures may be the same or different from each other.

10. The coating liquid for forming an electrophotographic photoreceptor protective layer according to claim 9, wherein the solubility of the electron-transporting compound represented by formula (1) in methanol at 25° C. is three parts by mass or more.

11. The coating liquid for forming an electrophotographic photoreceptor protective layer according to claim 9, further comprising a curable compound, wherein the curable compound content is 10 parts by mass or less based on 100 parts by mass of the solvent.

12. A compound represented by the following formula (1), 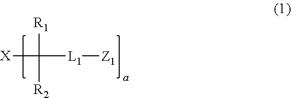
in formula (1),
X represents an electron-transporting skeleton,
R₁ and R₂ each independently represent a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents, wherein at least one of R₁ and R₂ is an alkyl group which has two or more carbon atoms and which may have one or more substituents,
L₁ represents a divalent group,
Z₁ represents an amide group (—NHCO—R′), an acrylamide group, a methacrylamide group, an acryloyl group or a methacryloyl group, wherein when a is 1, Z₁ represents an amide group, an acrylamide group or a methacrylamide group, when a is an integer of 2 or more, at least one Z₁ represents an amide group, an acrylamide group or a methacrylamide group, and R′ represents a hydrogen atom, an alkyl group which may have one or more substituents, an aralkyl group which may have one or more substituents or an aromatic group which may have one or more substituents, and
a represents an integer of 1 or more, wherein when a is an integer of 2 or more, R₁, R₂, L₁ and Z₁ in each of repeating structures may be the same or different from each other.

13. The compound according to claim 12, wherein X in formula (1) in which a binding site of X is replaced with a hydrogen atom has a structure selected from the group consisting of the following formulae (A-1) to (A-13), 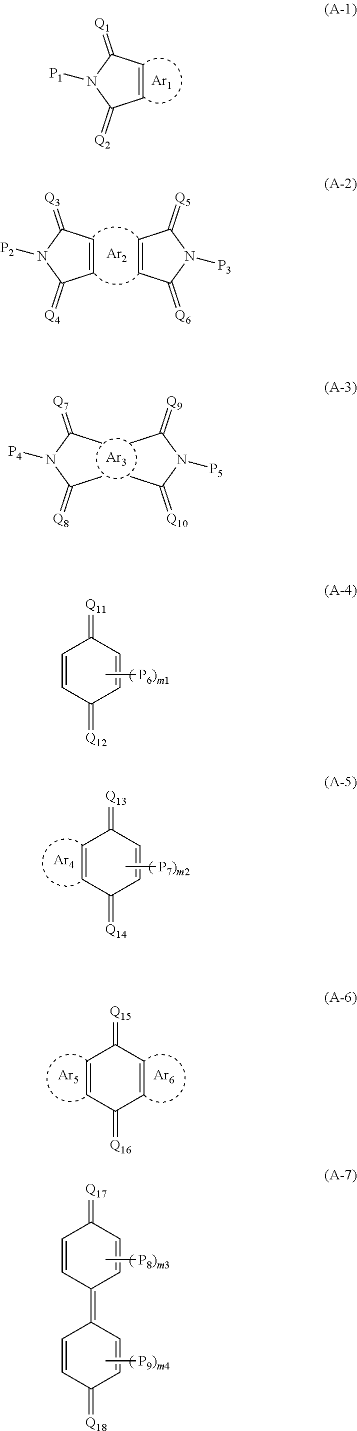 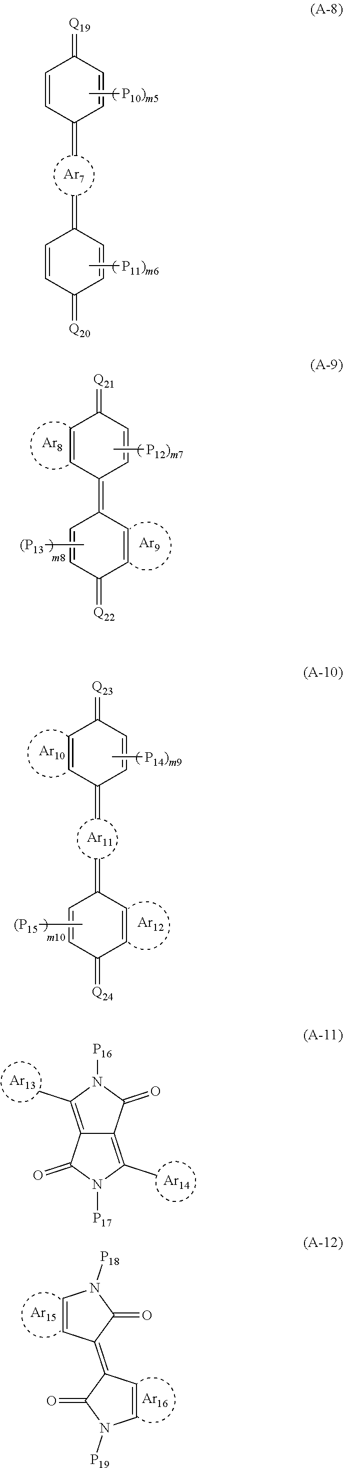 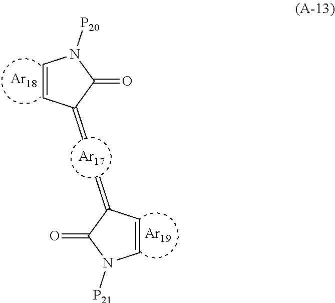
in the formulae (A-1) to (A-13),
P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom, m1 to m10 each independently represent an integer of 0 or more, wherein when m1 to m10 are each an integer of 2 or more, P₆ to P₁₅ in each of repeating structures may be the same or different from each other,
Q₁ to Q₂₄ each independently represent at least one selected from the group consisting of an oxygen atom, a sulfur atom, C(CN)₂, CR″CN, CA₂, C(COOR″)₂, CR″COOR″, NR″ and NCR′″ wherein A represents a halogen atom, and R″ represents a hydrogen atom, an alkyl group which may have one or more substituents, an alkoxy group which may have one or more substituents, an aryloxy group which may have one or more substituents, a heteroaryloxy group which may have one or more substituents, an alkoxycarbonyl group which may have one or more substituents, a dialkylamino group which may have one or more substituents, a diarylamino group which may have one or more substituents, an arylalkylamino group which may have one or more substituents, an acyl group which may have one or more substituents, a haloalkyl group which may have one or more substituents, an alkylthio group which may have one or more substituents, an arylthio group which may have one or more substituents, a silyl group which may have one or more substituents, a siloxy group which may have one or more substituents, an aromatic hydrocarbon group which may have one or more substituents or an aromatic heterocyclic group which may have one or more substituents, and
Ar₁ to Ar₁₉ each independently represent an aromatic group which may have one or more substituents or a heteroaromatic group which may have one or more substituents.

14. The compound according to claim 12, wherein X in formula (1) in which a binding site of X is replaced with a hydrogen atom has a structure selected from the group consisting of the following formulae (B-1) to (B-38), 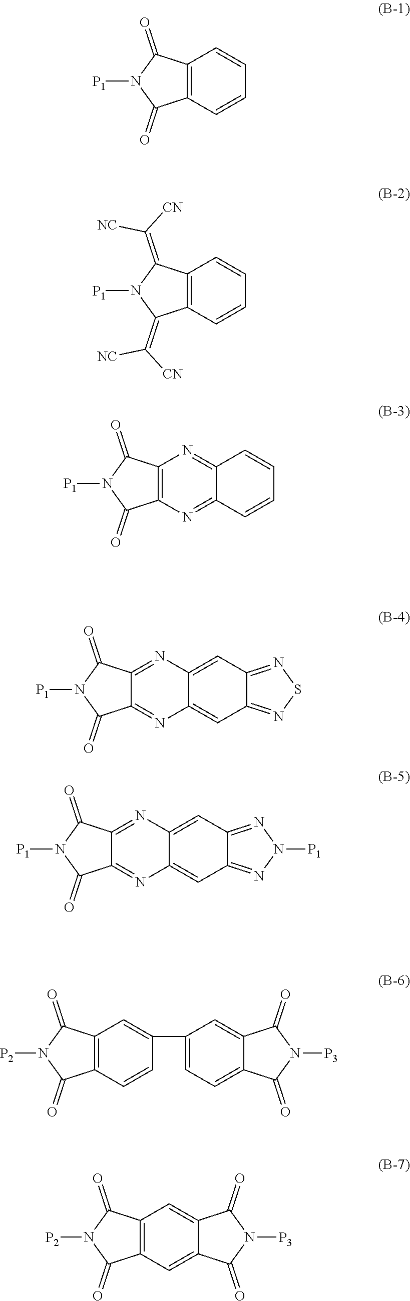 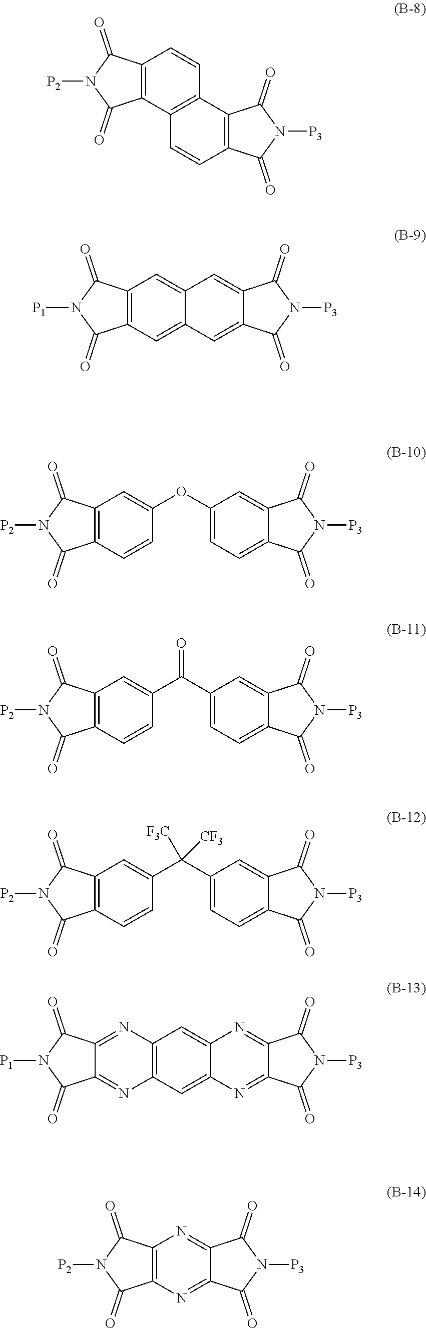 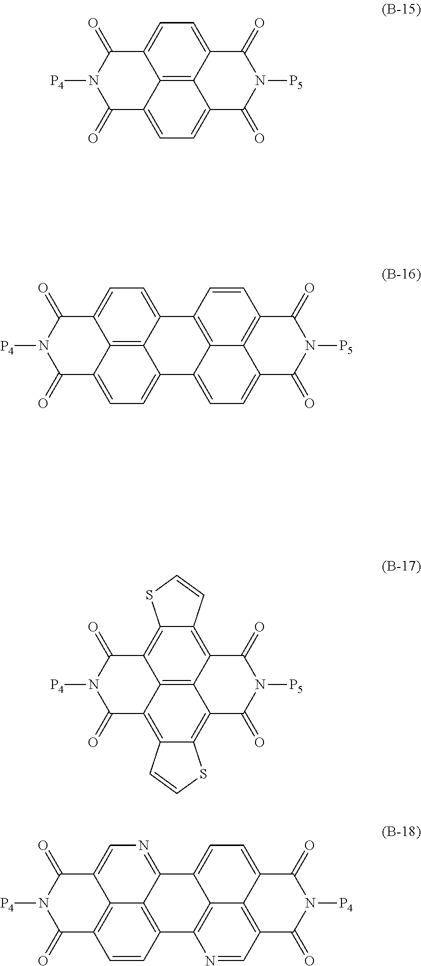 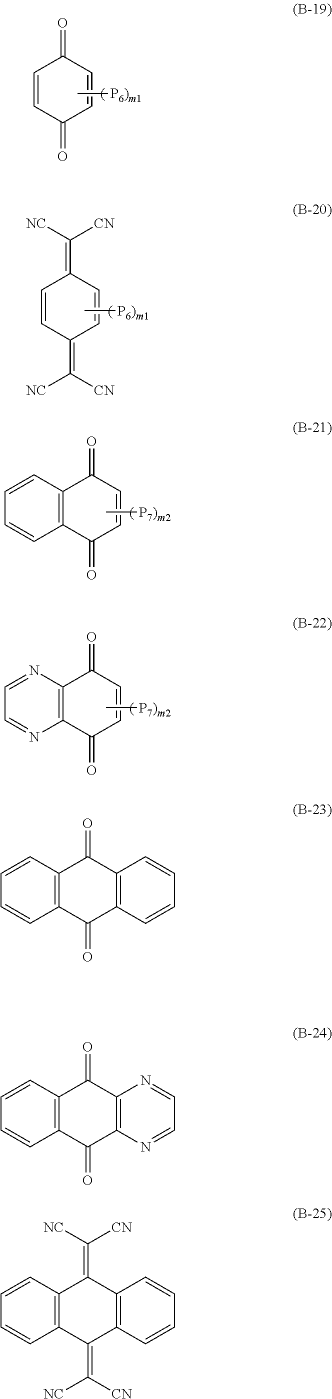 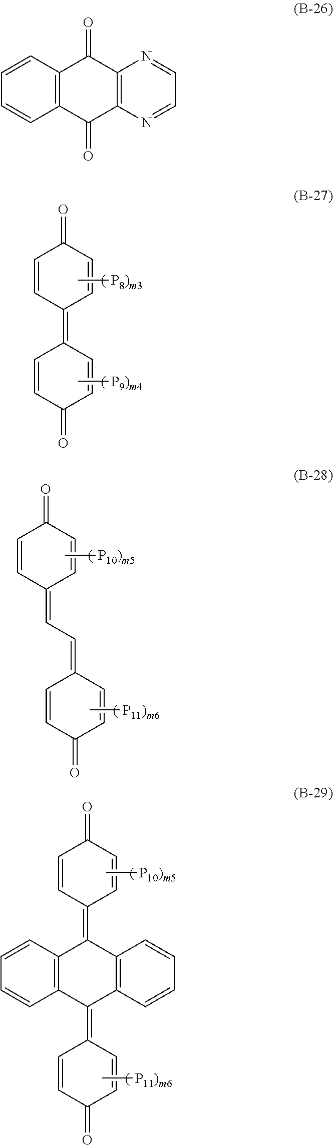 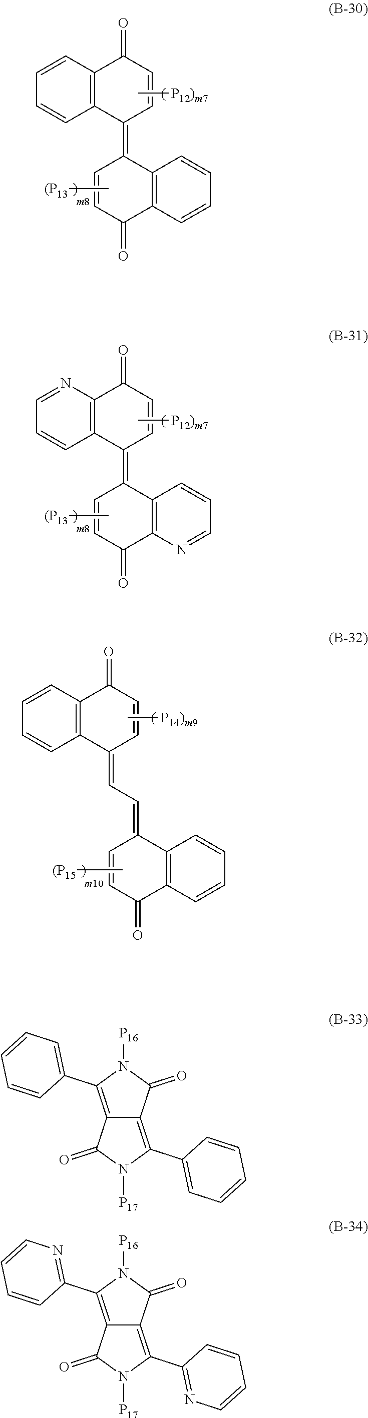 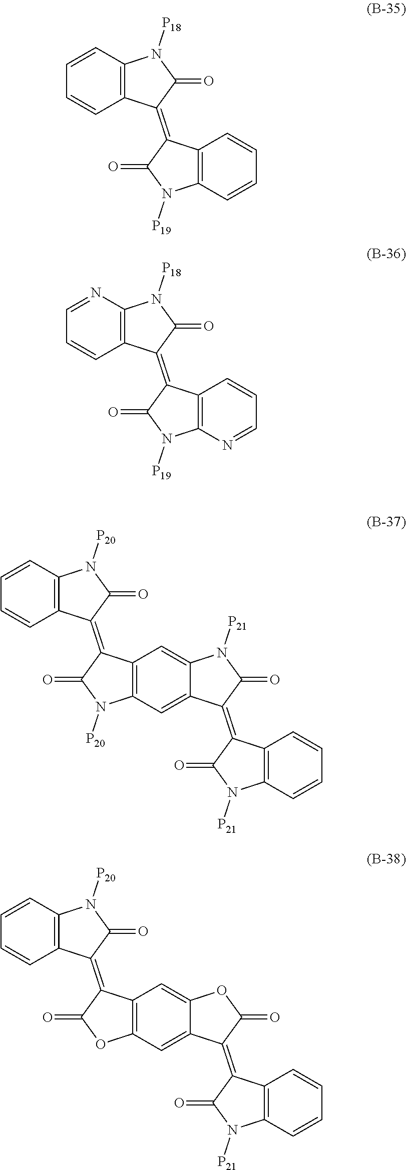
in the formulae (B-1) to (B-38),
P₁ to P₂₁ each independently represent a hydrogen atom, an alkyl group which may have one or more substituents, an aralkyl group which may have one or more substituents, an aromatic group which may have one or more substituents, an alkoxy group which may have one or more substituents, an aryloxy group which may have one or more substituents, an acyl group which may have one or more substituents, an ester group which may have one or more substituents, a cyano group which may have one or more substituents, a nitro group which may have one or more substituents, a sulfone group which may have one or more substituents, a hydroxy group which may have one or more substituents, an aldehyde group which may have one or more substituents or a halogen atom, and
m1 to m10 each independently represent an integer of 0 or more, wherein when m1 to m10 are each an integer of 2 or more, P₆ to P₁₅ in each of repeating structures may be the same or different from each other.

15. The compound according to claim 12, wherein at least one or more of Z₁s in formula (1) are an acrylamide group or a methacrylamide group.
